SEQUENCE LISTING (60 sequences)

SEQ ID NO: 1 - DNA, 369 bp, Homo sapiens
tttcctttttaaaaattaaactaagtggtttattactggaaatgagataattttctttactatgcaaaaagaattttaaaagccctttatttcaaaatgttttccatgctttggcccccaatatcttcaatattaaaataaaaatgatatatttcatcacaaaaagaactgtgaaaaataaaaatgtcttaaaatctgaaataccttttgaaatattactgcctagtcactcaaaaatttatgtaacttacaattctggtttaatttcttaatatattatacatctaccaagacaattttcccaaatacttaaattttaagagggcaatcttcttgacatgcaatagtgtaaaagcatgttttattgta

SEQ ID NO: 2 - DNA, 834 bp, Homo sapiens
agtaccaattatttgcacactttggaaatgtgaatgtttcagcaatgtttcatagcttgaaaaacaccgtataatttggtcatctaacacctagttattgattcaagagaaatgttattctgttggcactgagaaggctgttgaatgaggaaggattagatacatattttatgaaggtcatctttagaacaatacataggctgagttaagatactggatttgaattattgaacattactgggtatcacaaaataaaagtaatgcttctgggggactttactaaaaatacaaattctcatttatatttgtatttgtattgtagttcaaggttggaactcagaagtgtatattttaacattggtggcctttgataacattgtaaaggaaagacaagttaaagtgaaccaacaaataaagagactgtggaaagtatccctatggaatgtaaaaaggatcttaagcgggactaattgtggaaatgtaaggatcgagctttgaaattattgcaaaggcataagtagaataaaatgctgactaatccacaaaataggcgtttacgaggataattcccaggtttggactctgcactgttgaatactactcgaactactcaaatggaaaattctgataggacgcaattactcagcgttaagcgcggccaaccaaaccctggagttcaccctggcctttcctgagatttaagggcgttttcgctgaaacacctacgtagcaactgggctcgggagatggaacgaaaagggccttttcaaatcttgtcgccccaaaagaaacttgaaactactcgcagaacggaggccctcccccatcataaaa

SEQ ID NO: 3 - DNA, 162 bp, Homo sapiens
ggcaggtaccctcattgaaatagaggatagagattataaattatttttttctgtcttctttttacctagataagaatttaccacatttagtctggtaaataaccatctgtatggtcacagagagccacacagaaatgaaaaaaaaaaaaaaaaaaaaaaaaa

SEQ ID NO: 4 - DNA, 501 bp, Homo sapiens
ggcaggtacttgaagtatattctctttttggattgctgtctaattttaatcacaaaatacagaatgtaaactatcaacaccattttgcctactatgcattttgcactttaatagctgctacttcaaaaaacagaactaaaggcccattctttggaaccatttcacattttacaactaagaagcaataccaagaataaacaaattcatataactcatcaatatgtattgactatttatcttaatttttttctttcttaaatatcacgattaaaaaaaacattggcagcatttataagaactaagtagttataagtagttcactaagaatgacctcatcacaggttttagctgcaacaatataaaactaaagaggtcaaatttattctccaggctgcgactactttttcttttctttttccttgaagcatagcttactttgattttaacccttcctcaccacccctcttcctctaagtgtaatcaagcaaggaaaattccact

SEQ ID NO: 5 - DNA, 460 bp, Homo sapiens
caggtaccatctctccagcatgttccagcctttatcagtcagtccaggcttctataacaaaatatcatggactgaattgcttacacaacagacatttctcacaattgtagaagctggaaagtgcaagattaaggtgctagcaaatttgtttcctggtgagggtcctcttccgggcttgctgtatcctcacatagcagatagagagagaaagagagagagagagagcactctatagactctcttcctgttcttataaggacactaattccgttgatgagggccctatgctcatgactttgtctaaacctaattaattctcaaaggcctcacttcccaatactgtcacattgtggggtagggctttaacatatgaaatttgagggaaaaggaatgttcagtctagaacaaagccccatacatttcaaaaagctgtgggacaggagggaatggggatggtatt

SEQ ID NO: 6 - DNA, 282 bp, Homo sapiens
tactttttttttttttttttttttttcctttaataagcatcgtgtttatatataaatggcttacatttttccatgtccatatatgagtcacacatgatgaaatgcttgatgacttactcctttttaaactaggtgcactgtgggacaccttttatctcagtgcctaaattaccattgccatataataacagcactcaaattaagaaccgtttccactaaaattctatttttaagaagcaatattcatttgttgctctactatgcttctttttccatgcagta

SEQ ID NO: 7 - DNA, 740 bp, Homo sapiens
ttttaaatttttgaggttatagagtttccataaaaagtcatcagtgataataaattttatttgactttctacttattccggattctgcacgagctgacatataaacacgttacttatggatcactttgagagaggaaacactattcagaagatttaaatcccagaatttaatccattgctttattgcaagtttacagaagacttgtcttcagttttatctgcagtagttgatctattgactttaaatgttgaaaacttatactaatgttgcttactgtccaaaattattcctgggagataaacattcagattttctacaataaccactgactggccccttagcagccattttaaatgtaaatttgatgatattgcccaaacatccccagtccagttcaactttcagatcctcaagcctaccatagtcctttgtgtttgatatttctttcttatccttaaatgtggtagctccatgagcacagggacctgtgttttgattcactgtctgcatacttcactttgcagaacatttttaaggtccatgcatgttgtagcatgtatcagaactagactccttttatggctaaatattccattgcctgcgtatagcatatttatccattcagttttcaatggacacttggggygtttccaccttttggctcttacgaataatgctgcaataaatattgtcatrtaagtatctgttcaagttcaaaaaaaaaaaaaaaaaaaaaaaa

SEQ ID NO: 8 - DNA, 1811 bp, Homo sapiens
gtgagggagacagtagatggcctgggatgacttgagtccatcatactattgcttggcaggtgtcctcccccatgtttgattcaaattccatgagtgacctacctttccccaggaatgggactgagagggtagtctccagcaactcagtctgcacagggctccccgttcaggctgcctttggtggttgtgcttttgtaagtttctttctctgcacttcgacttacctctgaatcagaaagcaagcccagcaggtgaatgagggatgtctgcttggcattgcccaatctaaccagggaggctggctggccacccactgtccgctagaggggagagctagcaggtgttggtatgaactcaggaatagaaacacgaggcctttttaaatacgagggagaagaatccatgatgcatacctgtaaccccctagaacccaagtgccagaattcctagatgctgcttctgtttgaacaaaatgtcactgcttttacacttgaaaaaaacacactcgaaaaatgttcaactccatgaaaaatattttttggctttaagaaattgtttggtgtttaactgtttcctttgattgccattccaccagtaaattgttggttgatttgcactgcactctggggttggggttgggaggggagggtccttatacagagccgaacctggggttgctcaggaagtgggccagggaatgtggaagtcgttgacattgcctgggccaaaagagtgggagatagtttttctcccctcagcccactcctggtagcacctgtcgccagcctggtacaaagccaggcctttttcttctgtgagcatctcatcactgtccagcagcaggtggaaacaagggggacaacaaccagacctatttttttctcccccattttttccaaattttgctgtgccaaatgtttaaaattttataaatatgaatctattgaaatttccttaatcaagagcttcttcgtgtaaagtttgcttttttagctatagaacaaagaaaacagtaaatatctcttaatggcatccagccttgctgagctcaccttttttcctgaagaatgggtaggagtgaaatatttaatgtaaacatttcaccaagtccctttaccctaattttgaagctgcattaaacccaactcactaacacagggaatgattgcaccctagtcctctgtgggccaagaaactttcagaagcattaaaaaaactagttgaagtatatcacttctcaccaagtgggtagagtcagttggctgtttgtcccttgttttttatttattccataattatgtttgtgctttttgttttgtaaacagtaatggaacgtacatttttattttgtttagaagacaactttgattcaatctttcaagaactgttccattcttggtttcttcttagggggataaaaagttaccagttaatttgttttgagatatttaagcattctttgagttatagaattgtgatgcagggatttgtgaatgagacatttacatgtgaaaggtgacttcactagttacctgcttgagcagagtaaagtgtgtatatgtacataaaatgtaagtaatcttaactccattgtgcagtgccttttagatgttccgctttctataaagtcttcaaatttttgcatatatattatatatatatatgtaatgttatagaaatatatgtataatatacatattttttccaggggtatctgatagctctgtattttgttatggaagttgaaagaaaaaagtattttacctcagaaattaaataaaaaaatacttttaagtaaaaaaaaaaaaaaa

SEQ ID NO: 9 - DNA, 608 bp, Homo sapiens
tacagagtctctctcatcactttcatagcaggaccctacttaccgataattcatagcatacctccccttattttaaaactctatgatagctgatttcctagctgtagcaatcaggattcttagaaagaatcgaaactgaatttagctaactaaggaagcggatttcattaaaaatattggattagtttacagaatcagtagtggagaaccaggattgcataaaaggtaagaaccaagggagactggtcaaaagaaataatttgcccagaaagtcaccaccaacaattctaataatgggcacacgatgctgacaatattgctagattccaaagtctgtggagtttaatcaggcaagagctaatggctggccaagccttagtcattagtcacatgctccctcagtagcatctacgatctttggattcagtagcagaaaatacataccgcgttttaagttccatacctttcttttgtcccccaaagaaagcaggtttatacgctgagtcaccaaaaacaaaacaaaaaacacccagcaatattcaaatattcaataatgtcatcaaaaatttcatgatacccaaaggttttgtgtgtgcattagcaatgta

SEQ ID NO: 10 - DNA, 383 bp, Homo sapiens
ggagctccccgcggtggcggccgcccgggcaggtacgcgggggccctacataaaaaagattattaaggctaggtgtagtggctcaggtctgtaattttagcacttggggagccaaggcgagaagatcacctgagctcaggagttccagacatacttgggcaacatagtgagaccttgtctctacaaaaaaatttaaaaaaaaaaaaatctgggcatggtggcatatgcctgtaagtcctagctactcaggaggctgaggtgggaggatcacttgagctcagggggttgaggctgcagtgagccatggtttgtgccactgcactccagcctgggtgacaatgaggttctgtctcaaaaaaaaaaaaaaaaaaaaaaaagtacct

SEQ ID NO: 11 - DNA, 652 bp, Homo sapiens
gcaggtactctgtttctgagggtgggttttttgttgttcctgttcttttactcttttatatacatacagattggtgaacctaattttctcactttttcagtttatttctttgggacagttttatggccttagaaatatactagtcattatatttttttcattaaattcctttatccaggaattatgaataaatctggcattatcaaaaagtttagtcacttcaactccatgaataggaaacaaatggtaacttgacatcagaaaaaaaaattgaaccctgttcattttctctatctgaattttattatattttatgctacttattttaaaaatattacttattttattgtatttggtgaattttattttattctacttctctgaaaaaatggtcatctattcatagttcgtgttattcctccagttaactaactgatgcagcagttgatgggaacttctctgtaacactagatacatgagatattataatattttttctttttctttagttgttgtatagtcgtctatctcagctagtagaatttaaagtgaaaggcataaaaatctgaggtgcagatatgaaacacagcagttgttttatatataaatatctggtcacttctgtaagcaattttttttttacttactcaga

SEQ ID NO: 12 - DNA, 539 bp, Homo sapiens
gatgaccataatcataacttttggaaactgggggatctttgcccaccttcccaataaatctgggagtatacgccattttataatctaactatgtcttattgtccaaaataacattaccaaaccctgacatttagtttcaaaagtaaattcagcaataaaattggatcccagactttaaggagactatgtatgtcagtgttacatctattgcatctaatataggaccctagctgtgaaggcctttaagaatagtataattttatggaagcatgccttttttggcttgtaaagactaccctcttgactttcagaaactaagtcatcaccttcacgtataattaaatcaaagatgcaaattctcacctaaaatttactcatacatattttagaaacccatagaactttaatttctttttttctatacggcaacccaaatccttatgaatgcgataaggtaataagaaagatgtcttaaagaaaaaacagaataactgttacttatgggggccagacaatcagctgttttttacttaagggta

SEQ ID NO: 13 - DNA, 1524 bp, Homo sapiens
cccacgcgtcctgtaccctttctattacatgtattcgatgttttcataagcctaggaaacatcgattcctttttaataatgtcaatctgattatttaaagaggtaacaattatctgttaatgcttggaaaaacaagtagggttgccttggaggccaggcttcttagttcattcaaaaatattccttggatttatgccatgtattaagcatttttagcccccagtattacaactgtgaaccaaacggataaggccctaaccattttcagcattctctttggatggggtgggattggggacttaattaaaatagagatatagaaaaataggcatctaaataagataataagtgtggggttgaaatgaagcatctaacaatagttgaagttagaagtaatattttacagtattgtaacctctatttaagtttgggtattagttacagatagcataaaaaagccttaatttttcactttccttgctggcaaaggtacatttatttagactgtccatttaaagtaatgtttaacataaacattactgtgaaaaacattccattacatattcccaagcaaatgagctgcatcttctttactgtattttacaatttagtacaacagttttaggcctcaatcttaacatcactggtattttaaatttggcaatgaatatgaaattacttttgacttacagattgattatattattactttgaaaatgcattaatttcttagaaaagtttggagcctctatctttttttgagttaatacttaaattctcattacttatattaatagcctgtactaagtgaaaatattatttatgcaagtaaacaagtcactataggcttttaagacttttctttaattttgagattttgtcatcaaagtttaaattttttacctactgtccacttaaatataatttaacagtttgtaaagtgaaatagttttaagtatgatgtatgatgcacctgcatataaatgaaaatggggtgcacaaagacactttactatgggaactgtactggaagatttatgaaagcatgtgaaattgcacctaaaattgtgttattagtgactataagcagcaatgctaaatttattgtacttgatgaatgaatgtatttagtcacagttactttggtttaaatgtataaatgtctttagggtttttttttaaatgtgtttgtaatttgtactattgtgggggtatacttggactgcaggggttattgtcaatgtgtgatttgtgtttttattttatagaatcatctaatgtgatataccaatttttataagtgatatttacataattctaataactgtatatttgacaacctattaaaatgttttgcattggaacttttttcattaattgtaacatgctaacgtggtttaaaaataactcattcattcaacagatattgaatgttgtgtatcagaaactataggaatcactggggaatcaaagatgacaaagtgcatcagtggaaagatccatgctctcacgacgaattgggga

SEQ ID NO: 14 - DNA, 1886 bp, Homo sapiens
tttaaaatggacctggttgaaaagagatttcattgattctttttaatgatattggaaaatatttatgcaattttattttttaaccttttctccatttcattagttatactatttactttgataatcacaaaactatgtggaagaaatcattctgattatattttatgaaagagaggggtagagtaaattttttaattagacaatgttgatatgcagaaatctctcaaaagaaattgcaaaggaaaaattacgttgagatgaaatgttgagaaataaggagtatcttaaaaacctaatttcttacactttttagagcaatcaacttatttccaaattattatttcatgctgttgtttccatgaattgtgtttttagcatagtatatacttttctaccacgttagcatttacactctaattactgaaaataaaggaagaagttggtgaataaaagccagggaaactgggtttattctcattttccaatagcactaatgtataatccctttaaccttccatatggtgctttgtgtgatgagtatataacattcatatacgttattagtgccttatttttttcacttgactttatttcttatctaaagagccatatattttttgtgagtgttctttggtccctctatttaaagtgctaaggagaaagcatatctttagggatgtatataggctgcgtttgctaccttaatctactgtattatctctaaaacaggttagccaatgtatttttcttaaattcattgttcatttttaagctaacctatacaaatggatattaaacaccagaaattaaagccattaaaattgaattgtactttaaaaccataaaaaatgctaattaagtttagaacaggagttgaaatgactctcagttttatctatagctccaattaggacaaaattactttacaaaaagcattaaaaataaaagtaactgtatggtacttcaggaatttgaccagttccttacaacttattcttggcaatattttgtcagtaatatactttcatacagatgctagagaaccatggtgtctgctatttacatgttatttgttcccagtgagctttatcaacatcaaccctgtttatataatatgttattttgctcttcttgatctgtgccttttgttttagctttatacaaaatgctaggtttgttatattctttatgtatcataatatataattcaacttttcctgatgaatgtatgctgaataattaaactaaaccatgtgcttagaaaagacaaagacaggttgtatatgcagttatgtgtaaatatttgacaaagaaaaaaaaaggaaaagaatcttcctgccaacaggtaggtcctgctggacaggacctgaaaattaagagatttttttttttaagcagtgaaatttaaaaatctattacctcaagaattttcagtcgatatgtcaatgtactgttccttctcagtaagtacagtgtcacatgctgctggcggtaaaagcaacccaagtgtcagatcacacaaattttagggacaagtctttcaaaagacggacaccttgtgatattttggagtctaatttccgacttttcgtaatgtcccaatgaccttttgttgtggttgggcttttataaaaaattattttcggttggttgttgtagaatggaagagtaatcttaattatgaaatcagattgcatggaagaatataaagacactcactcggatgaaaactataacggttttcaaactattttgatggtcattgtacgagctattgtatggattactgtggagtgctgtttaccacatgatgtgtgcaatacatcatgcaatgttatttagcaatgcgtaataaaagtttttaaaagagaaaaaaaaaaaaaaaa

SEQ ID NO: 15 - DNA, 674 bp, Homo sapiens
ccgggcgggcggcagcaggcacgtgcgcagggtacacattgagatgtatatgtacagttacaccgaatgaagaaacaaaataatgggcgcatcgcaacaccggatcctaacattcatgatgtaagtcctctatcccagcatatgaccctcaataaatagatgagtgggaacagcggatcatgaatatgaccaactctctcactcgtgcgtatacaaattgtagtccttcctatgaataaagcagtggagcataggacttcttggacaacttcatacccagaaacttggggagaagccaaaccaggagggacttcatgcaatgggaaggctttgcttctctacaggcttggaattaaccccagggaaccattatctccacctgttgctttgagcacgtaaagtccctgaatgaaatgcttaattttacttaatattaaaattggcagataaattgcaggacactcaaatgtgaatttcatataaataatgaatgaattttgatcacatcccaaatactgcagaggacatatttatattaaaatttgttttcattgttcatctgaaattcaaatttatccgggcatcctgtatttgtatctgttaggtctaaagagacaagccatatacaactgttttttgtaactctcatcccaaaaaaaaaaaaaaaaaaaaaa

SEQ ID NO: 16 - DNA, 1011 bp, Homo sapiens
taaagttcctccatagaaatgtgtggcatgggaacagagcctaatactttctgaataagatattaacaggcttgtagtggatgctgggaatttagaaagctctgttttttttttttttctaaagcctcccagtagaatgtcagactaataaatggactgcatatttccacagtgtttttatgacatgtttcacacactgtcttggtgcagatatgagaaagcccttgtccacagcccaggttctgactcactcattaattttaccttgtattcaagttttcttggggtggatctagactagaatagaatagaataagccgacctttaactacagtccttaaatgaggaaatctccaccctgggccagctgtgccaggaatcccgacgagatggaactccgctgtggtttataggcagggctgattttcaggggggagtgcacccatctggactccctgctgttcccatcaatctattccagccctggtacccctggaccaccctactgtccagcaactaaaggattaacaggagccacctccaggagcctccgggacccttcagaaggtgtctgatttgatggatcagaggctaggccactgccagctctctgcacatattttacatatcacccatttgcaggtgttgaagaccggtaagtgcccaggagctggcaggtgttgcaaacatgtcagtgtacctggtgggggcgctcctgcccagctgataggagacagcttctacccagcccacccctttggtggctacaccagcttggcacagtctctcattgctgaagagcatccccccgccaactccgcccaatccccacctccagtcttgactacttttcattggtttgtttgtttaaattgacatctcctgattttcaaaaccttgctgattaacaaagcaaaaagagcacacacaaaaaaaaaaaaaaaaaaaaaaggacgcggccgcaagcttattccctttagtgagggttaattttagcttggcactggccgt

SEQ ID NO: 17 - DNA, 1279 bp, Homo sapiens
tcttgccttcattaatagatttactaactgtatttatagtaataaaaatcctttatgtctttatatgtacctgtgccaacatacacaaattaatgtaatatcctatattaagggaataaaatataaaaattatatgattatctcaatagatgcagaaaaatacttaacaaagttcaacatgttttcatgatacaaactctcaacaaaatacatatagaagaaaatttcctcaacataactaaagccatttataaaaagccaactgccaacataattttctttgaagttttaaagaccatggatcgatttttctctttatgtagcctaatctgatttttatattcatatctgttaaggcatacacaatagaaattatatgttgacactacctttagagtccaggctccttaacctatacatgcatttgtaactgtatcttacctgttatttgatgctaaaagatggaaaggttttcctctaagatacagtacaaggcaaggtacaaagatgcccaccctctgcacttctattcaacagttctattaaaattgctggaaatactagcaaggacaatcagataagaaaaataaatagaagccatccaaatgcaaaaggaagcaaaattatctttgtttgcagatgatatgatcctctgtgtagaaaatccttaaaatgtcacaaaaattgttagaactaatgatcgaattcagtaaagtagcaggacacaaaatcaacatcaaaaatcagttgcatttatttacatcaataatgatctatctgtaaaagaaatcaagatggtcagacagtttttataagataatctgtggaaaatatgtaaaacttcttctgatattataccattcttgtatttgaatgatatgacctatttgtatattattttttaaatgcattgctagattttgttggttacattttaagtatattgaatctatactcatatataacattgttagatgtattcacagtgaactgcaacttttatcattatgttcttactatttgttatatttttctttttattgaactatcaaagaagttttctacattattttcatttatgtataaaatcatttatatataacatttatacgtaaaatcatttttatataaaaatatacataatttatgaataaatgatcttacattatttctacaaaaaaaaaaaaaaaaaaaattcctgcggccgcaagcttattccctttagtgagggttaattttagcttggcactggccgtcttttacaacacgccatcacaac

SEQ ID NO: 18 - DNA, 2290 bp, Homo sapiens
acaaacttagaaaacttcatgtactctggagttcacaggtgttttccttttgcatgttgccttctcctttcctcctatcacaaagaatgcttctattatcactcccagtcacttaaatgccattctgcttctttagcaggaaaaaatacctgaatgaattctcttacagaaagacagttggactgaatttgctttttacaatcagtgtggaaaatatcatagtcaattgaggcaatcagtagaaatctcactaaaagtttgtaccccctgagtatgggtaacagcttaaacggcaagaatatataaaagtagcaacagtttagtgggagccaaaaagcactaggttaaatatcatactacgtttttaaagacttgataaatgtgtacacttgaacaaccatccagagggatagttacttgagcattacagagtagtggaactcaaaatctaagattcatctgggatacttctaaaagaaatgtcagagtttggaatctcactcttaaaaatgtgtattcccaggctaggcatggtggctcacgcctgtaatcctagcactttgggaggccaaggcgggtgaatcacttgaagtcaggagatcaagaccagcttggtcgacatggtgaaaccccgtctctactaaaagtacaaaaaaattagctgggcatgatggcgtgtgcctataatcccacctactcgggaggctgaggcaggagaatcgcttgaacctgggaggcggaagttgcagtgagccgagatggcgccactgtagtccagtctgggcgacagagtgagattctgcctcaaaataaataaataaataaaaatttgtattccattgatttgggtagacaccaggaatgtgcatttctaacargctttccaggcgatcctatagtaagtcatctgtggactactttaagaaactcttctatagagaatggagttggattaataataggtgattttttacactggactgattcacaagaacctaaacagtagtccatgaagctgctcatctgtggtaactatttggccccgtctcactctgaaagcagcaggagatgttgtttactttgtttctatcccctttgtctggagattaattttggaatgaaagtttttctctctatgccattcctggttcttttccaaagcctcatacaagaggattaggtcacaatgcatgcattaccttttaaaagaatgcgatattgataccgatgcttacttttttttttttactacttgttttattcctt[450 bp omitted]ctcccttggtatagtggaggatttgaggattggaatatagagtagagtgcttgcttaagcctgggagcccatctttatagctatttgatgtaagaaaagagacatggccatttctaaactatataaggtgagtgtgtctattcccagcagatataaaggaaaaaggaaacttttttgattcccaccttcccagcctcacctagccatcttccagcctcaaatatagagatgttagtgcaaggtcctgggctctaggtgatcatttcataagtcctttacagataaagaaaaagtagtgtttgtatgtttgtttttaagtaaccccaaaacaaatttatattgtattcagcaaaattggaattcaggtgtttaattttagaacatgaagtgcctgctgttttaagcattgacttgtataaaaagaattgcatgtctccagtaagcttatgggttttctcatttttaggtatatggcttttaatcatgtaaagtgaaacattagttttcttgcattttattacaggttctttgttgcaataaagatgctgctgaaatcaaaaaaaaaaaaaaaaaa

SEQ ID NO: 19 - DNA, 1055 bp, Homo sapiens
gtttctagaatggggctagttgtatctaggcaaatgggtaactcttaattattgtatgacatttttttctgtatttaatggttaagatttagaggaaggggcaatatgtgaaaaaaacacgttatgagaatgtttcacatttagaccttgagaaattggtatttatttatgccagttacctatgtctctgaggtgccaagaggatgaattaatattttatttcacttgtggagatattcagaaacattaaagtccttggtcctcatgagaaaagaattttcttgagacttgaaagcattactgcagacattttgctaacacattttttgtgagctgtgttaattgcacctacctttatcagtcagaagttttgctgatttaaatcagatcccagctaacccaagcggggctgcactgaacgccacatctctattgcccagatttcccaggggactctggaaattaaccagaagccctcctgctcgaagaccaactcttgaggcgtatatgatttgcatagaggttccaatgtgccaagttcttagaagtttctgggagccatacagatgattactattccaaatgttctcatcttgggttccaccctttttattcaatgagatttgacttttctactttttcactcccatcttcaccacaccaaaattcatatccaacagtcattgtgtatattatgaaagttggtctcaatttagtcaaacctgggtttcaggggatatagctcacataaaaaggaatgggaggtatcattatatggaaatacctgctgtgtgccagagcatcctttaaggattttgttttctgttgatatttttcaaaccctccactttacagaagaggaaagtcaggcccagagaggtcaagaaaatcactttcaggcaaacaaggtcacatggaaaatagggattcctgtgaactcaccctgtgttcttctactccttcatgctcaaaaactctcccggaaatcaaatgaagcccctgctgaaggggggcctggttaaaggggattcacaaaaagccaaaccctatttgtc

SEQ ID NO: 20 - DNA, 398 bp, Homo sapiens
atatttaaaaagtaaaacaaactatttggaaaaaaatgacagtaaaaacaataggaaagcttgtaaacttcgggcttactatcaaaatcactaccttaaagtaatttactacttttaaaataaagaataaaattaaataattcggcatttaaataataaggttagaaagctgcaactattctatatggaaaaggatagaaaagaaaaagaaaaacaatgaatacaattagtaaatccaaaaggaggatgatgataactgttgcaacctggggagtgggaaaaatgtgactctttataacaaaaactctacttttgtgcctgatgaacacttcataaacaaatttaaaatatatagataaatccaatgatcagtcccttaaaagggcaaatgtaaagta

SEQ ID NO: 21 - DNA, 443 bp, Homo sapiens
cgcggtggcggccgaggtactttttttttttttttttttttttttggtgtctgacagaattactagctatgccattaaagcccagattttctttattggaagttttaaactactaattcaatttatttactctatataggtctatttagattttctacttcttttggagtcagttttggtaattagtatctttcaaagaatttgttcatttcatctaaattgccaaattggttgttgtaaacttgttcataatattcctttatctttttagtatttatagtatctgtagtaatacccattctttcattctcagtatttctaatttacatcccttcttgttcattttagctaaagatttactaaactctttaaagaaccagcttttgatttccttgatttctgttctgatctttattactccctttcttctacttactttggta

SEQ ID NO: 22 - DNA, 385 bp, Homo sapiens
ggcggcccccggggcggtaccacgctcccgactgttcgaggtacgctctgctctctcagtagccaaacagataacagccagtacgttgttatccgtattttcgttcgttgttttaacctcagtagcacatttggaccttgggtgttattatatctttgtctttataataaactcatgcacttgcttctaagattgatacttggagcttgcaacttattcactcataaatgtaacttacatatgctttctagattttgcacttcttcccctgcaccttggtagacctacattgaattaattaatttaattagttcaaacatttattgaagaagaactatgcagtaggctcccaggatcaagcgatgacttagtctaccttcaaaag

SEQ ID NO: 23 - DNA, 1014 bp, Homo sapiens
ggtacttttttttttttttttttttttctataaaatcatcatcttcaccaccttatcagggggcaacttttatttaataacacttgaagtgtttttacttggtcaattatggataaataatccggtttgcctttctacgcttacttaaaggaggctgtgaattgttggaaaaagtccaaaactgcaattatttgatccactattaactccttagtctctcttgttgacttccatatcaacatgtaacatatggggtattctaagccctttgcacgcccaattcaggaaatccaactgctgataggactccccctcctccctatcccatttccccctactcttagcaaaactgcctctttcccaaactcatctcctatccacctcaaaaatgttgtcctttattttcctttcctcctatctcaaaaaagtgtttctccgaattttcaagattagttcctatatctgtgctactgatagcccctcttgctttatcagttattctgtttttcctcttcagtctctcattctacccttcttctttcacaagtgctcaaaagtaaacagatgaacagacaaatcggtcgctaagttttacttacttgcctgccacaaactctcatgcacttattcttctgcatttcagactctaccaatttaacttaggctctcattatccctccctcaaaaagtaaccaaacatttctgtatagccaatctaatcctccaacaaacatcttaatcataatactcatgtattcaaaattcatccacaagcccatcatagaattcaaacacttttttttctggctttcaaatttttcaacaatccggctctgatcaacctatctcataaaatgatactgcattgctatctattatcttttatctacctacaaaattcaataaaatataatctacaaatttcagtaaaatacaatccaaaaaatccctcccttttccctactaccttaatttctccgtcctttattgaaaaaagtgccctttaaactgaaa

SEQ ID NO: 24 - DNA, 207 bp, Homo sapiens
acatagaagggaaaaatggcttgcttatttcaaatagttttgaggatgaagcactatgatggagatccactgaggtaaaaaggatcctcagcttttttgattcagaaatgaaatattgatgatttcaatcaagattgatcaaaacctgatgaaaatatcctgataatcaatgggaagtagaattacagcttattgtgagagtcaggg

SEQ ID NO: 25 - DNA, 248 bp, Homo sapiens
tttttttttttttttttttaaagttggagctactctgcccatgaggtagccacccttttattccttttcttaaaacaagttggaaaaccattgctatagataaacattcgatttcattcttttagaaataatacatacctatatattaatatattcaatatatgtatcagtcatatatatattcattaaaatgttaacacccaagtatttagattaaatgctgttatttggcttataaattaacagta

SEQ ID NO: 26 - DNA, 1132 bp, Homo sapiens
cgcccgggcaggtacagatatgtaaatattccaaaatccagaaaaaaaatccaaaatctgatatatttctggtcccgggcatttcagataagggatacccaacatgtgtttgtaactttcaatactaatgaaattagtaaattttgttttttacattaggtgcctaaaactcttgattttacaccaaaaagtaatagaacaaaaataaaaagctattatatggaatggcatcagagtcactctggagcaacaggaagctaactctgtatatcaaccataatagccttattactcccagaaggacatagttagaagcatttctggttactcttcatattaaaatctttggtgttttggcttcaatacactcccttaatgggtgttattattccattgtaatgaaataatattccatggtagcagaaggaatgcttaaattctgccttactgttaaattctatatagatacttgggttggtcaaattaatttggtgcttcacccaaagccccaatgtatttgaactttaatctttcataggaatctagaaaagcacacaatttaattaccactactattcattatcaaagattgcatgaatgaggtttaaaacaatagaaagaaataaaactttagccccctattgctagatgttctggcccatgagagaggcagggcaacttaagagctttggaggcagacagggcaagactgtaaatcttggagcaacaatttattggctatgtagccttggaaaggtatcattatttttctctacctcagttcactaggtggaaaaatggaataacagcatctaactcaacaaatgttgattattacaaaaactaacgattcaaaaagctaatataaataaagcatcatgttatgcatctttctcataaaaataaatcttaatgaaaagcatcaacttttagctttatccataaaaaataccagacaaaacaaaaaaaaaattctaaaaatcaaaaaaacttcctaatcctgaaaaagtaaaattaattatgttcttttgttttcaaaaaaaaaaattattaattttggaaaaaacattttaaaaaccaaaattttcctttttaactttttcccaaaaattttttttttttcccccaaaac

SEQ ID NO: 27 - DNA, 646 bp, Homo sapiens
tcacttaaatataataatgatgttattaataaacgttagcttgagatcaaagtgtcaacaaggcattcttttctcttttggtggtggtagatttcacccacatattttatgcatctataacaagtcccacatagctggctgattactgagctttgagcaggtagcactggtcactatgcacagattagtatgattactgagctttgagcagcttgaacagatcactatgatataggctctggaaaggttgtgcagatatgcctgatgccaaggcactggcaaaattgttggtaggctggcaagttagttccttctcaaggaaaaaaaagtgtgaatgatcctataggttactagacagacatgcagtttccctttggagaaggttgactttggactttgagaaatacattttttttcttttaatctaaagagatttgaaaataatagatgtccaatttttgtttacctattctgaactataaggcacattgctttgttcttctgtgcctcagtttaccccttttagtaaaggaggtctattttaatcagtgagagcttcaggtagctggaggaatgcttcaggaaacacaaaacacaatgaaaaaattgttgaatcttaagctgcatgttgtggaaaagaactgta

SEQ ID NO: 28 - DNA, 417 bp, Homo sapiens
aggtacttttttttttttttttttttctttttttttttttttttttttgtaactataatatttatcattttaattgatgtgtatagttttacaaaatcaaatagtaatagaaacaaaatagcatcccttgtgcctcccctccctattgctccctggtcttatacagtcataaaaagtcacaagaaaccacgtacaaattgttaagccatttcttctgccatttaccttccaattcctaaggagtatgcatgtgctgttatttattttaaacattatctttttttctccctgtcttgacagatgttatctaactcaagtgcactttgctctccctcccccattcccctggtgtaagcataccacacagtctttgagttcacattattatttgtgtcatatagatactgttcacatgag

SEQ ID NO: 29 - DNA, 628 bp, Homo sapiens
cacttgaagtcaaaatgcaatcacagttcttttgatctttttaatcagtttctccattggacatttttcttgcattgcctcagtaacagaatagaggagatatattttgatgttacttttctcttgtcacttttgaagggtggtgttgaagagatgccaccatcagttggatatataaaagaaatagccaaataaagagataatctgtctctcccatcagcccctgaacaattgatagtggaaaataatgtaatacaatttataatgttcaagataaaagcttattgataattttttatcaataaccatcaaggatgatgtaattacaaaactcaagatgatttgacagaatttttcaaattatttaaatctaaatattcagactgaataaacttcaaattagttaagtggattcatttgatagatgctttattttactcattagccaataaaactcttaagagttttcttaaggtcttaggacagacagatttatgcttcagtgacaaaaatcaagaatttaatcagttacacaaggagaaagtatgtgtgtatgtgtgtgtatacacataaccagatattctcctaagtttttcaaaataataaaaacagatattttgggattcat

SEQ ID NO: 30 - DNA, 291 bp, Homo sapiens
gtactttttgtaacatacctgtgatgctgctccaacatccctgctaagagctttgctgattttaacaagtatgtgattccactgctgtaagccaagggtaaacttgattccaccaaatttgcttaagtgttacttctctgtattaaagcttcaagttcactcactgtctaaccaatgatacacagctacctgaagacattaaaatctttcataaatttattctcaatttttttcttcttataaattactagcatatcaggataaagaataaagtgtgtgaagaggcaaaat

SEQ ID NO: 31 - DNA, 1154 bp, Homo sapiens
tttggagggtaacaaaatttcagggacatataaaagaagttgcttatttattagacaaacaaaagtcacccagcaaatatgccatggctattaaaaatagattgtccagggaggaagaataaaccactaattaaacacttttgtttctcttttttttaagccattaatactgatacctagagagaaaaataaaaaccagaaatactttcaaaagccaagcatgatagactagcatttatcagttacctttttattttcaatcctcaagaaatgcaatcgcccaaataaattttaagtataccaaaacactaagaaattacaaaactcaatagcaaaataactcacgtgttacaaccataattacttctataactagaaaattattatcctcataggaaatcctggcaaattgctgaaaacatttctcttctgagttaatctaggagggaaaaaaaataaaacttcagagtccagtcactttgaagtccttatgcccaaaaagacattatctccatcaattgtctacatgcgaataatttcaaatgtctttgtctgtgcaacagccacatttccttctcttatgatgtttttcttctctccctatcccattttcattttcacagctgtttctggagaataaaaatgaaggaacactttcttgctctgcctctccatgctccactttttacaagtctttgtctgatttactacagagaactataaccaaacccaaaaaaagaatggcaagcgaaagtaggagagaagagtgtgcttttgcgctcagaagccaccacttctcaggtggaaaataagaatgtcatgccatgatgacattctgccttgtgtcacaatgattaatagtcataattttttcccattaatgggagtaaatgcaaccaagtcgatggaatctgaccaattttgcacagcatgggtcagtggagtgctattattttggatggagaaatgagaaccaaaaaggaattatttttccatttagcacctaaagtggaaaaattaattttttcatcccccttttttttccaacctaaaaaaaaaaaaaagaaaatttttcaaaaaaaaaaaaattttttttccaaaattttacccccttttaaaccaagtgttttcttttttaaattgggaaccccggc

SEQ ID NO: 32 - DNA, 875 bp, Homo sapiens
tactgtcttagtctgtttgtgctgctatcacaaaataccagaaactgggtaatttataaataacataaatttatttctcatggttctggaggctggaaagtccaaaataaagatgctggcaggttgagtctggtgagggcccagactttgcttccaaaatggcattgtgttgctacatccttcagcagggactaatgctatgtcctcacatggcagaagggccagaaagggagaagggcctagccttagttcccttgaatccttttataggggcactaatccattcacaagggttccaccctcatgacctaatcacctcccaaaggccctacatcttaatatcatcacattggcaaataacttttaatatatgaattttgtggggatgcattcagaccatagaagatgctctttgtttcccttgaacattactgttttttaaggatttggcctcaaattccttctcttgatagatctccttttggaattacctattctgaaaatgctcatgtttcactaattttcctatgacttttacatctgtatctctcatcttgaattccagttctacattttcaaatgactcatacgtggacaatgcactgtcagcagaaactcaacatttgtaaggcaggactcttcaatttttcctcattttgatctcctccataccctttaattgtcaaggacatactttttaggtgtccagattcaaaattttagaagcatcctaaataccctcctttccctaatccccacttatcccctcagtgacttaatcttggaaatcattatgatcattttcataaatgtccttttatttttaaaatgattttgaagattttttttccataaaaaccttttttttttctggc

SEQ ID NO: 33 - DNA, 1270 bp, Homo sapiens
cggtggcattgcactcgttcgttgttagatatggaaaaccaattgactgttgtatattaaacttgtatcgtgcaaccttgctataaattagttccaggatttttttttttttttttggttggttcctcgtggatttagctatgtagtcacatgtatcgtcacttgtgaaagaaaaacacaatgttattacttccatcgctcatatatccaatcagtaaacttaccatagtgaaaagaatatagctaccacagctagtctctcacatgacagataagatgaatggatagcgcatcaatggttgacaacttctctaaagtaaatacgcgctggctgtctcttttcaagattaacacaagatatgtgtcaaacctacaaacaagaggacgcttattccctcgcctctacagttattgaatacctggagctgcacgacttctatatcaaacttacagaccccgtctacctctagggaggagcaccggactcgctcaagacatcgtatgaaagagtctctataacccgctgctctattcgtcgtcccagagtgaccgtcccagccctagctacatctgtggaaatacccgagttaataccccttagcaggttatcccccgttcgatcaacaagttgtgatagcccaaaaaagcgcccgacacaaaatcaaccttagcctaactattagaaacaaacgaccaaccgcccaggtgcacggtaaccatgaaccccaccgctatcaccctcccgctttgacgcggtgcacaaccgccccccgcgctccaccaactaccccttattctgggaaccacctctcgccccgctcctctttccattacccccattacaattgtcccggttccctccacgccctacttatccacctaccaaaagcccctaaacttccgaaacgcctctcttcccaccagttccacaaatatatttcaatttatcaccgggacaaccacccccccacaaaaatctttattcaccggcctctgggatttacctgatattgcgcttcaaccctctcaccaccagacacatttttatcatcctatcgcgcaggattgctatgccccccctacctacttgaaagaaatcgaatctaaggtttgtctacaaccttacccaccactccccactccaagcagccacatatcttgtccacatttattttttctaatcctcagtaatggaaaccgcttccgctcatatcacccaacctacactgtctcggtttgagcgctgtattggttgttgcttagacaatac

SEQ ID NO: 34 - DNA, 501 bp, Homo sapiens
ggggagctccccgcggtggcggccgcccgtggcatgtacactgtaaagtgacaaaacatgactgaaagaaatttttaagaagctaataaaaaagggcggacagcccatcttcatggattgggaagacttaatactgttaagatggcaatacttctcaccttagtctatagattcaatgcaacccacatcccactttcctttactgcagaaattgacaagctgatccaaaaaattgtaaggaaatggcaagggatccataatagacaaaacaattttgaaaaagaataaaatcaaagggctttgtaacactaaggttataaaactcttataagaaaataaaggaggaaatctttgtgaccttatctcaggcaaatagtttctttgatatgacatcaaaagcatacgtaataaaagaaaaaaatagagatattgaacttcatcaaaattaaaaacttttgcatttcaactatcaaaaaaaaaaaaaaaaaaaaaaaagtacct

SEQ ID NO: 35 - DNA, 373 bp, Homo sapiens
ccatagatagaaatattaatacccatgaaagagaggacaatgaaaggtttgtatcatttgtatgtcacaagtcaacttttttcaatcactcattattagtttaactgtaaaaaattatttacatttagcgtgaaactttcctgtattctcaacatatttccttcgcgtagaaaagcaaacctccagttctctgttctttgcttggatacttgccagtttgtaactcagctatcaaacagtaaagctcacaaaacacttattaaaatgactaaaatccaaaacaccaagagcacagcatgctggtgagatgtggagcaacaagaactttcattcattcactaatgctggcaatacaaaatggtacctgcccggg

SEQ ID NO: 36 - DNA, 563 bp, Homo sapiens
gggaaagaaagggggagcgccggccggggggcggccgcccgggggcaagtactctagaagtgagcattagcgtgtgtgttattgttcgtcgaattcacatcgcaagttacaacatgtgctctatcaacacgatttaaagaaatatggggttggaaattaaaaatgtgctatttcaaaaaacaaatctcaacgcccattccttgggaccatttctcttgttagaacgaataagcacgcaccagaaagaaacgaattcataaaactgatcaatatgtattgactaagtatcttaatttttttatttcgtaatatcacgattaaaaaaaacattgagagcatttataagaactaagatgttataagtaattcacccggaatgaccgtcctcacaggtcgcagctgcaacactctataactaaggaggacaaatttattcgccaggctgcaactactcgtgcgtttctttatccttgaagcatagctaactttgattataacccttcctcacaacgcctcttcctgtaagtgtacacaaacaaggaaaattccactttgaagtatct

SEQ ID NO: 37 - DNA, 1280 bp, Homo sapiens
gcgcgcggcggggaggcacagcctgtttaactcgttaatgctgcatcagtgatagatatttcgcgaagcgggaatacacaatgtgtccagggtggtgctcttttgtgttgtttgtatggttcagaggggagggtggctatatccttgcgagctaaggagatgctcaggcttacacactacctggtgtccagcgaatgactcatcttacagcatcacgaatatgttggcgtacaccaataccttatccacccgttctgactgccttaaatgggtattacaggagaagactttgatccatcgcatcctgaacgtcatcattggtgagaggacaaccgtccttgtactatgaccatcttctaaacagacatgcatcggaccagaggaagatcggctgacatcgtgtatctgcgtgcctatgcgtttccgctgtagctccttagccctgtggacacagtatttggactgcctgttaagttacgtaggcactgcttgacgggttctcccacacgaagatcctcacgttgacacagatttcctgttcatcttatgtgtctggtcaacttgttgccccggcccaacatgacctatcccttctacgggttcacaatagtaccgttccctaacagaattcctcacgaactgttaccagtctacaggaaaagccattaccctgactctctgactttgccacactcaagatcccctgctctacgacaagggaagcagacgtcagcacctatagtttacacgtttgattctttcttgttactttgacggtcatacagtgttatgcggaaagtatcacaaactaaccgaacgtgccccagcagacatcctccgcaaatcgaaaccgctccccattcgagttgacatgtacaccaacctctcttccctgtctatgcctatattatgtcagcagaattctttaaaaaaattagtcagtttgcctccgctttcggttggactctcgcacccaaagcgtaccgaacccttaaccctccagatgcccccgcgtgtctccacttgtctccaatcctgagggctccgccccctaccctttcctctatcgcaaacccccttatcctcattgacggcgcttttaatccactatgtggccccccccgtccctcgcttgaaatcaccggctttcatccccctatcccattcccccacacccttcatggtgctgggtccccccgacccccttttctccactcataggccagacacctcatctatctacgaataaccccgccgccccctcctcatttttataataa

SEQ ID NO: 38 - DNA, 953 bp, Homo sapiens
tggtagtaatgtagtatttaagataaaggggtttagatagtaaagatagaaaaagaataaaaaaaatattgattggataattaaaaagagattttggtgatggggatgaagatctatatagagtgtgattggtagggttgtggagtatgagttgatgattatgggaatgataaggatgtagttggtggagtattattgaatttggtattaaaagtaatggataaaggtgggtgaaggaattttattgtacaaaaatggttggggcgtctcacgggggtgagtcaacttaccctattagacgtttatactctctatcacatctgggcgtcatgacaacaaatagccgccgctattcccaagtccctcaactgggtggcgtgatacgcgcccgggaccccccctttaacagtcgtttgcccgcgggagtgttgtatgcttcccccttgacgccagtgcaaaaacctagatcctaggcccctaacgcaattattatgacactatctcacaccatgggcatgcgggcattcacgtcgataccattaaccttgtttatttcccctgtgttgcgaccaatattgttttttaggccagagcctttactcaaggggtttagccatttccgcgcccgtagcatacgccatcccctctcctaatagtagcattaactgcaacgaagacatcctacacgtccctgttatacatcattccacacaaattttcgtcccccaactacctatgatttcccctaacattacctcaaactatcgtctctacaactgaggagtaataccacccgtacaacgtcacaagaatggctaatttctaaaacatgcgatagcctgcgatagactagaatacacaattcatctacaaaaaaaatctgacccaatgaaattaataaacacaataatgacaataccacattgccctacacaccagctacaaaaccattc

SEQ ID NO: 39 - DNA, 660 bp, Homo sapiens
cggccgaggtacattcacttaaatataataatgatgttattaataaacgttagcttgagatcaaagtgtcaacaaggcattcttttctcttttggtggaggtagatttcacccacatattttatgcatctataacaagtcccacatagctggctgattactgagctttgagcaggtagcactggtcactatgcacagattagtatgattactgagctttgagcagcttgaacagatcactatgatataggctctggaaaggttgtgcagatatgcctgatgccaaggcactggcaaaattgttggtaggctggcaagttagttccttctcaaggaaaaaaaagtgtgaatgatcctataggttactagacagacatgcagtttccctttggagaaggttgactttggactttgagaaatacattttttttcttttaatctaaagagatttgaaaataatagatgtccaatttttgtttacctattctgaactataaggcacattgctttgttcttctgtgcctcagtttaccccttttagtaaaggaggtctattttaatcagtgagagcttcaggtagctggaggaatgcttcaggaaacacaaaacacaatgaaaaaattgttgaatcttaagctgcatgttgtggaaaagaactgta

SEQ ID NO: 40 - DNA, 652 bp, Homo sapiens
tgatgtctgagtaagtaaaaaaaaaaattgcttacagaagtgaccagatatttatatataaaacaactgctgtgtttcatatctgcacctcagatttttatacctttcactttaaattctactagctgagatagacgactatacaacaactaaagaaaaagaaaaaatattataatatctcatgtatctagtgttacagagaagttcccatcaactgctgcatcagttagttaactggaggaataacacgaactatgaatagatgaccattttttcagagaagtagaataaaataaaattcaccaaatacaataaaataagtaatatttttaaaataagtagcataaaatataataaaattcagatagagaaaatgaacagggttcaattttttttctgatgtcaagttaccatttgtttcctattcatggagttgaagtgactaaactttttgataatgccagatttattcataattcctggataaaggaatttaatgaaaaaaatataatgactagtatatttctaaggccataaaactgtcccaaagaaataaactgaaaaagtgagaaaattaggttcaccaatctgtatgtatataaaagagtaaaagaacaggaacaacaaaaaacccaccctcagaaacagagta

SEQ ID NO: 41 - DNA, 878 bp, Homo sapiens
ggaatgcattggtatgtgttctcggctcagaaagctatatgagatccgcggtcagggaggatagcaccaccatgggatctacaagaactccgccttctctcatgtgggtatctaactcatcaactactctgtcttgagctcaaatgggacaggcatactcttcagcatagccatcgtacaattgctcttctcgagctactatcacccacattaaggtctttcatgtcctgatttagacgcgagctctgtgcctagcgaggttattcctacccctgttgtacgtgcccgggcggacggtcatgatcatgggcatgagacgggctgatggaaggctggatctagcaaaatcctggcgtcaaatgctaggttttccactaaactatcttaagacaatagagtgtacatctatacgaggagaaagttggctattatactgtccttgagatcacatataacagacctatctcaaggctgtgaaaaaagtcctttaacacgatcaaattatctgtgtagcagacatatatatcaagcagtatagaaactacttacacatgcaatttacagatatctctagggaaaaaacaagcactcacgaatagcagtataacttgaaagagttgagagaacttgcagacgatccacagactagctctatagctgattatacattaggtagagactcttgatatgtctgttaactagatgattttccaaatatatttgtttttccatgtttccctttggatccagatatagtcattacttccttgaagctcacaaaaccctctgaggatatgaatgactgaaaggaaacatccctcatatataaaacatatttcgttacacatgttaaactcccttcttaaaaaatggtggtt

SEQ ID NO: 42 - DNA, 572 bp, Homo sapiens
aggcatttcttattaataatgctagcatttatcctttacagatcttcactttatccacaagacaatatgtaaattaacaataaattgattgtttcattgatcattggtttctttggcaaacatttgtcctatgggaagctgaattaaggtaggaaaatactttttgctacattgctatttggggaaacgctctgatagaaggtctccatagttggtggaattccagaatccaattagcacccgaatgaaatgtaaataataggtctcacaattttatgtcagatgtgctgtgtatgagcccagacatattttcacaaattagttgtcctaatgctggttttattttggtgtattataattcacaaaacgctactgataccaaacaagataatcaatgtgattcctggtatgtactaggaccacagtgatgtgtattgaataaatgaatacatgaatgaataaacctcttgatcactgccccctagctgttgaatttcatgatattcacaggtgttctttactgccttcatttctctcctcctttaaccaaaaaaaaaaaaataaaaaaaaaa

SEQ ID NO: 43 - DNA, 330 bp, Homo sapiens
gctgatcacaccatgctgttttcgtgttttatggagaagtgttcaggtgtggctgctgtgtttgatattaaatctgagatgaacttcaggagatttcctgctttttattacaaataattttggctaggggcagtgttatcctgttcataaatcaggtgttctcagagatttaataatttagaattcacatgaggatttggtggaataaacatttcctttctctgttaagctgcctcttaactttctatcttcccctttctcttcactgcttccatttttttaagccttctttaagtctttgccaaaaaaaaaaaaagaaaaaaaaaggta

SEQ ID NO: 44 - DNA, 478 bp, Homo sapiens
cgggtttatgcctttttgctttttaaaattcttttttatatcatgttcaaaaaggtttcaggagggaataagataagctcatgtgttcaacatactgtgtttaactgaaaacctctttatagtcttctaagattatagctttaaaatgactaatttagggtattagatatgccaagttctatgaagagttcccttgtaattaaactccacaaaaagtaaattcaattgtatttaaaatgccttccatttatcccttttagtaaactaattctgacattcccagaagttgtctaaaaatttgcttctcagtctatgtaagcggtaaacatatttttctggaacaaaggtgattgaagctagtctgaactaatcattagcaagtttgctaaaataaggttaagagggaaaactctctattcccaggaatctaagacatgatttaaagaaatcctcagagaacaaaaggctgtcaaatcaa

SEQ ID NO: 45 - DNA, 19 bp, Homo sapiens
agacagaggacatccacct

SEQ ID NO: 46 - DNA, 19 bp, Homo sapiens
atcgtgagccttcgtttgc

SEQ ID NO: 47 - DNA, 19 bp, Homo sapiens
ttatgggagagagttccct

SEQ ID NO: 48 - DNA, 19 bp, Homo sapiens
atatcataggtgatgggcc

SEQ ID NO: 49 - DNA, 19 bp, Homo sapiens
atgttacttgggttatggt

SEQ ID NO: 50 - DNA, 19 bp, Homo sapiens
ctgtgatgctctttcacac

SEQ ID NO: 51 - protein, 64 aa, Homo sapiens [sequence omitted]

SEQ ID NO: 52 - protein, 63 aa, Homo sapiens [sequence omitted]

SEQ ID NO: 53 - protein, 42 aa, Homo sapiens [sequence omitted]

SEQ ID NO: 54 - protein, 98 aa, Homo sapiens
MDQRLGHCQLSAHILHITHLQVLKTGKCPGAGRCCKHVSVPGGGAPAQLIGDSFYPAHPFGGYTSLAQSLIAEEHPPANSAQSPPPVLTTFHWFVCLN

SEQ ID NO: 55 - protein, 62 aa, Homo sapiens
AAPGAVPRSRLFEVRSALSVAKQITASTLLSVFSFVVLTSVAHLDLGCYYIFVFIINSCTCF

SEQ ID NO: 56 - protein, 52 aa, Homo sapiens [sequence omitted]

SEQ ID NO: 57 - protein, 33 aa, Homo sapiens [sequence omitted]

SEQ ID NO: 58 - protein, 103 aa, Homo sapiens
MSAEFFKKISQFASAFGWTLAPKAYRTLNPPDAPACLHLSPILRAPPPTLSSIANPLILIDGAFNPLCGPPRPSLEITGFHPPIPFPHTLHGAGSPRPPFLHS

SEQ ID NO: 59 - DNA, 4436 bp, Homo sapiens
cccccttgtcattaatacattaaaaagattcaatctttaccctgaggtaattttggccagttggtaccggatttataccaaaataatggacttgattggtattcaaaccaagatatgttggacggttaccagaggagaaggactcagtcctattgaaagctgtgaaggattgggagatcctgcttgcttttatgttgctgtaatttttattttaaatggactaatgatggcattattcttcatatatggcacatatttaagtggcagccgattaggaggcctggttacagtgttgtgcttctttttcaatcatggagagtgtacccgtgtaatgtggacaccacctctccgtgaaagcttctcatatccatttcttgttcttcagatgttgctagtgactcatattctcagggctacaaaactttatagaggaagcttgattgcactctgcatttccaatgtatttttcatgcttccttggcagtttgctcagtttgtacttcttactcagattgcatcattatttgcagtatatgttgtcgggtacattgatatatgtaaattacggaagatcatttatatacacatgatttctcttgcactttgttttgttttgatgtttgggaactcaatgttattaacttcttattatgcttcttctttggtaattatttggggtattctggcaatgaaaccacatttcctgaaaataaatgtatctgaacttagtttatgggttattcaaggatgtttttggttatttggaactgtcatacttaaatacttgacatctaaaatttttggtattgcagatgacgctcatattggcaacttactaacatcaaaattctttagttataaggattttgatactttattgtatacctgtgcagcggagtttgactttatggaaaaagagactccactgagatacacaaagacattattgcttccagttgttcttgtagtgtttgttgctattgttagaaagattattagtgatatgtggggtgtcttagctaaacaacagacacatgtaagaaaacaccagtttgatcatggagagctggtttaccatgcattgcaattgttagcatatacagcccttggtattttaattatgagactaaaactcttcttgacaccacacatgtgtgttatggcatcactgatctgctcaagacagctatttggatggctcttttgcaaagtacatcctggtgctattgtgtttgctatattagcagcaatgtcaatacaaggttcagcaaatctgcaaacccagtggaatattgtaggggagttcagcaatttgccccaagaagaacttatagaatggatcaaatatagtactaaaccagatgcagtgtttgcgggtgccatgcccacgatggcaagtgttaagctctctgcacttcggcccattgtgaatcatccacattatgaagacgcaggcttgagagccagaacaaaaatagtatactcaatgtatagtcggaaagcagccgaagaagtgaagcgagaactgataaagttaaaagtgaactattacattctagaagagtcatggtgtgtaagaagatccaagcctggttgcagtatgcctgaaatttgggatgtagaagatcctgccaatgctgggaaaactcccttatgtaacctcttggtgaaggattccaaacctcacttcaccactgtattccagaacagtgtttacaaagtcctagaagttgtaaaagaatgactgctacatgacctgctgcctacggagaactacatctgtaatggttttaatgttttgctaagtcatgtgttgttcatatcccaaaaacttttataggtaactgttttcaaatagaaaacgttttatttggtcaatttgaatgtcattctaattataaaaatgacttacacctttatcaattggttactatttcaatgcaccctttaaaatttgctatgcaaatgagtatatgcttgtacttgactttaatatttgtgctaaagtgagcaaagctacctgtataaagaaaacacagtgggttgtgacaaggatgacatgaaaatacaggacaattctgacaatgtaggggctgattttatagtgtaagaactattaatgccccttgcttcttttttctgcctcttgctcttgtcttttggacatttcagtgattgtaagttcttcggtcatgtcagcccctgtcatcaacttgagttacagtagatggggcagacatggagtgtttgctatataaaactatctgtttgttttacttccttgtgcgctttttgttctctgttctcttgttaatgaagcttttcctgcccattattaatccaaactcttggaccttgtggttaggaaattcccttaacttccagccatatggcattatcgtgtctctttctctctctctcttgctctctctcttctcctcttccccatattttctgtcaaataagtactgtttactcatttagttgcttatcaagtacttattcttggttttaaaaaaaattaatggtaactgtatttttctcatttttagcattattcaaatgtttatattttaatacctttaaaccactttaaagttttttcatgtttaattatagttttaagaaaaactattttgaacaaccccaaatatagtgcatctagaaactaatgtatatttgattagacatcatttatagtggaacagtagactgtagtacatggtaatttttcttttactattaagatacaataaaacatgactaattttgctgtcaaaaatgtaaagaataatgataaatggagttttttatattttacttttaagattgcctgtctttaataagacaaagccttaagccttatgttataattttggttctaaaaaccatcatttcagtataaggaataagtatatttcgtcctcctctttagtttttttcttcctatttatttttattttgaaaaatttctacaccttctttgaattccttgtatgaatttttgtttcttagaagttaatttgtgtgaaatgagattcttcaaaacgatgaaacctcatagctctgagaaaaggttttagggttttaaattctaagcaaagcgtgactatggctgacagactacacatttaattatacagcttctctttcttaaccacaggcagattaacctcattgtggattgtccttcagaccttagtcctcaggcatggtttctggtgcccactcctggaagccgctgttccctttctaccttcttaccagagcccaagggcaggcctggtcccggggaagcagcagcttgctgacataagtcagctgcaaaggctgaggagtgtgccctcagagaagcaccgccccccagtcttgtgccagcgcctagagccgcagctcccagggatgctccttccctggaggcagcccaggagagggactctggcagcgttcttcagatttgtggccactgtttctcatttgctggttgactgtttttatttcttaggcttttgctagttttagaaaatagggaagcagcccttgatttgtggattaaaagcaacatttgagcgatgatgcacaacagtccaggaaaatgggcggtggacacttgaggctgaggatgggagttgacatgagcagggagagggaggtgcgcgctgcttatctgtgattgttgctcacctgagtgtggctgattgtgtacatccagcagttacaatttttaaaaattatacttttacatttattttatatttttctcacccccagtaatttccttccaaagaagttcacatgtaataagtagaaattctgtataggaaaaaagcattaaaaatactattataactgcttcatttgctgggaaccattaaaagtaatataaattagctttttccagaaggatccttttgtagcagtgtttatgaatgtaacccccagcaaaatatggctatatattaggggagccagtttggagcagaggcctgaaggtccctgctatgcagccgtggccacagctcgcagcccaagcactgtggagcatccacacctttgatggcaatgcagattggtagcaggttccataggcgtacaaaacagtattaaagctcagtgttttgcatattgttagcatttacaaatatttttgctttagtatgaggaaagtaaggatgggcaaagaagcgatcaaaatagctattgctacaacattttcgaaaacaaagttggggctgtatttctttaaaaagataagcctctaaaaatgcttggcaaaaaaaatatagtgttaaaataggccagtgatattaatgagaaaatgaaagtatgtatcaggaataaagtgatattgcataggagtattgtatttttatgaattttatgccagttgtttacatgtactatatatgttaaattaaaaaaaatcatgagaaatg

SEQ ID NO: 60 - protein, 580 aa, Homo sapiens
PLVINTLKRFNLYPEVILASWYRIYTKIMDLIGIQTKICWTVTRGEGLSPIESCEGLGDPACFYVAVIFILNGLMMALFFIYGTYLSGSRLGGLVTVLCFFFNHGECTRVMWTPPLRESFSYPFLVLQMLLVTHILRATKLYRGSLIALCISNVFFMLPWQFAQFVLLTQIASLFAVYVVGYIDICKLRKIIYIHMISLALCFVLMFGNSMLLTSYYASSLVIIWGILAMKPHFLKINVSELSLWVIQGCFWLFGTVILKYLTSKIFGIADDAHIGNLLTSKFFSYKDFDTLLYTCAAEFDFMEKETPLRYTKTLLLPVVLVVFVAIVRKIISDMWGVLAKQQTHVRKHQFDHGELVYHALQLLAYTALGILIMRLKLFLTPHMCVMASLICSRQLFGWLFCKVHPGAIVFAILAAMSIQGSANLQTQWNIVGEFSNLPQEELIEWIKYSTKPDAVFAGAMPTMASVKLSALRPIVNHPHYEDAGLRARTKIVYSMYSRKAAEEVKRELIKLKVNYYILEESWCVRRSKPGCSMPEIWDVEDPANAGKTPLCNLLVKDSKPHFTTVFQNSVYKVLEVVKE

(19) United States
(12) Patent Application Publication
(10) Pub. No.: US 2011/0064730 A1
(43) Pub. Date: Mar. 17, 2011

(54) METHOD OF MODULATING ANGIOGENESIS

(76) Inventors: Thomas John Gonda, Chapel Hill (AU); Gabriel Kremmidiotis, Flagstaff Hill (AU)

(21) Appl. No.: 12/861,624

(22) Filed: Aug. 23, 2010

Related U.S. Application Data
(63) Continuation-in-part of application No. 10/550,533, filed on Apr. 28, 2006, now abandoned.

(30) Foreign Application Priority Data
Mar. 28, 2003 (AU) 2003901511

(51) Int. Cl. A61K 39/395; C12N 5/00; A61K 31/7105; C12Q 1/02

(52) U.S. Cl. 424/133.1; 435/375; 514/44 A; 424/174.1; 435/29

(57) ABSTRACT

A method for the identification of a nucleic acid molecule differentially expressed in an in vitro model of a biological system, comprising the steps of: (1) harvesting cells from the model system at predetermined time points; (2) obtaining total RNA from the cells harvested at each time point; (3) preparing cDNA from the total RNA from each time point to provide a plurality of pools of cDNA; (4) performing a suppression subtractive hybridization (SSH) on the cDNA pools from each time point sequentially so as to progressively amplify cDNAs derived from nucleic acid molecules differentially expressed from one time period to the next.

### RELATED APPLICATIONS

This application is a continuation-in-part patent application which claims the benefit of the filing date of U.S. patent application Ser. No. 10/550,533, filed Sep. 22, 2005, which claims the benefit of the filing date of PCT International Patent Application Serial No. PCT/AU2004/000383, filed Mar. 26, 2004, which claims the benefit of the filing date of Australian Patent Application Serial No. 2003901511, filed Mar. 28, 2003, the contents of each of which are herein incorporated by reference in their entirety.

### TECHNICAL FIELD

The present invention relates to novel nucleic acid sequences (“angiogenic genes”) involved in the process of angiogenesis. Each of the angiogenic genes encode a polypeptide that has a role in angiogenesis. In view of the realisation that these genes play a role in angiogenesis, the invention is also concerned with the therapy of pathologies associated with angiogenesis, the screening of drugs for pro- or anti-angiogenic activity, the diagnosis and prognosis of pathologies associated with angiogenesis, and in some cases the use of the nucleic acid sequences to identify and obtain full-length angiogenesis-related genes.

### BACKGROUND

The formation of new blood vessels from pre-existing vessels, a process termed angiogenesis, is essential for normal growth. Important angiogenic processes include those taking place in embryogenesis, renewal of the endometrium, formation and growth of the corpus luteum of pregnancy, wound healing and in the restoration of tissue structure and function after injury.

The formation of new capillaries requires a co-ordinated series of events mediated through the expression of multiple genes which may have either pro- or anti-angiogenic activities. The process begins with an angiogenic stimulus to existing vasculature, usually mediated by growth factors such as vascular endothelial growth factor or basic fibroblast growth factor. This is followed by degradation of the extracellular matrix, cell adhesion changes (and disruption), an increase in cell permeability, proliferation of endothelial cells (ECs) and migration of ECs towards the site of blood vessel formation. Subsequent processes include capillary tube or lumen formation, stabilisation and differentiation by the migrating ECs.

In the (normal) healthy adult, angiogenesis is virtually arrested and occurs only when needed. However, a number of pathological situations are characterised by enhanced, uncontrolled angiogenesis. These conditions include cancer, rheumatoid arthritis, diabetic retinopathy, psoriasis and cardiovascular diseases such as atherosclerosis. In other pathologies such as ischaemic limb disease or in coronary artery disease, growing new vessels through the promotion of an expanding vasculature would be of benefit.

A number of in vitro assays have been established which are thought to mimic angiogenesis and these have provided important tools to examine the mechanisms by which the angiogenic process takes place and the genes most likely to be involved.

Lumen formation is a key step in angiogenesis. The presence of vacuoles within ECs undergoing angiogenesis have been reported and their involvement in lumen formation has been postulated (Folkman and Haudenschild, 1980; Gamble et al., 1993). The general mechanism of lumen formation suggested by Folkman and Haudenschild (1980), has been that vacuoles form within the cytoplasm of a number of aligned ECs which are later converted to a tube. The union of adjacent tubes results in the formation of a continuous unicellular capillary lumen. However, little is known about the changes in cell morphology leading to lumen formation or the signals required for ECs to construct this feature.

An in vitro model of angiogenesis has been created from human umbilical vein ECs plated onto a 3 dimensional collagen matrix (Gamble et al., 1993). In the presence of phorbol myristate acetate (PMA) these cells form capillary tubes within 24 hours. With the addition of anti-integrin antibodies, the usually unicellular tubes (thought to reflect an immature, poorly differentiated phenotype) are converted to form a multicellular lumen through the inhibition of cell-matrix interactions and promotion of cell-cell interactions. This model has subsequently allowed the investigation of the morphological events which occur in lumen formation.

For the treatment of diseases associated with angiogenesis, understanding the molecular genetic mechanisms of the process is of paramount importance. The use of the in vitro model described above (Gamble et al., 1993), a model that reflects the critical events that occur during angiogenesis in vivo in a time dependant and broadly synchronous manner, has provided a tool for the identification of the key genes involved.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method of modulating angiogenesis comprising modulating the expression or activity of a BNO802 polypeptide in a cell, wherein the BNO802 polypeptide is encoded by a BNO802 nucleic acid molecule set forth in Table 1. In some embodiments, the expression or activity of the BNO802 polypeptide is modulated by introducing into the cell an antisense to the BNO802 nucleic acid molecule. In some embodiments, the expression or activity of the BNO802 polypeptide is modulated by introducing into the cell a nucleic acid which is an siRNA. In some embodiments, the expression or activity of the BNO802 polypeptide is modulated by an antibody capable of binding the BNO802 polypeptide. In some embodiments, the antibody is a fully human antibody. In some embodiments, the antibody is selected from the group consisting of a monoclonal antibody, a humanised antibody, a chimaeric antibody or an antibody fragment including a Fab fragment, (Fab′)2 fragment, Fv fragment, single chain antibodies and single domain antibodies.

In some embodiments, the presently disclosed subject matter provides a method for the treatment of an angiogenesis-related disorder, comprising modulating the expression or activity of a BNO802 polypeptide encoded by a BNO802 nucleic acid molecule set forth in Table 1. In some embodiments, the expression or activity of the BNO802 polypeptide is modulated by introducing into the cell an antisense to the BNO802 nucleic acid molecule. In some embodiments, the expression or activity of the BNO802 polypeptide is modulated by introducing into the cell a nucleic acid which is an siRNA. In some embodiments, the expression or activity of the BNO802 polypeptide is modulated by an antibody capable of binding the BNO802 polypeptide. In some embodiments, the antibody is a fully human antibody. In some embodiments, the antibody is selected from the group consisting of a monoclonal antibody, a humanised antibody, a chimaeric antibody or an antibody fragment including a Fab fragment, (Fab′)2 fragment, Fv fragment, single chain antibodies and single domain antibodies. In some embodiments, the disorder is selected from the group consisting of cancer, rheumatoid arthritis, diabetic retinopathy, psoriasis, and cardiovascular diseases such as atherosclerosis, ischaemic limb disease or coronary artery disease.

In some embodiments, the presently disclosed subject matter provides a method of screening for a candidate pharmaceutical compound for the treatment of an angiogenesis-related disorder, comprising the steps of: (1) providing a BNO802 polypeptide set forth in Table 1; (2) adding a candidate pharmaceutical compound to said BNO802 polypeptide; and (3) determining the binding of said candidate compound to said BNO802 polypeptide; wherein a compound that binds to the polypeptide is a candidate for the treatment of an angiogenesis-related disorder.

In some embodiments, the presently disclosed subject matter provides a method of screening for a candidate pharmaceutical compound useful in the treatment of an angiogenesis-related disorder, comprising the steps of: (1) providing a cell transformed with an expression vector comprising a BNO802 nucleic acid molecule set forth in Table 1; (2) adding a candidate pharmaceutical compound to said cell; and (3) determining the effect of said candidate pharmaceutical compound on the expression or activity of the polypeptide encoded by the BNO802 nucleic acid molecule that is part of the expression vector in said cell; wherein a compound that alters the expression or activity of the polypeptide encoded by the BNO802 nucleic acid molecule that is part of the expression vector in said cell is a candidate for the treatment of an angiogenesis-related disorder.

It is an object of the presently disclosed subject matter to provide methods of modulating angiogenesis comprising modulating the expression or activity of a BNO802 polypeptide in a cell.

An object of the presently disclosed subject matter having been stated above, other objects and advantages will become apparent to those of ordinary skill in the art after a study of the following description of the presently disclosed subject matter and non-limiting Examples.

### BRIEF DESCRIPTION OF THE DRAWINGS

FIGS. 1A-1D. Example of the expression profile of selected differentially expressed clones during defined time points in the in vitro model of angiogenesis. Time points at the defined stages of 0.5 hours, 3 hours, 6 hours and 24 hours of the in vitro tube formation assay were plotted against the log ratio of cy5 (red) and cy3 (green) dyes used for microarray hybridizations. FIG. 1A: example of a clone with peak expression at the 0.5 hour time point; FIG. 1B: example of a clone with peak expression at the 3 hour time point; FIG. 1C: example of a clone with peak expression at the 6 hour time point; and FIG. 1D: example of a clone with peak expression at the 24 hour time point.

FIGS. 2A and 2B. Expression profile of differentially expressed genes BNO782 and BNO481. Both genes show peak expression at the 6 hour time point of the in vitro tube formation assay. FIG. 2A: BNO782; FIG. 2B: BNO481.

FIGS. 3A and 3B. Analysis of the level of BNO782 expression knock-down mediated by BNO782 siRNA2 and BNO481 expression knock-down mediated by BNO481 siRNA1, as measured by real-time RT-PCR. The three siRNA oligonucleotides targeted to each gene were able to reduce expression of the gene to varying degrees with BNO781 siRNA2 inhibiting BNO781 expression by 24% (FIG. 3A) and BNO481 siRNA1 inhibiting expression of BNO481 by 36% (FIG. 3B).

FIGS. 4A-4D. Reducing BNO782 or BNO481 mRNA expression inhibits HUVEC tube formation. HUVECs infected with BNO782 siRNA2, BNO481 siRNA1, or a vector control were plated on Matrigel for 24 hrs. Vector infected cells formed extensive networks of tube structures (FIGS. 4A and 4C). In contrast, cells infected with BNO782 siRNA2 or BNO481 siRNA1 exhibited tube structure networks of significantly reduced complexity with a high number of incomplete tube extensions (FIGS. 4B and 4D).

FIGS. 5A-5C. Evaluation of the consequences of siRNA-mediated knockdown of BNO802 on the formation of capillary tubes by endothelial cells on Matrigel. Pictures of endothelial capillary tubes were taken after 22 hours incubation using a Olympus BX51 microscope with 4× objective and CCD Optronics high resolution camera and Olympus CKX41 inverted microscope with DP11 digital camera.

FIGS. 6A and 6B. Evaluation of the consequences of siRNA-mediated knockdown of BNO802 on the ability of endothelial cells to proliferate. Endothelial cells are seeded at 1000 cells/well and cultured for 72 hours. Cell growth was evaluated using a colorimetric assay. Error bars are base on standard deviation derived from triplicate wells.

FIG. 7. RealTime-RTPCR analysis evaluating the degree of BNO802 gene knockdown achieved with RNAi. Total RNA was extracted from cells and reverse transcribed into cDNA followed by RealTime PCR amplification using gene specific primers. Expression levels were normalised to the house-keeping gene POLR2K and expressed as a percent of the vector control (n=3).

FIG. 8. Evaluation of BNO802 gene expression in normal human tissues using RealTime RTPCR analysis. Human RNA samples (Ambion) were reverse transcribed into cDNA followed by RealTime PCR using gene specific primers. Gene expression data was normalised to the expression of the house-keeping gene POLR2K. The level of gene expression in each tissue was expressed relative to the gene expression found in a homogeneous endothelial cell population (HUVEC) (n=4).

### DETAILED DESCRIPTION

Total RNA from cells harvested at specific time points from a biological model, in this case the Gamble et al (1993) model for angiogenesis, were used to prepare cDNAs, which were subjected to a novel process incorporating suppression subtractive hybridization (SSH) to identify cDNAs derived from differentially expressed genes.

According to one aspect of the present invention there is provided a method for the identification of a gene differentially expressed in an in vitro model of a biological system, comprising the steps of:

(1) harvesting cells from the model system at predetermined time points;

(2) obtaining total RNA from the cells harvested at each time point;

(3) preparing cDNA from the total RNA from each time point to provide a plurality of pools of cDNA;

(4) performing a suppression subtractive hybridization (SSH) on the cDNA pools from each time point sequentially so as to progressively amplify cDNAs derived from genes differentially expressed from one time period to the next.

Thus, up-regulation of a gene whose expression subsequently remains up-regulated at the same level will be detected (and the cDNA amplified) only in the first time period where the level cDNA is elevated, as the quantity of cDNA in pools is from the subsequent time points will be the same. This reduction in redundancy reduces the possibility that other genes of lower representation in the cell mRNA expression pool will be masked. In a particularly preferred embodiment of the present invention the model system is an in vitro model for angiogenesis (Gamble et al., 1993).

Those cDNAs identified to be differentially expressed in the SSH process were cloned and subjected to microarray analysis, which lead to the identification of a number of genes that are up-regulated in their expression during the angiogenesis process.

According to a further aspect of the present invention there is provided a method for the identification of a gene up-regulated in an in vitro model of a biological system, comprising the steps of:

(1) harvesting cells from the model system at predetermined time points;

(2) obtaining total RNA from the cells harvested at each time point;

(3) preparing cDNA from the total RNA from each time point to provide a plurality of pools of cDNA;

(4) performing a suppression subtractive hybridization (SSH) on the cDNA pools from each time point sequentially so as to progressively amplify cDNAs derived from genes differentially expressed from one time period to the next.

(5) cloning the amplified cDNAs;

(6) locating DNA from each clone on a microarray;

(7) generating antisense RNA by reverse transcription of total RNA from cells harvested from the in vitro model at said predetermined time intervals and labelling the antisense RNA; and

(8) probing the microarray with labelled antisense RNA from 0 hours and each of the other time points separately to identify clones containing cDNA derived from genes which are up-regulated at said time points in the in vitro model.

Functional analysis of a subset of these up-regulated angiogenic genes and their effect on endothelial cell function and capillary tube formation is described in detail below.

Accordingly, the present invention provides isolated nucleic acid molecules, which have been shown to be up-regulated in their expression during angiogenesis (see Tables 1 and 2). The isolation of these angiogenic genes has provided novel targets for the treatment of angiogenesis-related disorders.

In a first aspect of the present invention there is provided an isolated nucleic acid molecule as defined by SEQ ID Numbers: 1 to 44.

Following the realisation that these molecules, and those listed in Tables 1 and 2, are up-regulated in their expression during angiogenesis, the invention provides isolated nucleic acid molecules as defined by SEQ ID Numbers: 1 to 44, and laid out in Tables 1 and 2, or fragments thereof, that play a role in an angiogenic process. Such a process may include, but is not restricted to, embryogenesis, menstrual cycle, wound repair, tumour angiogenesis and exercise induced muscle hypertrophy.

In addition, the present invention provides isolated nucleic acid molecules as defined by SEQ ID Numbers: 1 to 44, and laid out in Tables 1 and 2 (hereinafter referred to as “angiogenic genes”, “angiogenic nucleic acid molecules” or “angiogenic polypeptides” for the sake of convenience), or fragments thereof, that play a role in diseases associated with the angiogenic process. Diseases may include, but are not restricted to, cancer, rheumatoid arthritis, diabetic retinopathy, psoriasis, and cardiovascular diseases such as atherosclerosis, ischaemic limb disease and coronary artery disease. Useful fragments may include those which are unique and which do not overlap any previously identified genes, unique fragments which do overlap with a known sequence, and fragments which span alternative splice junctions etc.

TABLE 1
Novel Angiogenesis Genes

| | | | | | Peak |
|---|---|---|---|---|---|
| BNO | | | UniGene | | Expression |
| Number | Symbol | Gene Description - Homology | Number | GenBank Number | (h) |
| BNO605 | BNO605 | EST, UI-HF-BR0p-ajy-c-08-0-UI.s1 Homo sapiens cDNA | None | AW576601 | |
| BNO612 | FLJ20445 | hypothetical protein FLJ20445 | Hs.343748 | NM_017824 | 6 |
| BNO616 | MGC2747 | hypothetical protein MGC2747 | Hs.194017 | NM_024104 | 0.5, 6 |
| BNO617 | FLJ20986 | hypothetical protein FLJ20986 | Hs.324507 | NM_024524 | 6 |
| BNO618 | FLJ14834 | hypothetical protein FLJ14834 | Hs.62905 | NM_032849 | 3 |
| BNO620 | FLJ22746 | hypothetical protein FLJ22746 | Hs.147585 | NM_024785 | 0.5 |
| BNO622 | KIAA1376 | KIAA1376 protein | Hs.24684 | BC015928 | 3, 24 |
| BNO627 | BNO627 | EST, AV756199 BM Homo sapiens cDNA clone | None | SEQ ID NO: 1 | 6 |
| | | BMFAUH02 5′ | | | |
| BNO628 | BNO628 | EST, QV1-BT0631-130300-111-e03 BT0631 Homo | None | SEQ ID NO: 2 | 6 |
| | | sapiens cDNA | | | |
| BNO629 | BNO629 | EST, Homo sapiens cDNA clone IMAGE: 2664022 3′ | None | SEQ ID NO: 3 | 6 |
| BNO630 | BNO630 | EST, Homo sapiens cDNA clone IMAGE: 2357465 3′ | None | SEQ ID NO: 4, 51 | 6 |
| BNO632 | BNO632 | ESTs | Hs.404198 | SEQ ID NO: 5 | 6 |
| BNO633 | BNO633 | ESTs, Weakly similar to hypothetical protein FLJ20378 | Hs.310598 | SEQ ID NO: 6 | 24 |
| BNO634 | BNO634 | ESTs | Hs.345443 | SEQ ID NO: 7 | 6 |
| BNO635 | BNO635 | Hypothetical protein | Hs.54347 | BC057847 | 6 |
| BNO636 | BNO636 | ESTs | Hs.105636 | SEQ ID NO: 8 | 3 |
| BNO637 | BNO637 | ESTs | Hs.486928 | SEQ ID NO: 9, 52 | 6 |
| BNO638 | BNO638 | EST | None | SEQ ID NO: 10 | 6 |
| BNO639 | BNO639 | None | None | SEQ ID NO: 11, 53 | 6 |
| BNO640 | BNO640 | None | None | SEQ ID NO: 12 | 6 |
| BNO645 | FLJ10498 | hypothetical protein FLJ10498 | Hs.270107 | NM_018115 | 24 |
| BNO648 | LOC57146 | hypothetical protein from clone 24796 | Hs.27191 | NM_020422 | 0.5 |
| BNO652 | FLJ31051 | hypothetical protein FLJ31051 | Hs.406199 | NM_153687 | 6 |
| BNO655 | LOC51122 | HSPC042 protei | Hs.432729 | NM_016094 | 3 |
| BNO659 | FLJ32123 | FLJ32123 | Hs.349397 | AK056685 | 6 |
| BNO662 | BNO662 | ESTs | Hs.444495 | BX647355 | 6 |
| BNO664 | FLJ10312 | FLJ10312 | None | NM_030672 | 3 |
| BNO669 | BNO669 | ESTs | Hs.172998 | BC030094 | 3 |
| BNO671 | KIAA0882 | KIAA0882 protein | Hs.411317 | AB020689 | 3 |
| BNO673 | BNO673 | hypothetical protein DKFZp434L142 | Hs.323583 | NM_016613 | 6 |
| BNO675 | FLJ10700 | hypothetical protein FLJ10700 | Hs.295909 | NM_018182 | 3 |
| BNO677 | FLJ30135 | FLJ30135 | Hs.34906 | BC020494 | 3, 24 |
| BNO685 | FLJ10849 | hypothetical protein FLJ10849 | Hs.386784 | NM_018243 | |
| BNO687 | MGC45416 | hypothetical protein MGC45416 | Hs.95835 | NM_152398 | 24 |
| BNO690 | C15orf15 | chromosome 15 open reading frame 15 | Hs.274772 | NM_016304 | 3 |
| BNO694 | BNO694 | cDNA DKFZp566E0124 | None | AL050030 | 6 |
| BNO697 | BNO697 | Hypothetical protein MGC45871 | Hs.345588 | BC014203 | 24 |
| BNO700 | C7orf30 | chromosome 7 open reading frame 30 | Hs.87385 | NM_138446 | 24 |
| BNO704 | KIAA1102 | KIAA1102 protein | Hs.156761 | AB029025 | |
| BNO705 | BNO705 | ESTs | Hs.30280 | SEQ ID NO: 13 | 3 |
| BNO706 | LOC116441 | hypothetical protein BC014339 | Hs.22026 | NM_138786 | 24 |
| BNO708 | BNO708 | ESTs | Hs.12876 | SEQ ID NO: 14 | 6 |
| BNO710 | BNO710 | FLJ23228 | Hs.170623 | AK026881 | 6 |
| BNO712 | BNO712 | FLJ21592 | Hs.5921 | AK025245 | 3 |
| BNO713 | KIAA0970 | KIAA0970 protein | Hs.103329 | NM_014923 | 6 |
| BNO714 | KIAA0121 | KIAA0121 gene product | Hs.155584 | D50911 | 6 |
| BNO723 | C14orf123 | chromosome 14 open reading frame 123 | Hs.279761 | NM_014169 | 6 |
| BNO725 | KIAA0582 | KIAA0582 protein | Hs.146007 | NM_015147 | 24 |
| BNO730 | BNO730 | ESTs | Hs.158753 | SEQ ID NO: 15 | 6 |
| BNO731 | C6orf166 | chromosome 6 open reading frame 166 | Hs.201864 | NM_018064 | 3 |
| BNO735 | FLJ32029 | Unnamed protein product | Hs.26612 | NM_173582 | 6 |
| BNO737 | BNO737 | hypothetical protein DKFZp434F0318 | Hs.23388 | NM_030817 | |
| BNO740 | KIAA1728 | KIAA1728 protein | Hs.437362 | AB051515 | 24 |
| BNO742 | BNO742 | hypothetical protein FLJ11795 | Hs.84560 | NM_024669 | 24 |
| BNO745 | BNO745 | hypothetical protein DKFZp547A023 | Hs.374649 | NM_018704 | 6 |
| BNO747 | MGC23937 | hypothetical protein MGC23937 similar to CG4798 | Hs.91612 | NM_145052 | 6 |
| BNO753 | BNO753 | cDNA DKFZp667P1024 | Hs.127811 | AL832835 | 3 |
| BNO754 | KIAA0303 | KIAA0303 protein | Hs.212787 | AB002301 | 3 |
| BNO756 | BNO756 | ESTs | Hs.443155 | SEQ ID NO: 16, 54 | |
| BNO759 | KIAA1416 | KIAA1416 protein | Hs.397426 | AB037837 | 6 |
| BNO761 | C7orf24 | chromosome 7 open reading frame 24 | Hs.444840 | NM_024051 | 6 |
| BNO762 | FLJ11223 | cDNA FLJ11223 | Hs.92308 | AL832083 | 3 |
| BNO768 | FLJ30478 | cDNA FLJ30478 | Hs.298258 | AK092048 | 6 |
| BNO772 | FLJ10525 | Hypothetical protein FLJ10525 | Hs.31082 | NM_018126 | 6 |
| BNO780 | LOC58489 | hypothetical protein from EUROIMAGE 588495 | Hs.26765 | AL390079 | 3 |
| BNO782 | MGC26717 | Hypothetical protein | Hs.406060 | BC024188 | 6 |
| BNO791 | KIAA1053 | KIAA1053 protein | Hs.98259 | NM_015589 | 6 |
| BNO793 | KIAA0766 | KIAA0766 gene product | Hs.28020 | NM_014805 | 24 |
| BNO795 | BNO795 | ESTs moderately similar to MDC-3.13 isoform 2 mRNA | Hs.306343 | AK123281 | 6 |
| BNO800 | KIAA1577 | KIAA1577 protein | Hs.449290 | AB046797 | 6 |
| BNO802 | KIAA0877 | KIAA0877 protein | Hs.408623 | SEQ ID NO: 59, 60 | 24 |
| BNO812 | KIAA0372 | KIAA0372 gene product | Hs.435330 | NM_014639 | 6 |
| BNO816 | BNO816 | cDNA clone 4052238 | Hs.348514 | BC014384 | 6 |
| BNO818 | MGC10067 | hypothetical protein MGC10067 | Hs.42251 | NM_145049 | 3 |
| BNO819 | KIAA1191 | KIAA1191 protein | Hs.8594 | NM_020444 | 24 |
| BNO821 | BNO821 | ESTs | Hs.87606 | SEQ ID NO: 17 | 24 |
| BNO825 | FBXO30 | F-box protein 30 | Hs.421095 | NM_032145 | 3 |
| BNO831 | C8orf1 | chromosome 8 open reading frame 1 | Hs.436445 | NM_004337 | 24 |
| BNO833 | C6orf115 | Chromosome 6 open reading frame 115 | Hs.238205 | BC014953 | 24 |
| BNO838 | BNO838 | ESTs | Hs.319095 | SEQ ID NO: 18 | 3 |
| BNO845 | FLJ23728 | cDNA FLJ23728 | Hs.191094 | AK074308 | 6 |
| BNO848 | C10orf45 | Chromosome 10 open reading frame 45 | Hs.103378 | NM_031453 | 24 |
| BNO849 | BNO849 | cDNA DKFZp434G0972 | Hs.106148 | AL133577 | 24 |
| BNO852 | CGI-111 | CGI-111 protein | Hs.11085 | NM_016048 | 6 |
| BNO856 | LOC116068 | hypothetical protein LOC116068 | Hs.136235 | AL832721 | 24 |
| BNO857 | C12orf2 | chromosome 12 open reading frame 2 | Hs.140821 | NM_007211 | 6 |
| BNO862 | BNO862 | DKFZP434C212 protein | Hs.287266 | AK023841 | |
| BNO868 | BNO868 | DKFZP566C134 protein | Hs.20237 | AB040922 | 3 |
| BNO870 | LOC57228 | hypothetical protein from clone 643 | Hs.206501 | NM_020467 | 24 |
| BNO871 | KIAA1463 | KIAA1463 protein | Hs.21104 | AB040896 | 6 |
| BNO873 | KIAA1376 | KIAA1376 protein | Hs.24684 | NM_020801 | 0.5, 24 |
| BNO876 | FLJ10326 | hypothetical protein FLJ10326 | Hs.262823 | NM_018060 | 24 |
| BNO878 | BNO878 | hypothetical protein DKFZp761L1417 | Hs.270753 | NM_152913 | 6 |
| BNO881 | MGC11349 | hypothetical protein MGC11349 | Hs.288697 | NM_025112 | 6 |
| BNO883 | FLJ39541 | similar to RIKEN cDNA 9130404H11 gene | Hs.21388 | NM_178566 | 6 |
| BNO886 | BNO886 | cDNA DKFZp686D04119 | Hs.30258 | BX537597 | 6 |
| BNO887 | KIAA0648 | KIAA0648 protein | Hs.31921 | NM_015200 | 24 |
| BNO890 | KIAA1160 | KIAA1160 protein | Hs.512661 | NM_020701 | 3 |
| BNO892 | C20orf108 | chromosome 20 open reading frame 108 | Hs.143736 | NM_080821 | 3 |
| BNO894 | KIAA0205 | KIAA0205 gene product | Hs.528724 | NM_014873 | 6 |
| BNO895 | C20orf112 | chromosome 20 open reading frame 112 | Hs.335142 | NM_080616 | 0.5 |
| BNO898 | BNO898 | clone IMAGE: 5243590 | Hs.454832 | BC036880 | 6 |
| BNO905 | KIAA1462 | KIAA1462 protein | Hs.192726 | AB040895 | 3 |
| BNO906 | KIAA1199 | KIAA1199 protein | Hs.212584 | AB033025 | 6 |
| BNO908 | C15orf12 | chromosome 15 open reading frame 12 | Hs.513041 | NM_018285 | |
| BNO910 | BNO910 | cDNA DKFZp564F053 | Hs.529772 | AL049265 | 6 |
| BNO917 | BNO917 | hypothetical protein dJ465N24.2.1 | Hs.259412 | NM_020317 | 24 |
| BNO926 | KIAA1238 | KIAA1238 protein | Hs.372288 | AB033064 | |
| BNO928 | BNO928 | EST | None | SEQ ID NO: 19 | 3 |
| BNO929 | BNO929 | EST | None | SEQ ID NO: 20 | 6 |
| BNO930 | BNO930 | EST | Hs.478376 | SEQ ID NO: 21 | 6 |
| BNO932 | BNO932 | EST | Hs.492501 | SEQ ID NO: 22, 55 | 3 |
| BNO933 | BNO933 | EST | None | SEQ ID NO: 23 | 6 |
| BNO934 | BNO934 | EST | None | SEQ ID NO: 24 | 6 |
| BNO935 | BNO935 | EST | None | SEQ ID NO: 25 | 6 |
| BNO936 | BNO936 | EST | None | SEQ ID NO: 26, 56 | 6 |
| BNO937 | BNO937 | alpha gene sequence | None | AF203815 | 6 |
| BNO938 | BNO938 | EST | None | SEQ ID NO: 27 | 0.5 |
| BNO939 | BNO939 | EST | None | SEQ ID NO: 28 | 6 |
| BNO940 | BNO940 | EST | None | SEQ ID NO: 29 | 6 |
| BNO941 | BNO941 | EST | None | SEQ ID NO: 30 | 3 |
| BNO942 | BNO942 | EST | None | SEQ ID NO: 31 | 6 |
| BNO943 | BNO943 | EST | None | SEQ ID NO: 32 | 6 |
| BNO944 | BNO944 | EST | None | SEQ ID NO: 33 | 6 |
| BNO945 | BNO945 | EST | None | SEQ ID NO: 34 | 6 |
| BNO946 | BNO946 | EST | None | SEQ ID NO: 35, 57 | 6 |
| BNO948 | BNO948 | EST | None | SEQ ID NO: 36 | 6 |
| BNO949 | BNO949 | EST | None | SEQ ID NO: 37, 58 | 3 |
| BNO950 | BNO950 | EST | None | SEQ ID NO: 38 | 24 |
| BNO951 | BNO951 | EST | None | SEQ ID NO: 39 | 24 |
| BNO953 | BNO953 | EST | None | SEQ ID NO: 40 | 24 |
| BNO961 | BNO961 | FLJ00138 protein | Hs.199749 | AK074067 | 3, 24 |
| BNO1018 | BNO1018 | EST | Hs.485935 | SEQ ID NO: 41 | 3 |
| BNO1019 | BNO1019 | EST | None | SEQ ID NO: 42 | 24 |
| BNO1020 | BNO1020 | EST | None | SEQ ID NO: 43 | 3 |
| BNO1021 | BNO1021 | EST | None | SEQ ID NO: 44 | 3 |

TABLE 2
Genes with a Previously Unknown Role in Angiogenesis

| | | | | | Peak |
|---|---|---|---|---|---|
| BNO | | | UniGene | | Expression |
| Number | Symbol | Gene Description - Homology | Number | GenBank Number | (h) |
| BNO436 | NP | nucleoside phosphorylase | Hs.75514 | NM_000270 | 6 |
| BNO438 | CD59 | CD59 antigen p18-20 | Hs.278573 | NM_000611 | 24 |
| BNO441 | BIRC3 | baculoviral IAP repeat-containing 3 | Hs.127799 | NM_001165 | 3 |
| BNO442 | FABP5 | fatty acid binding protein 5 (psoriasis-associated) | Hs.408061 | NM_001444 | 24 |
| BNO443 | CBFB | core-binding factor, beta subunit | Hs.179881 | NM_001755 | 6 |
| BNO446 | INHBA | inhibin, beta A (activin A, activin AB alpha polypeptide) | Hs.727 | NM_002192 | 6 |
| BNO447 | MGST2 | microsomal glutathione S-transferase 2 | Hs.81874 | NM_002413 | 24 |
| BNO448 | RAB6A | RAB6A, member RAS oncogene family | Hs.5636 | NM_002869 | 6 |
| BNO449 | SAT | spermidine/spermine N1-acetyltransferase | Hs.28491 | NM_002970 | 6 |
| BNO451 | TXNRD1 | thioredoxin reductase 1 | Hs.13046 | NM_003330 | 6 |
| BNO452 | SLC4A7 | solute carrier family 4, sodium bicarbonate cotransporter, | Hs.132904 | NM_003615 | 6 |
| | | member 7 | | | |
| BNO453 | PPAP2B | phosphatidic acid phosphatase type 2B | Hs.432840 | NM_003713 | 3 |
| BNO454 | BCL10 | B-cell CLL/lymphoma 10 | Hs.193516 | NM_003921 | 3 |
| BNO455 | DUSP1 | dual specificity phosphatase 1 | Hs.171695 | NM_004417 | 0.5 |
| BNO456 | KIF5B | kinesin family member 5B | Hs.149436 | NM_004521 | 6 |
| BNO457 | WTAP | Wilms' tumour 1-associating protein | Hs.119 | NM_004906 | 0.5 |
| BNO459 | FOS | v-fos FBJ murine osteosarcoma viral oncogene homolog | Hs.25647 | NM_005252 | 0.5 |
| BNO460 | GATA6 | GATA binding protein 6 | Hs.50924 | NM_005257 | 3 |
| BNO461 | HRY | hairy and enhancer of split 1, (Drosophila) | Hs.250666 | NM_005524 | 0.5 |
| BNO462 | SGK | serum/glucocorticoid regulated kinase | Hs.296323 | NM_005627 | 3 |
| BNO463 | TIEG | TGFB inducible early growth response | Hs.82173 | NM_005655 | 0.5 |
| BNO464 | BCAP31 | B-cell receptor-associated protein 31 | Hs.381232 | NM_005745 | |
| BNO465 | CALCRL | calcitonin receptor-like | Hs.152175 | NM_005795 | 24 |
| BNO466 | SUI1 | putative translation initiation factor | Hs.150580 | NM_005801 | 3 |
| BNO467 | TSC22 | transforming growth factor beta-stimulated protein TSC-22 | Hs.114360 | NM_006022 | 6 |
| BNO468 | RAN | RAN, member RAS oncogene family | Hs.426035 | NM_006325 | |
| BNO469 | LYPLA1 | lysophospholipase I | Hs.12540 | NM_006330 | 6 |
| BNO470 | SSFA2 | sperm specific antigen 2 | Hs.351355 | NM_006751 | 6 |
| BNO472 | CLIC4 | chloride intracellular channel 4 | Hs.25035 | NM_013943 | 24 |
| BNO473 | SLC7A11 | solute carrier family 7, member 11 | Hs.6682 | NM_014331 | 3 |
| BNO474 | RAI14 | retinoic acid induced 14 | Hs.15165 | NM_015577 | 6 |
| BNO475 | HSPC014 | chromosome 13 open reading frame 12 | Hs.279813 | NM_015932 | 24 |
| BNO476 | UMP- | UMP-CMP kinase | Hs.11463 | NM_016308 | 3 |
| | CMPK | | | | |
| BNO477 | SLC38A2 | solute carrier family 38, member 2 | Hs.298275 | NM_018976 | 3 |
| BNO478 | ZNF317 | zinc finger protein 317 | Hs.18587 | NM_020933 | 24 |
| BNO479 | RAB6C | RAB6C, member RAS oncogene family | Hs.333139 | NM_032144 | 24 |
| BNO480 | MKI67IP | MKI67 (FHA domain) interacting nucleolar phosphoprotein | Hs.142838 | NM_032390 | 3 |
| BNO481 | KPNA4 | karyopherin alpha 4 (importin alpha 3) | Hs.288193 | NM_002268 | 3 |
| BNO483 | C14orf32 | chromosome 14 open reading frame 32 | Hs.406401 | NM_144578 | 3 |
| BNO484 | SMARCA2 | SWI/SNF related, matrix associated, regulator of | Hs.198296 | NM_003070 | 0.5 |
| | | chromatin, A2 | | | |
| BNO485 | SOX4 | Homo sapiens SRY (sex determining region Y)-box 4 | Hs.83484 | NM_003107 | 3 |
| | | (SOX4), mRNA | | | |
| BNO487 | NR4A3 | nuclear receptor subfamily 4, group A, member 3 | Hs.80561 | NM_006981 | 0.5 |
| BNO488 | NTN4 | netrin 4 | Hs.102541 | NM_021229 | |
| BNO489 | DNCI2 | dynein, cytoplasmic, intermediate polypeptide 2 (DNCI2), | Hs.66881 | XM_027780 | 0.5 |
| | | mRNA | | | |
| BNO490 | UGCG | UDP-glucose ceramide glucosyltransferase | Hs.432605 | NM_003358 | 0.5, 24 |
| BNO491 | P125 | Sec23-interacting protein p125 | Hs.300208 | NM_007190 | 3 |
| BNO492 | NUDT4 | nudix (nucleoside diphosphate linked moiety X)-type motif 4 | Hs.355399 | NM_019094 | 6 |
| BNO495 | SATB1 | special AT-rich sequence binding protein 1 | Hs.74592 | NM_002971 | 6 |
| BNO496 | BZW1 | basic leucine zipper and W2 domains 1 | Hs.155291 | NM_014670 | 3 |
| BNO497 | TDG | thymine-DNA glycosylase | Hs.173824 | NM_003211 | 6 |
| BNO498 | ACTR3 | ARP3 actin-related protein 3 homolog (yeast) | Hs.380096 | NM_005721 | 24 |
| BNO499 | LAMP2 | lysosomal-associated membrane protein 2 | Hs.8262 | NM_013995 | 6 |
| BNO500 | ERBB2IP | erbb2 interacting protein | Hs.8117 | NM_018695 | 6 |
| BNO501 | DNAJB6 | DnaJ (Hsp40) homolog, subfamily B, member 6 | Hs.181195 | NM_005494 | 3 |
| BNO502 | EMP1 | epithelial membrane protein 1 | Hs.79368 | NM_001423 | 6 |
| BNO503 | MAPK1 | mitogen-activated protein kinase 1 | Hs.324473 | NM_002745 | 24 |
| BNO504 | CYP1A1 | cytochrome P450, subfamily 1, polypeptide 1 | Hs.72912 | NM_000499 | 6 |
| BNO505 | ACVR1 | activin A receptor, type I | Hs.150402 | NM_001105 | 3 |
| BNO506 | TPT1 | tumor protein, translationally-controlled 1 | Hs.401448 | NM_003295 | 0.5, 24 |
| BNO507 | VAV3 | vav 3 oncogene | Hs.267659 | NM_006113 | 3 |
| BNO508 | CAP | adenylyl cyclase-associated protein | Hs.104125 | NM_006367 | 24 |
| BNO509 | HSPA5 | Heat shock 70 kDa protein 5 (glucose-regulated protein, | Hs.75410 | NM_005347 | 6 |
| | | 78 kDa) | | | |
| BNO510 | TIA1 | TIA1 cytotoxic granule-associated RNA binding protein | Hs.239489 | NM_022173 | 6 |
| BNO511 | CCNT2 | cyclin T2 | Hs.155478 | NM_001241 | 6 |
| BNO512 | CHC1L | chromosome condensation 1-like | Hs.27007 | NM_001268 | 0.5 |
| BNO513 | SFPQ | splicing factor proline/glutamine rich | Hs.180610 | NM_005066 | 3 |
| BNO514 | PRKAR1A | protein kinase, cAMP-dependent, regulatory, type I, alpha | Hs.183037 | NM_002734 | 24 |
| BNO515 | RALA | v-ral simian leukemia viral oncogene homolog A (ras | Hs.6906 | NM_005402 | 6 |
| | | related) | | | |
| BNO516 | ANXA2 | annexin A2 | Hs.217493 | NM_004039 | 0.5 |
| BNO517 | NUP153 | nucleoporin 153 kDa | Hs.211608 | NM_005124 | 3 |
| BNO518 | RANBP9 | RAN binding protein 9 | Hs.279886 | NM_005493 | 24 |
| BNO519 | PRPF4B | PRP4 pre-mRNA processing factor 4 homolog B (yeast) | Hs.198891 | NM_003913 | 6 |
| BNO520 | TSN | translin | Hs.75066 | NM_004622 | 6 |
| BNO521 | H3F3A | H3 histone, family 3A | Hs.181307 | NM_002107 | 24 |
| BNO523 | PROS1 | protein S (alpha) | Hs.64016 | NM_000313 | 6 |
| BNO524 | DDX3 | DEAD/H (Asp-Glu-Ala-Asp/His) box polypeptide 3 | Hs.380774 | NM_001356 | 3 |
| BNO525 | TCF4 | transcription factor 4 | Hs.359289 | NM_003199 | 6 |
| BNO526 | PTP4A1 | Protein tyrosine phosphatase type IVA, member 1 | Hs.227777 | NM_003463 | 6 |
| BNO527 | BMPR2 | bone morphogenetic protein receptor, type II | Hs.53250 | NM_001204 | 3 |
| | | (serine/threonine kinase) | | | |
| BNO528 | NFE2L2 | nuclear factor (erythroid-derived 2)-like 2 | Hs.155396 | NM_006164 | 3 |
| BNO531 | AHR | aryl hydrocarbon receptor | Hs.170087 | NM_001621 | 3 |
| BNO532 | RANBP7 | RAN binding protein 7 | Hs.5151 | NM_006391 | 3 |
| BNO533 | ARF6 | ADP-ribosylation factor 6 | Hs.89474 | NM_001663 | 3 |
| BNO534 | SCARF1 | SCARF1 Scavenger receptor class F, member 1 | Hs.57735 | NM_003693E | 24 |
| BNO535 | PLU-1 | putative DNA/chromatin binding motif | Hs.143323 | NM_006618 | 24 |
| BNO536 | TOMM20 | translocase of outer mitochondrial membrane 20 (yeast) | Hs.75187 | NM_014765 | 6 |
| | | homolog | | | |
| BNO537 | B2M | beta-2-microglobulin | Hs.48516 | NM_004048 | 24 |
| BNO538 | zizimin1 | zizimin1 | Hs.8021 | NM_015296 | 6 |
| BNO539 | ARPP-19 | cyclic AMP phosphoprotein, 19 kD | Hs.7351 | NM_006628 | 3 |
| BNO540 | RAP1B | RAP1B, member of RAS oncogene family | Hs.156764 | NM_015646 | 3 |
| BNO541 | MCP | membrane cofactor protein | Hs.83532 | NM_153826 | 6 |
| BNO542 | IFI16 | interferon, gamma-inducible protein 16 | Hs.155530 | NM_005531 | 0.5 |
| BNO543 | PRG1 | proteoglycan 1, secretory granule | Hs.1908 | NM_002727 | |
| BNO544 | KIT | v-kit Hardy-Zuckerman 4 feline sarcoma viral oncogene | Hs.81665 | NM_000222 | 0.5, 24 |
| | | homolog | | | |
| BNO545 | SYBL1 | synaptobrevin-like 1 | Hs.24167 | NM_005638 | 6 |
| BNO546 | TCF8 | transcription factor 8 (represses interleukin 2 expression) | Hs.232068 | NM_030751E | 6 |
| BNO548 | NXF1 | nuclear RNA export factor 1 | Hs.323502 | NM_006362 | 3, 24 |
| BNO549 | RAP2B | RAP2B, member of RAS oncogene family | Hs.239527 | NM_002886 | 3 |
| BNO551 | IL6ST | interleukin 6 signal transducer (gp130, oncostatin M | Hs.82065 | NM_002184 | 6 |
| | | receptor) | | | |
| BNO552 | REST | RE1-silencing transcription factor | Hs.401145 | NM_005612 | 6 |
| BNO553 | SLC19A2 | solute carrier family 19 (thiamine transporter), member 2 | Hs.30246 | NM_006996 | 3 |
| BNO554 | EIF4G2 | eukaryotic translation initiation factor 4 gamma, 2 | Hs.183684 | NM_001418 | 3 |
| BNO555 | PTPRE | protein tyrosine phosphatase, receptor type, E | Hs.31137 | NM_006504 | 3 |
| BNO556 | PDE3A | phosphodiesterase 3A, cGMP-inhibited | Hs.777 | NM_000921 | 3 |
| BNO557 | C1QR1 | complement component 1, q subcomponent, receptor 1 | Hs.97199 | NM_012072 | 24 |
| BNO558 | RANBP2 | RAN binding protein 2 | Hs.199179 | NM_006267 | |
| BNO559 | KIS | kinase interacting with leukemia-associated gene (stathmin) | Hs.127310 | NM_144624 | 24 |
| BNO560 | HMGCR | 3-hydroxy-3-methylglutaryl-Coenzyme A reductase | Hs.11899 | NM_000859 | 6 |
| BNO561 | PDCD4 | programmed cell death 4 (neoplastic transformation | Hs.326248 | NM_145341 | 3 |
| | | inhibitor) | | | |
| BNO562 | TACC1 | transforming, acidic coiled-coil containing protein 1 | Hs.173159 | NM_006283 | 0.5 |
| BNO564 | DIS3 | mitotic control protein dis3 homolog | Hs.323346 | NM_014953 | 6 |
| BNO565 | TOP2A | topoisomerase (DNA) II alpha 170 kDa | Hs.156346 | NM_001067 | 6 |
| BNO566 | SLC7A2 | solute carrier family 7, member 2 | Hs.153985 | NM_003046 | 6 |
| BNO567 | FH | fumarate hydratase | Hs.75653 | NM_000143 | 6 |
| BNO568 | IL1RL1 | interleukin 1 receptor-like 1 | Hs.66 | NM_003856 | 6 |
| BNO569 | HPRP3P | U4/U6-associated RNA splicing factor | Hs.11776 | NM_004698 | 6 |
| BNO570 | DDX5 | DEAD/H (Asp-Glu-Ala-Asp/His) box polypeptide 5 | Hs.76053 | NM_004396 | |
| BNO571 | MAD2L1 | MAD2 mitotic arrest deficient-like 1 (yeast) | Hs.79078 | NM_002358 | 0.5, 24 |
| BNO572 | MADH7 | MAD, mothers against decapentaplegic homolog 7 | Hs.100602 | NM_005904 | 3 |
| | | (Drosophila) | | | |
| BNO573 | E2F3 | E2F transcription factor 3 | Hs.1189 | NM_001949 | 3 |
| BNO574 | CSNK2A2 | CSNK2A2 Casein kinase 2, alpha prime polypeptide | Hs.82201 | NM_001896 | 6 |
| BNO575 | MAX | MAX protein | Hs.42712 | NM_002382 | 6 |
| BNO576 | ERAP140 | 140 kDa estrogen receptor associated protein | Hs.339283 | AF493978 | 3 |
| BNO577 | CD9 | CD9 antigen (p24) | Hs.1244 | NM_001769 | 24 |
| BNO578 | ATRX | alpha thalassemia/mental retardation syndrome X-linked | Hs.96264 | NM_000489 | 6 |
| BNO579 | YWHAZ | tyrosine/tryptophan activation protein, zeta polypeptide | Hs.75103 | NM_003406 | 3 |
| BNO580 | IDS | iduronate 2-sulfatase (Hunter syndrome) | Hs.172458 | NM_000202 | 24 |
| BNO581 | SERPINE2 | serine (or cysteine) proteinase inhibitor, clade E, member 2 | Hs.21858 | NM_006216 | 6 |
| BNO582 | DDEF1 | development and differentiation enhancing factor 1 | Hs.10669 | NM_018482 | 6 |
| BNO583 | GLRX | glutaredoxin (thioltransferase) | Hs.28988 | NM_002064 | 24 |
| BNO584 | MAP3K1 | MAP3K1 Mitogen-activated protein kinase kinase kinase 1 | Hs.170610 | XM_042066 | 3 |
| BNO585 | ANKH | ankylosis, progressive homolog (mouse) | Hs.168640 | NM_054027 | 3 |
| BNO586 | RBX1 | ring-box 1 | Hs.279919 | NM_014248 | 24 |
| BNO587 | NAB1 | NGFI-A binding protein 1 (EGR1 binding protein 1) | Hs.107474 | NM_005966 | 3 |
| BNO588 | TNFSF10 | tumor necrosis factor (ligand) superfamily, member 10 | Hs.83429 | NM_003810 | 3 |
| BNO589 | PRDX3 | peroxiredoxin 3 | Hs.75454 | NM_006793 | 6 |
| BNO590 | MAP2K1 | mitogen-activated protein kinase kinase 1 | Hs.3446 | NM_002755 | 3 |
| BNO591 | NFATC1 | nuclear factor of activated T-cells, calcineurin-dependent 1 | Hs.96149 | NM_006162 | 24 |
| BNO594 | USP7 | ubiquitin specific protease 7 (herpes virus-associated) | Hs.78683 | NM_003470 | |
| BNO595 | ARHB | ras homolog gene family, member B | Hs.406064 | NM_004040 | 3 |
| BNO596 | PTEN | phosphatase and tensin homolog | Hs.10712 | NM_000314 | |
| BNO597 | UBL1 | ubiquitin-like 1 (sentrin) | Hs.81424 | NM_003352 | 24 |
| BNO598 | RAB5A | RAB5A, member RAS oncogene family | Hs.73957 | NM_004162 | 3 |
| BNO599 | ITGB1 | integrin, beta 1 | Hs.287797 | NM_002211 | 24 |
| BNO600 | PRDM2 | PR domain containing 2, with ZNF domain | Hs.26719 | NM_012231 | 6 |
| BNO602 | ITGA2 | integrin, alpha 2 (CD49B, alpha 2 subunit of VLA-2 | Hs.271986 | NM_002203 | 6 |
| | | receptor) | | | |
| BNO603 | ETV5 | ets variant gene 5 (ets-related molecule) | Hs.43697 | NM_004454 | 3 |
| BNO604 | ZFHX1B | zinc finger homeobox 1b | Hs.34871 | NM_014795 | 3 |
| BNO606 | LOC157713 | lysophospholipase I-like pseudogene on chromosome 6 | None | NG_001063 | |
| BNO607 | RBM3 | RNA binding motif protein 3 | Hs.301404 | NM_006743 | 0.5 |
| BNO609 | NET-6 | transmembrane 4 superfamily member tetraspan NET-6 | Hs.364544 | NM_014399 | 6 |
| BNO610 | EHD3 | EH-domain containing 3 | Hs.87125 | NM_014600 | 24 |
| BNO611 | KIAA0992 | palladin | Hs.194431 | NM_016081 | 6 |
| BNO613 | METL | methyltransferase like 2 | Hs.433213 | NM_018396 | 3 |
| BNO614 | HT010 | uncharacterized hypothalamus protein HT010 | Hs.6375 | NM_018471 | 0.5 |
| BNO615 | C3orf4 | chromosome 3 open reading frame 4 | Hs.107393 | NM_019895 | 6 |
| BNO619 | RPL27A | ribosomal protein L27a | Hs.76064 | NM_000990 | 6 |
| BNO621 | MIB | Ubiquitin ligase mind bomb | Hs.34892 | AY149908 | 0.5 |
| BNO623 | KIAA0261 | KIAA0261 protein | Hs.154978 | XM_042946 | 24 |
| BNO624 | KIAA1199 | KIAA1199 protein | Hs.50081 | XM_051860 | 6 |
| BNO625 | HIF1 | huntingtin interacting protein B | Hs.6947 | NM_014159 | |
| BNO642 | ETL | EGF-TM7-latrophilin-related protein | Hs.57958 | NM_022159 | 24 |
| BNO643 | VMP1 | likely ortholog of rat vacuole membrane protein 1 | Hs.166254 | NM_030938 | 3 |
| BNO644 | TAF9 | TATA box binding protein (TBP)-associated factor, 32 kDa | Hs.60679 | NM_016283 | 24 |
| BNO646 | MAN1A1 | mannosidase, alpha, class 1A, member 1 | Hs.432931 | NM_005907 | 6 |
| BNO647 | DOCK4 | Dedicator of cytokinesis 4 | Hs.118140 | NM_014705 | 24 |
| BNO649 | ADAMTS9 | a disintegrin-like and metalloprotease (thrombospondin type | Hs.126855 | NM_020249 | 24 |
| | | 1 motif, 9) | | | |
| BNO650 | CSNK2A2 | Casein kinase 2, alpha prime polypeptide | Hs.82201 | NM_001896 | 6 |
| BNO651 | RPLP0 | ribosomal protein, large, P0 | Hs.406511 | NM_001002 | 6 |
| BNO653 | GALNT4 | N-acetylgalactosaminyltransferase 4 | Hs.271923 | NM_003774 | 3 |
| BNO654 | GNG2 | guanine nucleotide binding protein (G protein), gamma 2 | Hs.289026 | BC020774 | 6 |
| BNO656 | MBNL | muscleblind-like (Drosophila) | Hs.28578 | NM_021038 | |
| BNO657 | ARL8 | ADP-ribosylation factor-like 8 | Hs.25362 | BC024163 | 3 |
| BNO658 | ASB3 | ankyrin repeat and SOCS box-containing 3 | Hs.9893 | NM_016115 | 6 |
| BNO660 | GG2-1 | TNF-induced protein | Hs.17839 | NM_014350 | 3 |
| BNO661 | ELL2 | ELL-related RNA polymerase II, elongation factor | Hs.98124 | NM_012081 | 3 |
| BNO663 | ATP5J2 | ATP synthase, H+ transporting, mitochondrial F0 complex, | Hs.235557 | NM_004889 | 24 |
| | | subunit f 2 | | | |
| BNO665 | SDCBP | syndecan binding protein (syntenin) | Hs.8180 | NM_005625 | 3 |
| BNO666 | KIAA1959 | Nm23-phosphorylated unknown substrate | Hs.55067 | NM_032873 | 3 |
| BNO667 | GNPNAT1 | glucosamine-phosphate N-acetyltransferase 1 | Hs.478025 | NM_198066 | 6 |
| BNO668 | SPRED1 | Sprouty-related, EVH1 domain containing 1 | Hs.132804 | NM_152594 | 3, 24 |
| BNO670 | Nbak2 | homeodomain interacting protein kinase 1-like protein | Hs.12259 | NM_152696 | 6 |
| BNO672 | GABPA | GA binding protein transcription factor, alpha subunit 60 kDa | Hs.78 | NM_002040 | 3 |
| BNO674 | V-1 | likely ortholog of rat V-1 protein | Hs.21321 | NM_145808 | 24 |
| BNO676 | C8FW | phosphoprotein regulated by mitogenic pathways | Hs.7837 | NM_025195 | 3 |
| BNO678 | TBC1D4 | TBC1 domain family, member 4 | Hs.173802 | NM_014832 | 6 |
| BNO679 | ACATE2 | likely ortholog of mouse acyl-Coenzyme A thioesterase 2 | Hs.18625 | NM_012332 | 24 |
| BNO680 | CRYZ | crystallin, zeta (quinone reductase) | Hs.83114 | NM_001889 | 6 |
| BNO681 | KPNB1 | karyopherin (importin) beta 1 | Hs.180446 | NM_002265 | 24 |
| BNO682 | RPL23A | ribosomal protein L23a | Hs.350046 | NM_000984 | 0.5 |
| BNO683 | LIMS1 | LIM and senescent cell antigen-like domains 1 | Hs.112378 | NM_004987 | 6 |
| BNO684 | WW45 | WW45 protein | Hs.288906 | NM_021818 | 3 |
| BNO686 | ST3GALVI | alpha2,3-sialyltransferase | Hs.34578 | NM_006100 | 6 |
| BNO688 | CPR8 | cell cycle progression 8 protein | Hs.283753 | NM_004748 | 24 |
| BNO689 | HDCL | hHDC for homolog of Drosophila headcase | Hs.6679 | NM_016217 | 3 |
| BNO691 | UBC | ubiquitin C | Hs.183704 | NM_021009 | 3 |
| BNO692 | RDX | radixin | Hs.263671 | NM_002906 | 24 |
| BNO693 | PELI1 | pellino homolog 1 (Drosophila) | Hs.7886 | NM_020651 | 3 |
| BNO695 | MCC | mutated in colorectal cancers | Hs.1345 | NM_002387 | 6 |
| BNO696 | RetSDR2 | RetSDR2 Retinal short-chain dehydrogenase/reductase 2 | Hs.282984 | NM_016245 | 3 |
| BNO698 | CSS3 | Chondroitin sulfate synthase 3 | Hs.165050 | AB086062 | 3 |
| BNO699 | BRE | brain and reproductive organ-expressed (TNFRSF1A | Hs.80426 | NM_004899 | 6 |
| | | modulator) | | | |
| BNO701 | BAZ1A | bromodomain adjacent to zinc finger domain, 1A | Hs.8858 | NM_013448 | 3 |
| BNO702 | HNRPDL | heterogeneous nuclear ribonucleoprotein D-like | Hs.372673 | NM_005463 | 3 |
| BNO703 | PREI3 | preimplantation protein 3 | Hs.107942 | NM_015387 | 6 |
| BNO707 | BNO707 | Human XIST, coding sequence “a” | Hs.83623 | X56199 | 3 |
| BNO709 | ROD1 | ROD1 regulator of differentiation 1 (S. pombe) | Hs.374634 | NM_005156 | 6 |
| BNO711 | SMAP-5 | golgi membrane protein SB140 | Hs.5672 | NM_030799 | 6 |
| BNO715 | M-RIP | Myosin phosphatase-Rho interacting protein | Hs.430725 | AB020671 | 0.5, 24 |
| BNO716 | HIVEP2 | human immunodeficiency virus type I enhancer binding | Hs.75063 | NM_006734 | 3 |
| | | protein 2 | | | |
| BNO717 | DC42 | hypothetical protein DC42 | None | NM_030921 | 3 |
| BNO718 | GRPEL2 | GrpE-like 2, mitochondrial | Hs.17121 | NM_152407 | 6 |
| BNO719 | PCMF | potassium channel modulatory factor | Hs.5392 | NM_020122 | 3 |
| BNO720 | UBE2E1 | ubiquitin-conjugating enzyme E2E 1 (UBC4/5 homolog, | Hs.163546 | NM_003341 | 24 |
| | | yeast) | | | |
| BNO721 | KLHL4 | kelch-like 4 (Drosophila) | Hs.49075 | NM_019117 | |
| BNO722 | MANEA | Mannosidase, endo-alpha | Hs.46903 | NM_024641 | 3 |
| BNO724 | TCF12 | transcription factor 12 (HTF4, helix-loop-helix transcription | Hs.21704 | NM_003205 | 6 |
| | | factors 4) | | | |
| BNO726 | STAF42 | SPT3-associated factor 42 | Hs.435967 | NM_053053 | 6 |
| BNO727 | CYFIP1 | cytoplasmic FMR1 interacting protein 1 | Hs.77257 | NM_014608 | 6 |
| BNO728 | NOL5A | nucleolar protein 5A (56 kDa with KKE/D repeat) | Hs.376064 | NM_006392 | 6 |
| BNO729 | GSA7 | ubiquitin activating enzyme E1-like protein | Hs.278607 | NM_006395 | 6 |
| BNO732 | P66 Alpha | P66 Alpha | Hs.118964 | NM_017660 | |
| BNO733 | STAG1 | stromal antigen 1 | Hs.286148 | NM_005862 | |
| BNO734 | MYCT1 | Myc target 1 | Hs.18160 | NM_025107 | 6 |
| BNO736 | SCAMP1 | secretory carrier membrane protein 1 | Hs.31218 | NM_004866 | 3 |
| BNO738 | ACTG1 | actin, gamma 1 | Hs.14376 | NM_001614 | 0.5 |
| BNO739 | HRB2 | HIV-1 rev binding protein 2 | Hs.154762 | NM_007043 | 6 |
| BNO741 | VMP1 | Likely orthologue of rat vacuole membrane protein 1 | Hs.166254 | NM_030938 | 6 |
| BNO743 | BCAT1 | branched chain aminotransferase 1, cytosolic | Hs.438993 | NM_005504 | 0.5, 24 |
| BNO744 | PJA2 | Praja 2, RING-H2 motif containing | Hs.224262 | NM_014819 | |
| BNO746 | FKSG14 | leucine zipper protein FKSG14 | Hs.192843 | NM_022145 | 6 |
| BNO748 | KLHL6 | kelch-like 6 (Drosophila) | Hs.43616 | NM_130446 | 6 |
| BNO749 | TTL | Tubulin tyrosine ligase | Hs.358997 | NM_153712 | 6 |
| BNO750 | CDC23 | CDC23 (cell division cycle 23, yeast, homolog) | Hs.153546 | NM_004661 | 24 |
| BNO751 | ULK2 | unc-51-like kinase 2 (C. elegans) | Hs.151406 | NM_014683 | 3 |
| BNO752 | SCARB2 | SCARB2 Scavenger receptor class B, member 2 | Hs.323567 | NM_005506E | 3 |
| BNO755 | ZMPSTE24 | zinc metalloproteinase (STE24 homolog, yeast) | Hs.25846 | NM_005857 | |
| BNO757 | U5-100K | prp28, U5 snRNP 100 kd protein | Hs.184771 | NM_004818 | |
| BNO758 | CHD4 | chromodomain helicase DNA binding protein 4 | Hs.74441 | NM_001273 | 6 |
| BNO760 | CGI-127 | yippee protein | Hs.184542 | NM_016061 | 3, 24 |
| BNO763 | BET1 | BET1 homolog (S. cerevisiae) | Hs.23103 | NM_005868 | 24 |
| BNO764 | ARHGAP5 | Rho GTPase activating protein 5 | Hs.267831 | NM_001173 | |
| BNO765 | TUBA | Scaffold protein TUBA | Hs.429994 | NM_015221 | 3 |
| BNO766 | NUMB | numb homolog (Drosophila) | Hs.78890 | NM_003744 | 6 |
| BNO767 | P5 | protein disulfide isomerase-related protein | Hs.182429 | NM_005742 | 0.5 |
| BNO769 | SFRS2IP | splicing factor, arginine/serine-rich 2, interacting protein | Hs.51957 | NM_004719 | 6 |
| BNO770 | OXA1L | oxidase (cytochrome c) assembly 1-like | Hs.151134 | NM_005015 | 0.5, 24 |
| BNO771 | POH1 | 26S proteasome-associated pad1 homolog | Hs.178761 | NM_005805 | 6 |
| BNO773 | AHCYL1 | S-adenosylhomocysteine hydrolase-like 1 | Hs.4113 | NM_006621 | 3 |
| BNO774 | UAP1 | UDP-N-acteylglucosamine pyrophosphorylase 1 | Hs.21293 | NM_003115 | 3 |
| BNO775 | PLS3 | plastin 3 (T isoform) | Hs.4114 | NM_005032 | 6 |
| BNO776 | TSNAX | translin-associated factor X | Hs.96247 | NM_005999 | 0.5 |
| BNO777 | HELO1 | homolog of yeast long chain polyunsaturated fatty acid | Hs.250175 | NM_021814 | 6 |
| | | elong. enz. 2 | | | |
| BNO778 | MAN2A1 | mannosidase, alpha, class 2A, member 1 | Hs.377915 | NM_002372 | 3 |
| BNO779 | RAB21 | RAB21, member RAS oncogene family | Hs.184627 | NM_014999 | 6 |
| BNO781 | WAC | WW domain-containing adapter with a coiled-coil region | Hs.70333 | NM_016628 | 3 |
| BNO783 | POSH | likely ortholog of mouse plenty of SH3 domains | Hs.301804 | AB040927 | 6 |
| BNO784 | RBM9 | RNA binding motif protein 9 | Hs.433574 | NM_014309 | |
| BNO785 | CSRP2 | cysteine and glycine-rich protein 2 | Hs.10526 | NM_001321 | 3 |
| BNO786 | COPA | coatomer protein complex, subunit alpha | Hs.75887 | NM_004371 | 6 |
| BNO787 | TIMM17A | translocase of inner mitochondrial membrane 17 homolog A | Hs.20716 | NM_006335 | 6 |
| | | (yeast) | | | |
| BNO788 | RIN2 | Ras and Rab interactor 2 | Hs.62349 | NM_018993 | 24 |
| BNO789 | KLHL5 | kelch-like 5 (Drosophila) | Hs.272239 | NM_015990 | 24 |
| BNO790 | IPLA2(γ) | intracellular memb.-assoc. calcium-independent | Hs.44198 | AF263613 | 6 |
| | | phospholipase A2γ | | | |
| BNO794 | SMARCA5 | SWI/SNF related regulator of chromatin, a5 | Hs.9456 | NM_003601 | |
| BNO796 | FBXL3A | F-box and leucine-rich repeat protein 3A | Hs.7540 | NM_012158 | 24 |
| BNO797 | SART2 | squamous cell carcinoma antigen recognized by T cell | Hs.58636 | NM_013352E | 6 |
| BNO798 | YWHAZ | 14-3-3zeta | Hs.386834 | NM_145690 | |
| BNO799 | SH3BGRL2 | SH3 domain binding glutamic acid-rich protein like 2 | Hs.9167 | NM_031469 | 3, 24 |
| BNO801 | PUM1 | pumilio homolog 1 (Drosophila) | Hs.153834 | NM_014676 | 3 |
| BNO803 | CCT2 | chaperonin containing TCP1, subunit 2 (beta) | Hs.432970 | NM_006431 | 6 |
| BNO804 | PTPRK | protein tyrosine phosphatase, receptor type, K | Hs.79005 | NM_002844 | 6 |
| BNO806 | TM4SF1 | transmembrane 4 superfamily member 1 | Hs.351316 | NM_014220 | 6 |
| BNO807 | CHSY1 | carbohydrate (chondroitin) synthase 1 | Hs.110488 | NM_014918 | 24 |
| BNO808 | TERF2IP | telomeric repeat binding factor 2, interacting protein | Hs.274428 | NM_018975 | 6 |
| BNO809 | RDC1 | G protein-coupled receptor | Hs.23016 | BC036661 | 3 |
| BNO810 | CD59 | CD59 antigen p18-20 | Hs.278573 | AK095453 | 0.5, 6 |
| BNO811 | UBE2D1 | ubiquitin-conjugating enzyme E2D 1 (UBC4/5 homolog, | Hs.129683 | NM_003338 | 6 |
| | | yeast) | | | |
| BNO813 | CUL4B | cullin 4B | Hs.155976 | NM_003588 | 24 |
| BNO814 | LCHN | LCHN protein | Hs.233044 | AB032973 | 3 |
| BNO815 | PELO | pelota homolog (Drosophila) | Hs.5798 | NM_015946 | 3 |
| BNO817 | MRPS10 | mitochondrial ribosomal protein S10 | Hs.380887 | NM_018141 | 6 |
| BNO820 | EIF3S2 | eukaryotic translation initiation factor 3, subunit 2 beta, | Hs.192023 | NM_003757 | 3 |
| | | 36 kDa | | | |
| BNO822 | UBQLN1 | ubiquilin 1 | Hs.9589 | NM_013438 | 3 |
| BNO823 | PSMB3 | proteasome (prosome, macropain) subunit, beta type, 3 | Hs.82793 | NM_002795 | 0.5, 24 |
| BNO826 | UBE2J1 | ubiquitin-conjugating enzyme E2, J1 (UBC6 homolog, | Hs.184325 | NM_016336 | 24 |
| | | yeast) | | | |
| BNO827 | CDK2AP1 | CDK2-associated protein 1 | Hs.433201 | NM_004642 | 24 |
| BNO828 | CRY1 | cryptochrome 1 (photolyase-like) | Hs.151573 | NM_004075 | 3 |
| BNO830 | HSPC051 | ubiquinol-cytochrome c reductase complex (7.2 kD) | Hs.284292 | NM_013387 | 6 |
| BNO832 | GNG11 | guanine nucleotide binding protein (G protein), gamma 11 | Hs.83381 | NM_004126 | 0.5, 24 |
| BNO834 | ZNF198 | zinc finger protein 198 | Hs.109526 | NM_003453 | 6 |
| BNO835 | RAB11A | RAB11A, member RAS oncogene family | Hs.75618 | NM_004663 | 6 |
| BNO836 | SMAP1 | stromal membrane-associated protein | Hs.373517 | NM_021940 | 6 |
| BNO837 | COPG | Coatomer protein complex, subunit gamma | Hs.368056 | NM_016128 | 3 |
| BNO839 | MTHFD2 | methylene tetrahydrofolate dehydrogenase (NAD+ | Hs.154672 | NM_006636 | 3 |
| | | dependent) | | | |
| BNO840 | PODXL | podocalyxin-like | Hs.16426 | NM_005397 | 6 |
| BNO841 | SLC30A7 | Solute carrier family 30 (zinc transporter), member 7 | Hs.38856 | NM_133496 | 3 |
| BNO842 | API5 | apoptosis inhibitor 5 | Hs.227913 | NM_006595 | 3 |
| BNO843 | ERdj5 | ER-resident protein ERdj5 | Hs.1098 | NM_018981 | 3 |
| BNO844 | HDGFRP3 | Hepatoma-derived growth factor, related protein 3 | Hs.127842 | NM_016073 | 6 |
| BNO847 | TUCAN | tumor up-regulated CARD-containing antagonist of caspase | Hs.10031 | NM_014959 | 6 |
| | | nine | | | |
| BNO850 | PCDH17 | protocadherin 17 | Hs.106511 | NM_014459 | 24 |
| BNO851 | GALNT10 | N-acetylgalactosaminyltransferase 10 | Hs.107260 | NM_017540 | 24 |
| BNO853 | UQCRC1 | ubiquinol-cytochrome c reductase core protein I | Hs.119251 | NM_003365 | 6 |
| BNO854 | RPL3 | ribosomal protein L3 | Hs.119598 | NM_000967 | 24 |
| BNO855 | CMT2 | gene predicted from cDNA with a complete coding | Hs.124 | NM_014628 | 24 |
| | | sequence | | | |
| BNO858 | PSMD7 | proteasome 26S subunit, non-ATPase, 7 | Hs.155543 | NM_002811 | 6 |
| BNO859 | CCT5 | chaperonin containing TCP1, subunit 5 (epsilon) | Hs.1600 | NM_012073 | 3 |
| BNO860 | SEC5 | homolog of yeast Sec5 | Hs.16580 | NM_018303 | 6 |
| BNO861 | SKP1A | S-phase kinase-associated protein 1A (p19A) | Hs.171626 | NM_006930 | 24 |
| BNO863 | CAPZA1 | capping protein (actin filament) muscle Z-line, alpha 1 | Hs.184270 | NM_006135 | 24 |
| BNO864 | YES1 | v-yes-1 Yamaguchi sarcoma viral oncogene homolog 1 | Hs.194148 | NM_005433 | 24 |
| BNO865 | DAAM1 | dishevelled associated activator of morphogenesis 1 | Hs.197751 | NM_014992 | 6 |
| BNO866 | BCL6B | B-cell CLL/lymphoma 6, member B (zinc finger protein) | Hs.22575 | NM_181844 | 6 |
| BNO872 | AF5Q31 | ALL1 fused gene from 5q31 | Hs.231967 | NM_014423 | 6 |
| BNO874 | ALDH9A1 | aldehyde dehydrogenase 9 family, member A1 | Hs.2533 | NM_000696 | 24 |
| BNO875 | CDC42EP3 | CDC42 effector protein (Rho GTPase binding) 3 | Hs.260024 | NM_006449 | 0.5, 24 |
| BNO877 | MIS12 | homolog of yeast Mis12 | Hs.267194 | NM_024039 | 6 |
| BNO879 | ATP6V1D | ATPase, H+ transporting, lysosomal 34 kDa, V1 subunit D | Hs.272630 | NM_015994 | 6 |
| BNO880 | VCIP135 | valosin-containing protein (p97)/p47 complex-interacting | Hs.287727 | NM_025054 | 6 |
| | | protein p135 | | | |
| BNO882 | D10S170 | DNA segment on chromosome 10 (unique) 170 | Hs.288862 | NM_005436 | 6 |
| BNO884 | ARPC3 | actin related protein 2/3 complex, subunit 3, 21 kDa | Hs.293750 | NM_005719 | 24 |
| BNO885 | RPS19 | ribosomal protein S19 | Hs.298262 | NM_001022 | 6 |
| BNO888 | NEUGRIN | mesenchymal stem cell protein DSC92 | Hs.323467 | NM_016645 | 6 |
| BNO889 | CALD1 | caldesmon 1 | Hs.325474 | NM_033138 | 0.5 |
| BNO891 | NFIB | nuclear factor I/B | Hs.33287 | NM_005596 | 0.5 |
| BNO893 | HSPCA | heat shock 90 kDa protein 1, alpha | Hs.356531 | NM_005348 | 6 |
| BNO896 | NSAP1 | NS1-associated protein 1 | Hs.373499 | NM_006372 | 6 |
| BNO897 | SYT11 | synaptotagmin XI | Hs.380439 | NM_152280 | 6 |
| BNO899 | HNRPC | heterogeneous nuclear ribonucleoprotein C (C1/C2) | Hs.406125 | NM_006321 | 24 |
| BNO900 | STMN1 | stathmin 1/oncoprotein 18 | Hs.406269 | NM_005563 | 6 |
| BNO901 | ATP5B | ATP synthase, H+ transporting, mitochondrial F1 complex, | Hs.406510 | NM_001686 | 0.5, 24 |
| | | beta | | | |
| BNO902 | PSMB1 | proteasome (prosome, macropain) subunit, beta type, 1 | Hs.407981 | NM_002793 | 0.5, 24 |
| BNO903 | DDX10 | DEAD/H (Asp-Glu-Ala-Asp/His) box polypeptide 10 (RNA | Hs.41706 | NM_004398 | 6 |
| | | helicase) | | | |
| BNO904 | RPL36AL | ribosomal protein L36a-like | Hs.419465 | NM_001001 | 24 |
| BNO907 | NDUFV2 | NADH dehydrogenase (ubiquinone) flavoprotein 2, 24 kDa | Hs.51299 | NM_021074 | 0.5, 24 |
| BNO909 | DCK | deoxycytidine kinase | Hs.709 | NM_000788 | 24 |
| BNO911 | MDH1 | malate dehydrogenase 1, NAD (soluble) | Hs.75375 | NM_005917 | 24 |
| BNO912 | SERP1 | stress-associated endoplasmic reticulum protein 1 | Hs.76698 | NM_014445 | 0.5 |
| BNO913 | RPS3A | ribosomal protein S3A | Hs.77039 | NM_001006 | 0.5 |
| BNO914 | ARHA | ras homolog gene family, member A | Hs.77273 | NM_001664 | 0.5 |
| BNO915 | LAMA4 | laminin, alpha 4 | Hs.78672 | NM_002290 | 6 |
| BNO916 | SNX9 | sorting nexin 9 | Hs.7905 | NM_016224 | 6 |
| BNO918 | RAD21 | RAD21 homolog (S. pombe) | Hs.81848 | NM_006265 | 0.5, 24 |
| BNO920 | PHLDA1 | pleckstrin homology-like domain, family A, member 1 | Hs.82101 | NM_007350 | 6 |
| BNO921 | ARHGDIB | Rho GDP dissociation inhibitor (GDI) beta | Hs.83656 | NM_001175 | 24 |
| BNO922 | ELP2 | elongator protein 2 | Hs.8739 | NM_018255 | 6 |
| BNO924 | ATP6V1G1 | ATPase, H+ transporting, lysosomal 13 kDa, V1 subunit G | Hs.90336 | NM_004888 | 24 |
| | | isoform 1 | | | |
| BNO925 | DNAJA1 | DnaJ (Hsp40) homolog, subfamily A, member 1 | Hs.94 | NM_001539 | 3 |
| BNO927 | CYB561 | cytochrome b-561 | None | NM_001915 | 24 |
| BNO947 | HNRPDL | Heterogeneous nuclear ribonucleoprotein D-like | Hs.372673 | NM_005463 | 3 |
| BNO952 | ARHB | Ras homolog gene family, member B | Hs.406064 | NM_004040 | 3 |
| BNO955 | CYB561 | Cytochrome b-561 | Hs.355264 | AK095244 | 24 |
| BNO958 | ATP6 | ATP synthase F0 subunit 6 - mitochondrial gene | None | NC_001807 | 24 |
| BNO969 | ND4L | NADH dehydrogenase subunit 4L - mitochondrial gene | None | NC_001807 | 6 |
| BNO960 | COX2 | cytochrome C oxidase subunit II - mitochondrial gene | None | NC_001807 | 0.5, 24 |
| BNO1014 | SET | SET translocation (myeloid leukemia-associated) | Hs.145279 | NM_003011 | 6 |
| BNO1015 | JUNB | jun B proto-oncogene | Hs.400124 | NM_002229 | 0.5 |
| BNO1016 | HMGB1 | high-mobility group box 1 | Hs.6727 | NM_002128 | 6 |
| BNO1017 | PAFAH1B2 | Platelet-activating factor acetylhydrolase, isoform Ib, beta | Hs.93354 | NM_002572 | 24 |
| | | subunit | | | |

TABLE 3
Genes Previously Associated with Angiogenesis

| | | | | | Peak |
|---|---|---|---|---|---|
| BNO | | | UniGene | | Expression |
| Number | Symbol | Gene Description - Homology | Number | GenBank Number | (h) |
| BNO435 | ICAM1 | intercellular adhesion molecule 1 (CD54), human rhinovirus | Hs.168383 | NM_000201 | 3 |
| | | receptor | | | |
| BNO437 | IL8 | interleukin 8 | Hs.624 | NM_000584 | 3 |
| BNO439 | VCAM1 | vascular cell adhesion molecule 1 | Hs.109225 | NM_001078 | 3 |
| BNO440 | ANGPT2 | angiopoietin 2 | Hs.115181 | NM_001147 | 6 |
| BNO444 | CTNNB1 | catenin (cadherin-associated protein), beta 1, 88 kDa | Hs.171271 | NM_001904 | 3 |
| BNO445 | F3 | coagulation factor III (thromboplastin, tissue factor) | Hs.62192 | NM_001993 | 3 |
| BNO450 | STC1 | stanniocalcin 1 | Hs.25590 | NM_003155 | 24 |
| BNO458 | ADAMTS4 | a disintegrin-like and metalloprotease (thrombospondin type | Hs.211604 | NM_005099 | 6 |
| | | 1 motif, 4) | | | |
| BNO471 | ESM1 | endothelial cell-specific molecule 1 | Hs.41716 | NM_007036 | 3, 24 |
| BNO482 | CMG2 | capillary morphogenesis protein 2 | Hs.5897 | NM_058172 | 6 |
| BNO486 | EFNB2 | ephrin-B2 | Hs.30942 | NM_004093 | 3 |
| BNO493 | PTGS1 | prostaglandin-endoperoxide synthase 1 | Hs.88474 | NM_000962 | 6 |
| BNO494 | KDR | kinase insert domain receptor (a type III receptor tyrosine | Hs.12337 | NM_002253 | |
| | | kinase) | | | |
| BNO522 | F2R | coagulation factor II (thrombin) receptor | Hs.128087 | NM_001992 | 3 |
| BNO529 | CTSB | cathepsin B | Hs.297939 | NM_001908 | 24 |
| BNO530 | LIF | leukemia inhibitory factor (cholinergic differentiation factor) | Hs.2250 | NM_002309 | 3 |
| BNO547 | EDN1 | endothelin 1 | Hs.2271 | NM_001955E | 0.5 |
| BNO550 | JAK1 | Janus kinase 1 (a protein tyrosine kinase) | Hs.50651 | NM_002227 | 24 |
| BNO563 | THBD | thrombomodulin | Hs.2030 | NM_000361 | 24 |
| BNO592 | PSEN1 | presenilin 1 (Alzheimer disease 3) | Hs.3260 | NM_000021 | 0.5 |
| BNO593 | STAT3 | signal transducer and activator of transcription 3 | Hs.321677 | NM_139276 | 6 |
| BNO601 | GJA1 | gap junction protein, alpha 1, 43 kDa (connexin 43) | Hs.74471 | NM_000165 | 3 |
| BNO608 | HEY1 | hairy/enhancer-of-split related with YRPW motif 1 | Hs.234434 | NM_012258 | 0.5 |
| BNO846 | CXCR4 | chemokine (C—X—C motif) receptor 4 | Hs.89414 | NM_003467 | 24 |
| BNO869 | ENTPD1 | ectonucleoside triphosphate diphosphohydrolase 1 | Hs.205353 | NM_001776 | 0.5 |
| BNO919 | SERPINE1 | serine (or cysteine) proteinase inhibitor, clade E, member 1 | Hs.82085 | NM_000602 | 3 |
| BNO923 | THBS1 | thrombospondin 1 | Hs.87409 | NM_003246 | 0.5 |

The invention also encompasses an isolated nucleic acid molecule that is at least 70% identical to any one of the angiogenic genes of the invention and which plays a role in the angiogenic process.

Such variants will have preferably at least about 85%, and most preferably at least about 95% sequence identity to the angiogenic genes. Any one of the polynucleotide variants described above can encode an amino acid sequence, which contains at least one functional or structural characteristic of the relevant angiogenic gene of the invention.

Sequence identity is typically calculated using the BLAST algorithm, described in Altschul et al (1997) with the BLOSUM62 default matrix.

The invention also encompasses an isolated nucleic acid molecule which hybridizes under stringent conditions with any one of the angiogenic genes of the invention and which plays a role in an angiogenic process.

Hybridization with PCR probes which are capable of detecting polynucleotide sequences, including genomic sequences, may be used to identify nucleic acid sequences which encode the relevant angiogenic gene. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding the angiogenic gene, allelic variants, or related sequences.

Probes may also be used for the detection of related sequences, and should preferably have at least 50% sequence identity to any of the angiogenic gene-encoding sequences of the invention. The hybridization probes of the present invention may be DNA or RNA and may be derived from any one of the angiogenic gene sequences or from genomic sequences including promoters, enhancers, and introns of the angiogenic genes.

Means for producing specific hybridization probes for DNAs encoding any one of the angiogenic genes include the cloning of polynucleotide sequences encoding the relevant angiogenic gene or its derivatives into vectors for the production of mRNA probes. Such vectors are known in the art, and are commercially available. Hybridization probes may be labelled by radionuclides such as ³²P or ³⁵S, or by enzymatic labels, such as alkaline phosphatase coupled to the probe via avidin/biotin coupling systems, or other methods known in the art.

Under stringent conditions, hybridization with ³²P labelled probes will most preferably occur at 42° C. in 750 mM NaCl, 75 mM trisodium citrate, 2% SDS, 50% formamide, 1×Denhart's, 10% (w/v) dextran sulphate and 100 μg/ml denatured salmon sperm DNA. Useful variations on these conditions will be readily apparent to those skilled in the art. The washing steps which follow hybridization most preferably occur at 65° C. in 15 mM NaCl, 1.5 mM trisodium citrate, and 1% SDS. Additional variations on these conditions will be readily apparent to those skilled in the art.

The nucleic acid molecules, or fragments thereof, of the present invention have a nucleotide sequence obtainable from a natural source. They therefore include naturally occurring normal, naturally occurring mutant, naturally occurring polymorphic alleles, differentially spliced transcripts, splice variants etc. Natural sources include animal cells and tissues, body fluids, tissue culture cells etc.

The nucleic acid molecules of the present invention can also be engineered using methods accepted in the art so as to alter the angiogenic gene-encoding sequences for a variety of purposes. These include, but are not limited to, modification of the cloning, processing, and/or expression of the gene product. PCR reassembly of gene fragments and the use of synthetic oligonucleotides allow the engineering of angiogenic gene nucleotide sequences. For example, oligonucleotide-mediated site-directed mutagenesis can introduce mutations that create new restriction sites, alter glycosylation patterns and produce splice variants etc.

As a result of the degeneracy of the genetic code, a number of nucleic acid sequences encoding the angiogenic genes of the invention, some that may have minimal similarity to the nucleic acid sequences of any known and naturally occurring gene, may be produced. Thus, the invention includes each and every possible variation of polynucleotide sequence that could be made by selecting combinations based on possible codon choices. These combinations are made in accordance with the standard triplet genetic code as applied to the polynucleotide sequence of the naturally occurring angiogenic gene, and all such variations are to be considered as being specifically disclosed.

The nucleic acid molecules of this invention are typically DNA molecules, and include cDNA, genomic DNA, synthetic forms, and mixed polymers, both sense and antisense strands, and may be chemically or biochemically modified, or may contain non-natural or derivatised nucleotide bases as will be appreciated by those skilled in the art. Such modifications include labels, methylation, intercalators, alkylators and modified linkages. In some instances it may be advantageous to produce nucleotide sequences encoding an angiogenic gene or its derivatives possessing a substantially different codon usage than that of the naturally occurring gene. For example, codons may be selected to increase the rate of expression of the peptide in a particular prokaryotic or eukaryotic host corresponding with the frequency that the host utilizes particular codons. Other reasons to alter the nucleotide sequence encoding an angiogenic gene or its derivatives without altering the encoded amino acid sequence include the production of RNA transcripts having more desirable properties, such as a greater half-life, than transcripts produced from the naturally occurring sequence.

The invention also encompasses production of the nucleic acid molecules of the invention, entirely by synthetic chemistry. Synthetic sequences may be inserted into expression vectors and cell systems that contain the necessary elements for transcriptional and translational control of the inserted coding sequence in a suitable host. These elements may include regulatory sequences, promoters, 5′ and 3′ untranslated regions and specific initiation signals (such as an ATG initiation codon and Kozak consensus sequence) which allow more efficient translation of sequences encoding the angiogenic genes. In cases where the complete coding sequence including its initiation codon and upstream regulatory sequences are inserted into the appropriate expression vector, additional control signals may not be needed. However, in cases where only coding sequence, or a fragment thereof, is inserted, exogenous translational control signals as described above should be provided by the vector. Such signals may be of various origins, both natural and synthetic. The efficiency of expression may be enhanced by the inclusion of enhancers appropriate for the particular host cell system used (Scharf et al., 1994).

The invention also includes nucleic acid molecules that are the complements of the sequences described herein.

The present invention allows for the preparation of purified polypeptides or proteins. In order to do this, host cells may be transfected with a nucleic acid molecule as described above. Typically, said host cells are transfected with an expression vector comprising a nucleic acid molecule according to the invention. A variety of expression vector/host systems may be utilized to contain and express the sequences. These include, but are not limited to, microorganisms such as bacteria transformed with plasmid or cosmid DNA expression vectors; yeast transformed with yeast expression vectors; insect cell systems infected with viral expression vectors (e.g., baculovirus); or mouse or other animal or human tissue cell systems. Mammalian cells can also be used to express a protein that is encoded by a specific angiogenic gene of the invention using various expression vectors including plasmid, cosmid and viral systems such as a vaccinia virus expression system. The invention is not limited by the host cell or vector employed.

The nucleic acid molecules, or variants thereof, of the present invention can be stably expressed in cell lines to allow long term production of recombinant proteins in mammalian systems. Sequences encoding any one of the angiogenic genes of the invention can be transformed into cell lines using expression vectors which may contain viral origins of replication and/or endogenous expression elements and a selectable marker gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

The protein produced by a transformed cell may be secreted or retained intracellularly depending on the sequence and/or the vector used. As will be understood by those of skill in the art, expression vectors containing polynucleotides which encode a protein may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of a “prepro” form of the protein may also be used to specify protein targeting, folding, and/or activity. Different host cells having specific cellular machinery and characteristic mechanisms for post-translational activities (e.g., CHO or HeLa cells), are available from the American Type Culture Collection (ATCC) and may be chosen to ensure the correct modification and processing of the foreign protein.

According to still another aspect of the present invention there is provided an expression vector comprising a nucleic acid molecule of the invention as described above.

According to still another aspect of the present invention there is provided a cell comprising a nucleic acid molecule of the invention as described above.

When large quantities of protein are needed such as for antibody production, vectors which direct high levels of expression may be used such as those containing the T5 or T7 inducible bacteriophage promoter. The present invention also includes the use of the expression systems described above in generating and isolating fusion proteins which contain important functional domains of the protein. These fusion proteins are used for binding, structural and functional studies as well as for the generation of appropriate antibodies.

In order to express and purify the protein as a fusion protein, the appropriate polynucleotide sequences of the present invention are inserted into a vector which contains a nucleotide sequence encoding another peptide (for example, glutathionine succinyl transferase). The fusion protein is expressed and recovered from prokaryotic or eukaryotic cells. The fusion protein can then be purified by affinity chromatography based upon the fusion vector sequence and the relevant protein can subsequently be obtained by enzymatic cleavage of the fusion protein.

Fragments of polypeptides of the present invention may also be produced by direct peptide synthesis using solid-phase techniques. Automated synthesis may be achieved by using the ABI 431A Peptide Synthesizer (Perkin-Elmer). Various fragments of polypeptide may be synthesized separately and then combined to produce the full length molecule.

In instances where the isolated nucleic acid molecules of the invention represent only partial gene sequence, these partial sequences can be used to obtain the corresponding sequence of the full-length angiogenic gene. Therefore, the present invention further provides the use of a partial nucleic acid molecule of the invention comprising a nucleotide sequence defined by any one of SEQ ID Numbers: 1 to 15, 17 to 37, and 39 to 44 to identify and/or obtain full-length human genes involved in the angiogenic process. Full-length angiogenic genes may be cloned using the partial nucleotide sequences of the invention by methods known per se to those skilled in the art. For example, in silico analysis of sequence databases such as those hosted at the National Centre for Biotechnology Information can be searched in order to obtain overlapping nucleotide sequence. This provides a “walking” strategy towards obtaining the full-length gene sequence. Appropriate databases to search at this site include the expressed sequence tag (EST) database (database of GenBank, EMBL and DDBJ sequences from their EST divisions) or the non redundant (nr) database (contains all GenBank, EMBL, DDBJ and PDB sequences but does not include EST, STS, OSS, or phase 0, 1 or 2 HTGS sequences). Typically searches are performed using the BLAST algorithm described in Altschul et al (1997) with the BLOSUM62 default matrix. In instances where in silico “walking” approaches fail to retrieve the complete gene sequence, additional strategies may be employed. These include the use of “restriction-site PCR” will allows the retrieval of unknown sequence adjacent to a portion of DNA whose sequence is known. In this technique universal primers are used to retrieve unknown sequence. Inverse PCR may also be used, in which primers based on the known sequence are designed to amplify adjacent unknown sequences. These upstream sequences may include promoters and regulatory elements. In addition, various other PCR-based techniques may be used, for example a kit available from Clontech (Palo Alto, Calif.) allows for a walking PCR technique, the 5′RACE kit (Gibco-BRL) allows isolation of additional 5′ gene sequence, while additional 3′ sequence can be obtained using practised techniques (for example see Gecz et al., 1997).

In a further aspect of the present invention there is provided an isolated polypeptide as defined by SEQ ID Numbers: 51 to 58 and laid out in Table 1.

The present invention also provides isolated polypeptides, which have been shown to be up-regulated in their expression during angiogenesis (see Tables 1 and 2).

More specifically, following the realisation that these polypeptides are up-regulated in their expression during angiogenesis, the invention provides isolated polypeptides as defined by SEQ ID Numbers: 51 to 58, and as laid out in Tables 1 and 2, or fragments thereof, that play a role in an angiogenic process. Such a process may include, but is not restricted to, embryogenesis, menstrual cycle, wound repair, tumour angiogenesis and exercise induced muscle hypertrophy.

In addition, the present invention provides isolated polypeptides as defined by SEQ ID Numbers: 51 to 58, and as laid out in Tables 1 and 2, or fragments thereof, that play a role in diseases associated with the angiogenic process. Diseases may include, but are not restricted to, cancer, rheumatoid arthritis, diabetic retinopathy, psoriasis, and cardiovascular diseases such as atherosclerosis, ischaemic limb disease and coronary artery disease.

The invention also encompasses an isolated polypeptide having at least 70%, preferably 85%, and more preferably 95%, identity to any one of SEQ ID Numbers: 51 to 58, and which plays a role in an angiogenic process.

Sequence identity is typically calculated using the BLAST algorithm, described in Altschul et al (1997) with the BLOSUM62 default matrix.

In a further aspect of the invention there is provided a method of preparing a polypeptide as described above, comprising the steps of:

(1) culturing cells as described above under conditions effective for production of the polypeptide; and

(2) harvesting the polypeptide.

According to still another aspect of the invention there is provided a polypeptide which is the product of the process described above.

Substantially purified protein or fragments thereof can then be used in further biochemical analyses to establish secondary and tertiary structure. Such methodology is known in the art and includes, but is not restricted to, X-ray crystallography of crystals of the proteins or by nuclear magnetic resonance (NMR). Determination of structure allows for the rational design of pharmaceuticals to interact with the protein, alter protein charge configuration or charge interaction with other proteins, or to alter its function in the cell.

The invention has provided a number of genes likely to be involved in angiogenesis and therefore enables methods for the modulation of angiogenesis. As angiogenesis is critical in a number of pathological processes, the invention therefore also enables therapeutic methods for the treatment of all angiogenesis-related disorders, and may enable the diagnosis or prognosis of all angiogenesis-related disorders associated with abnormalities in expression and/or function of any one of the angiogenic genes.

Examples of such disorders include, but are not limited to, cancer, rheumatoid arthritis, diabetic retinopathy, psoriasis, and cardiovascular diseases such as atherosclerosis, ischaemic limb disease and coronary artery disease.

### Therapeutic Applications

According to another aspect of the present invention there is provided a method of treating an angiogenesis-related disorder as described above, comprising administering a selective antagonist or agonist of an angiogenic gene or protein of the invention to a subject in need of such treatment.

In still another aspect of the invention there is provided the use of a selective antagonist or agonist of an angiogenic gene or protein of the invention in is the manufacture of a medicament for the treatment of an angiogenesis-related disorder as described above.

For the treatment of angiogenesis-related disorders which result in uncontrolled or enhanced angiogenesis, including but not limited to, cancer, rheumatoid arthritis, diabetic retinopathy, psoriasis and cardiovascular diseases such as atherosclerosis, therapies which inhibit the expanding vasculature are desirable. This would involve inhibition of any one of the angiogenic genes or proteins that are able to promote angiogenesis, or enhancement, stimulation or re-activation of any one of the angiogenic genes or proteins that are able to inhibit angiogenesis.

For the treatment of angiogenesis-related disorders which are characterised by inhibited or decreased angiogenesis, including but not limited to, ischaemic limb disease and coronary artery disease, therapies which enhance or promote vascular expansion are desirable. This would involve inhibition of any one of the angiogenic genes or proteins that are able to restrict angiogenesis or enhancement, stimulation or re-activation of any one of the angiogenic genes or proteins that are able to promote angiogenesis.

For instance, decreasing the expression of BNO782 and BNO481 has been shown to disrupt endothelial cell activity leading to an inhibition of capillary tube formation and angiogenesis. Therefore, in the treatment of disorders where angiogenesis needs to be restricted, it would be desirable to inhibit the function of these genes. Alternatively, in the treatment of disorders where angiogenesis needs to be stimulated it may be desirable to enhance the function of these genes.

In some embodiments, a method of modulating angiogenesis comprising modulating the expression or activity of a BNO802 polypeptide in a cell, wherein the BNO802 polypeptide is encoded by a BNO802 nucleic acid molecule set forth in Table 1, is provided.

For each of these cases, the relevant therapy will be useful in treating angiogenesis-related disorders regardless of whether there is a lesion in the angiogenic gene.

### Inhibiting Gene or Protein Function

Inhibiting the function of a gene or protein can be achieved in a variety of ways. Antisense nucleic acid methodologies represent one approach to inactivate genes that are causative of a disorder. Antisense or gene-targeted silencing strategies may include, but are not limited to, the use of antisense oligonucleotides, injection of antisense RNA, transfection of antisense RNA expression vectors, and the use of RNA interference (RNAi) or short interfering RNAs (siRNA). RNAi can be used in vitro and in vivo to silence a gene when its expression contributes to angiogenesis (Sharp and Zamore, 2000; Grishok et al., 2001). Still further, catalytic nucleic acid molecules such as DNAzymes and ribozymes may be used for gene silencing (Breaker and Joyce, 1994; Haseloff and Gerlach, 1988). These molecules function by cleaving their target mRNA molecule rather than merely binding to it as in traditional antisense approaches.

In one aspect of the invention an isolated nucleic acid molecule, which is the complement of any one of the relevant angiogenic nucleic acid molecules described above may be administered to a subject in need of such treatment. Typically, a complement to any relevant one of the angiogenic genes is administered to a subject to treat or prevent an angiogenesis-related disorder. In a further aspect the complement may encode an RNA molecule that hybridizes with the mRNA encoded by the relevant angiogenic gene of the invention or may be a short interfering oligonucleotide (siRNA) that hybridizes with the mRNA encoded by the relevant angiogenic gene of the invention.

In a further aspect of the invention there is provided the use of an isolated nucleic acid molecule which is the complement of any one of the relevant nucleic acid molecules of the invention and which encodes an RNA molecule or a short interfering oligonucleotide (siRNA) that hybridizes with the mRNA encoded by the relevant angiogenic gene of the invention, in the manufacture of a medicament for the treatment of an angiogenesis-related disorder.

Typically, a vector expressing the complement of a polynucleotide encoding any one of the relevant angiogenic genes may be administered to a subject to treat or prevent an angiogenesis-related disorder including, but not limited to, those described above. Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (For example, see Goldman et al., 1997).

In a further aspect purified protein according to the invention may be used to produce antibodies which specifically bind any relevant angiogenic protein of the invention. These antibodies may be used directly as an antagonist or indirectly as a targeting or delivery mechanism for bringing a pharmaceutical agent (such as a cytotoxic agent) to cells or tissues that express the relevant angiogenic protein. Such antibodies may include, but are not limited to, polyclonal, monoclonal, chimeric and single chain antibodies as would be understood by the person skilled in the art.

For the production of antibodies, various hosts including rabbits, rats, goats, mice, humans, and others may be immunized by injection with a protein of the invention or with any fragment or oligopeptide thereof, which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum.

It is preferred that the oligopeptides, peptides, or fragments used to induce antibodies to the relevant angiogenic protein have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of amino acids from these proteins may be fused with those of another protein, such as KLH, and antibodies to the chimeric molecule may be produced.

Monoclonal antibodies to any relevant angiogenic protein may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler and Milstein, 1975; Kozbor et al., 1985; Cote et al., 1983; Cole et al., 1984).

Monoclonal antibodies produced may include, but are not limited to, mouse-derived antibodies, humanised antibodies and fully-human antibodies. For example, antibodies are obtained from transgenic mice that have been engineered to produce specific human antibodies in response to antigenic challenge. In one example of this technique, elements of the human heavy and light chain loci are introduced into strains of mice derived from embryonic stem cell lines that contain targeted disruptions of the endogenous heavy and light chain loci. These transgenic mice can synthesise human antibodies specific for human antigens and can be used to produce human antibody-secreting hybridomas. Methods for obtaining human antibodies from transgenic mice are described for example in Lonberg et al., 1994; Green et al., 1994; Taylor et al., 1994.

Antibodies may also be produced by inducing in vivo production in the lymphocyte population or by screening immunoglobulin libraries or panels of highly specific binding reagents as disclosed in the literature. (For example, see Orlandi et al., 1989; Winter et al., 1991).

Antibody fragments which contain specific binding sites for any relevant angiogenic protein may also be generated. For example, such fragments include, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (For example, see Huse et al., 1989).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between a protein and its specific antibody. A two-site, monoclonal-based immunoassay utilizing monoclonal antibodies reactive to two non-interfering epitopes is preferred, but a competitive binding assay may also be employed.

In a further aspect, antagonists may include peptides, phosphopeptides or small organic or inorganic compounds. These antagonists should disrupt the function of any relevant angiogenic gene of the invention so as to provide the necessary therapeutic effect.

Peptides, phosphopeptides or small organic or inorganic compounds suitable for therapeutic applications may be identified using nucleic acids and polypeptides of the invention in drug screening applications as described below.

### Enhancing Gene or Protein Function

Enhancing, stimulating or re-activating a gene's or protein's function can be achieved in a variety of ways. In one aspect of the invention administration of an isolated nucleic acid molecule, as described above, to a subject in need of such treatment may be initiated. Typically, any relevant angiogenic gene of the invention can be administered to a subject to treat or prevent an angiogenesis-related disorder.

In a further aspect, there is provided the use of an isolated nucleic acid molecule, as described above, in the manufacture of a medicament for the treatment of an angiogenesis-related disorder.

Typically, a vector capable of expressing any relevant angiogenic gene, or a fragment or derivative thereof, may be administered to a subject to treat or prevent a disorder including, but not limited to, those described above. Transducing retroviral vectors are often used for somatic cell gene therapy because of their high efficiency of infection and stable integration and expression. Any relevant full-length gene, or portions thereof, can be cloned into a retroviral vector and expression may be driven from its endogenous promoter or from the retroviral long terminal repeat or from a promoter specific for the target cell type of interest. Other viral vectors can be used and include, as is known in the art, adenoviruses, adeno-associated viruses, vaccinia viruses, papovaviruses, lentiviruses and retroviruses of avian, murine and human origin.

Gene therapy would be carried out according to established methods (Friedman, 1991; Culver, 1996). A vector containing a copy of any relevant angiogenic gene linked to expression control elements and capable of replicating inside the cells is prepared. Alternatively the vector may be replication deficient and may require helper cells for replication and use in gene therapy.

Gene transfer using non-viral methods of infection in vitro can also be used. These methods include direct injection of DNA, uptake of naked DNA in the presence of calcium phosphate, electroporation, protoplast fusion or liposome delivery. Gene transfer can also be achieved by delivery as a part of a human artificial chromosome or receptor-mediated gene transfer. This involves linking the DNA to a targeting molecule that will bind to specific cell-surface receptors to induce endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes.

Although not identified to date, it is possible that certain individuals with angiogenesis-related disorders contain an abnormality in any one of the angiogenic genes of the invention. In affected subjects that express a mutated form of any one of the angiogenic genes of the invention it may be possible to prevent the disorder by introducing into the affected cells a wild-type copy of the gene such that it recombines with the mutant gene. This requires a double recombination event for the correction of the gene mutation. Vectors for the introduction of genes in these ways are known in the art, and any suitable vector may be used. Alternatively, introducing another copy of the gene bearing a second mutation in that gene may be employed so as to negate the original gene mutation and block any negative effect.

In a still further aspect, there is provided a method of treating an angiogenesis-related disorder comprising administering a polypeptide, as described above, or an agonist thereof, to a subject in need of such treatment.

In another aspect the invention provides the use of a polypeptide as described above, or an agonist thereof, in the manufacture of a medicament for the treatment of an angiogenesis-related disorder. Examples of such disorders are described above.

In a further aspect, a suitable agonist may also include peptides, phosphopeptides or small organic or inorganic compounds that can mimic the function of any relevant angiogenic gene, or may include an antibody to any relevant angiogenic gene that is able to restore function to a normal level.

Peptides, phosphopeptides or small organic or inorganic compounds suitable for therapeutic applications may be identified using nucleic acids and polypeptides of the invention in drug screening applications as described below.

In further embodiments, any of the agonists, antagonists, complementary sequences, nucleic acid molecules, proteins, antibodies, or vectors of the invention may be administered in combination with other appropriate therapeutic agents. Selection of the appropriate agents may be made by those skilled in the art, according to conventional pharmaceutical principles. The combination of therapeutic agents may act synergistically to effect the treatment or prevention of the various disorders described above. Using this approach, therapeutic efficacy with lower dosages of each agent may be possible, thus reducing the potential for adverse side effects.

Any of the therapeutic methods described above may be applied to any subject in need of such therapy, including, for example, mammals such as dogs, cats, cows, horses, rabbits, monkeys, and most preferably, humans.

### Modulation of Angiogenesis

As the invention has provided a number of genes likely to be involved in angiogenesis it therefore enables methods for the modulation of angiogenesis. In a further aspect of the present invention, any of the methods described above used for the treatment of an angiogenesis-related disorder may be used for the modulation of angiogenesis in any system comprising cells. These systems may include but are not limited to, in vitro assay systems (e.g. Matrigel assays, proliferation assays, migration assays, collagen assays, bovine capillary endothelial cell assay etc), in vivo assay systems (e.g. in vivo Matrigel-type assays, chicken chorioallantoic membrane assay, isolated organs, tissues or cells etc), animal models (e.g. in vivo neovascularisation assays, tumour angiogenesis models etc) or hosts in need of treatment (e.g. hosts suffering from angiogenesis-related disorders as previously described.

### Drug Screening

According to still another aspect of the invention, nucleic acid molecules of the invention as well as peptides of the invention, particularly any relevant purified angiogenic polypeptides or fragments thereof, and cells expressing these are useful for screening of candidate pharmaceutical compounds in a variety of techniques for the treatment of angiogenesis-related disorders.

Still further, it provides the use wherein high throughput screening techniques are employed.

Compounds that can be screened in accordance with the invention include, but are not limited to peptides (such as soluble peptides), phosphopeptides and small organic or inorganic molecules (such as natural product or synthetic chemical libraries and peptidomimetics).

In one embodiment, a screening assay may include a cell-based assay utilising eukaryotic or prokaryotic host cells that are stably transformed with recombinant nucleic acid molecules expressing the relevant angiogenic polypeptide or fragment, in competitive binding assays. Binding assays will measure for the formation of complexes between the relevant polypeptide or fragments thereof and the compound being tested, or will measure the degree to which a compound being tested will interfere with the formation of a complex between the relevant polypeptide or fragment thereof, and its interactor or ligand.

Non cell-based assays may also be used for identifying compounds that interrupt binding between the polypeptides of the invention and their interactors. Such assays are known in the art and include for example AlphaScreen technology (PerkinElmer Life Sciences, MA, USA). This application relies on the use of beads such that each interaction partner is bound to a separate bead via an antibody. Interaction of each partner will bring the beads into proximity, such that laser excitation initiates a number of chemical reactions ultimately leading to fluorophores emitting a light signal. Candidate compounds that disrupt the binding of the relevant angiogenic polypeptide with its interactor will result in loss of light emission enabling identification and isolation of the responsible compound.

High-throughput drug screening techniques may also employ methods as described in WO84/03564. Small peptide test compounds synthesised on a solid substrate can be assayed through relevant angiogenic polypeptide binding and washing. The relevant bound angiogenic polypeptide is then detected by methods well known in the art. In a variation of this technique, purified angiogenic polypeptides can be coated directly onto plates to identify interacting test compounds.

An additional method for drug screening involves the use of host eukaryotic cell lines that carry mutations in any relevant angiogenic gene of the invention. The host cell lines are also defective at the polypeptide level. Other cell lines may be used where the expression of the relevant angiogenic gene can be regulated (i.e. over-expressed, under-expressed, or switched off). The host cell lines or cells are grown in the presence of various drug compounds and the rate of growth of the host cells is measured to determine if the compound is capable of regulating the growth of defective cells.

The angiogenic polypeptides of the present invention may also be used for screening compounds developed as a result of combinatorial library technology. This provides a way to test a large number of different substances for their ability to modulate activity of a polypeptide. A substance identified as a modulator of polypeptide function may be peptide or non-peptide in nature. Non-peptide “small molecules” are often preferred for many in vivo pharmaceutical applications. In addition, a mimic or mimetic of the substance may be designed for pharmaceutical use. The design of mimetics based on a known pharmaceutically active compound (“lead” compound) is a common approach to the development of novel pharmaceuticals. This is often desirable where the original active compound is difficult or expensive to synthesise or where it provides an unsuitable method of administration. In the design of a mimetic, particular parts of the original active compound that are important in determining the target property are identified. These parts or residues constituting the active region of the compound are known as its pharmacophore. Once found, the pharmacophore structure is modelled according to its physical properties using data from a range of sources including x-ray diffraction data and NMR. A template molecule is then selected onto which chemical groups that mimic the pharmacophore can be added. The selection can be made such that the mimetic is easy to synthesise, is likely to be pharmacologically acceptable, does not degrade in vivo and retains the biological activity of the lead compound. Further optimisation or modification can be carried out to select one or more final mimetics useful for in vivo or clinical testing.

It is also possible to isolate a target-specific antibody and then solve its crystal structure. In principle, this approach yields a pharmacophore upon which subsequent drug design can be based as described above. It may be possible to avoid protein crystallography altogether by generating anti-idiotypic antibodies (anti-ids) to a functional, pharmacologically active antibody. As a mirror image of a mirror image, the binding site of the anti-ids would be expected to be an analogue of the original binding site. The anti-id could then be used to isolate peptides from chemically or biologically produced peptide banks.

Another alternative method for drug screening relies on structure-based rational drug design. Determination of the three dimensional structure of the polypeptides of the invention, or the three dimensional structure of the protein complexes which may incorporate these polypeptides allows for structure-based drug design to identify biologically active lead compounds.

Three dimensional structural models can be generated by a number of applications, some of which include experimental models such as x-ray crystallography and NMR and/or from in silico studies using information from structural databases such as the Protein Databank (PDB). In addition, three dimensional structural models can be determined using a number of known protein structure prediction techniques based on the primary sequences of the polypeptides (e.g. SYBYL—Tripos Associated, St. Louis, Mo.), de novo protein structure design programs (e.g. MODELER—MSI Inc., San Diego, Calif., or MOE—Chemical Computing Group, Montreal, Canada) or ab initio methods (e.g. see U.S. Pat. Nos. 5,331,573 and 5,579,250).

Once the three dimensional structure of a polypeptide or polypeptide complex has been determined, structure-based drug discovery techniques can be employed to design biologically active compounds based on these three dimensional structures. Such techniques are known in the art and include examples such as DOCK (University of California, San Francisco) or AUTODOCK (Scripps Research Institute, La Jolla, Calif.). A computational docking protocol will identify the active site or sites that are deemed important for protein activity based on a predicted protein model. Molecular databases, such as the Available Chemicals Directory (ACD) are then screened for molecules that complement the protein model.

Using methods such as these, potential clinical drug candidates can be identified and computationally ranked in order to reduce the time and expense associated with typical ‘wet lab’ drug screening methodologies.

Compounds identified from the screening methods described above form a part of the present invention, as do pharmaceutical compositions containing these and a pharmaceutically acceptable carrier.

### Pharmaceutical Preparations

Compounds identified from screening assays as indicated above can be administered to a patient at a therapeutically effective dose to treat or ameliorate a disorder associated with angiogenesis. A therapeutically effective dose refers to that amount of the compound sufficient to result in amelioration of symptoms of the disorder.

Toxicity and therapeutic efficacy of such compounds can be determined by standard pharmaceutical procedures in cell cultures or experimental animals. The data obtained from these studies can then be used in the formulation of a range of dosages for use in humans.

Pharmaceutical compositions for use in accordance with the present invention can be formulated in a conventional manner using one or more physiological acceptable carriers, excipients or stabilisers which are well known. Acceptable carriers, excipients or stabilizers are non-toxic at the dosages and concentrations employed, and include buffers such as phosphate, citrate, and other organic acids; antioxidants including absorbic acid; low molecular weight (less than about 10 residues) polypeptides; proteins, such as serum albumin, gelatin, or immunoglobulins; binding agents including hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or non-ionic surfactants such as Tween, Pluronics or polyethylene glycol (PEG).

The formulation of pharmaceutical compositions for use in accordance with the present invention will be based on the proposed route of administration. Routes of administration may include, but are not limited to, inhalation, insufflation (either through the mouth or nose), oral, buccal, rectal or parental administration.

### Diagnostic and Prognostic Applications

Should abnormalities in any one of the angiogenic genes of the invention exist, which alter activity and/or expression of the gene to give rise to angiogenesis-related disorders, the polynucleotides and polypeptides of the invention may be used for the diagnosis or prognosis of these disorders, or a predisposition to such disorders. Examples of such disorders include, but are not limited to, cancer, rheumatoid arthritis, diabetic retinopathy, psoriasis, cardiovascular diseases such as atherosclerosis, ischaemic limb disease and coronary artery disease. Diagnosis or prognosis may be used to determine the severity, type or stage of the disease state in order to initiate an appropriate therapeutic intervention.

In another embodiment of the invention, the polynucleotides that may be used for diagnostic or prognostic purposes include oligonucleotide sequences, genomic DNA and complementary RNA and DNA molecules. The polynucleotides may be used to detect and quantitate gene expression in biopsied tissues in which abnormal expression or mutations in any one of the angiogenic genes may be correlated with disease. Genomic DNA used for the diagnosis or prognosis may be obtained from body cells, such as those present in the blood, tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, direct nucleotide sequencing, reverse transcriptase PCR (RT-PCR), hybridization using specific oligonucleotides, restriction enzyme digest and mapping, PCR mapping, RNAse protection, and various other methods may be employed. Oligonucleotides specific to particular sequences can be chemically synthesized and labelled radioactively or nonradioactively and hybridized to individual samples immobilized on membranes or other solid-supports or in solution. The presence, absence or excess expression of any one of the angiogenic genes may then be visualized using methods such as autoradiography, fluorometry, or colorimetry.

In a particular aspect, the nucleotide sequences of the invention may be useful in assays that detect the presence of associated disorders, particularly those mentioned previously. The nucleotide sequences may be labelled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to a control sample then the presence of altered levels of nucleotide sequences in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis or prognosis of an angiogenesis-related disorder associated with a mutation in any one of the angiogenic genes of the invention, the nucleotide sequence of the relevant gene can be compared between normal tissue and diseased tissue in order to establish whether the patient expresses a mutant gene.

In order to provide a basis for the diagnosis or prognosis of a disorder associated with abnormal expression of any one of the angiogenic genes of the invention, a normal or standard profile for expression is established. This may be accomplished by combining body fluids or cell extracts taken from normal subjects, either animal or human, with a sequence, or a fragment thereof, encoding the relevant angiogenic gene, under conditions suitable for hybridization or amplification. Standard hybridization may be quantified by comparing the values obtained from normal subjects with values from an experiment in which a known amount of a substantially purified polynucleotide is used. Another method to identify a normal or standard profile for expression of any one of the angiogenic genes is through quantitative RT-PCR studies. RNA isolated from body cells of a normal individual, particularly RNA isolated from endothelial cells, is reverse transcribed and real-time PCR using oligonucleotides specific for the relevant gene is conducted to establish a normal level of expression of the gene. Standard values obtained in both these examples may be compared with values obtained from samples from patients who are symptomatic for a disorder. Deviation from standard values is used to establish the presence of a disorder.

Once the presence of a disorder is established and a treatment protocol is initiated, hybridization assays or quantitative RT-PCR studies may be repeated on a regular basis to determine if the level of expression in the patient begins to approximate that which is observed in the normal subject. The results obtained from successive assays may be used to show the efficacy of treatment over a period ranging from several days to months.

According to a further aspect of the invention there is provided the use of an angiogenic polypeptide as described above in the diagnosis or prognosis of an angiogenesis-related disorder associated with any one of angiogenic genes of the invention, or a predisposition to such disorders.

When a diagnostic or prognostic assay is to be based upon any relevant angiogenic polypeptide, a variety of approaches are possible. For example, diagnosis or prognosis can be achieved by monitoring differences in the electrophoretic mobility of normal and mutant proteins. Such an approach will be particularly useful in identifying mutants in which charge substitutions are present, or in which insertions, deletions or substitutions have resulted in a significant change in the electrophoretic migration of the resultant protein. Alternatively, diagnosis or prognosis may be based upon differences in the proteolytic cleavage patterns of normal and mutant proteins, differences in molar ratios of the various amino acid residues, or by functional assays demonstrating altered function of the gene products.

In another aspect, antibodies that specifically bind the relevant angiogenic gene product may be used for the diagnosis or prognosis of disorders characterized by abnormal expression of the gene, or in assays to monitor patients being treated with the relevant angiogenic gene or protein or agonists, antagonists, or inhibitors thereof. Antibodies useful for diagnostic or prognostic purposes may be prepared in the same manner as described above for therapeutics. Diagnostic or prognostic assays may include methods that utilize the antibody and a label to detect the relevant protein in human body fluids or in extracts of cells or tissues. The antibodies may be used with or without modification, and may be labelled by covalent or non-covalent attachment of a reporter molecule.

A variety of assays for measuring the relevant angiogenic polypeptide based on the use of antibodies specific for the polypeptide are known in the art and provide a basis for diagnosing altered or abnormal levels of expression. Normal or standard values for expression are established by combining body fluids or cell extracts taken from normal mammalian subjects, preferably human, with antibody to the relevant protein under conditions suitable for complex formation. The amount of standard complex formation may be quantitated by various methods which are known in the art. Examples include, but are not limited to, enzyme-linked immunosorbent assays (ELISAs), radioimmunoassays (RIAs), immunofluorescence, flow cytometry, histology, electron microscopy, in situ assays, immunoprecipitation, Western blot etc. For example, using the ELISA technique an enzyme which is bound to the antibody will react with an appropriate substrate, preferably a chromogenic substrate, in such a manner as to produce a chemical moiety that can be detected for example by spectrophotomeric, fluorimetric or by visual means. Detection may also be accomplished by using other assays such as RIAs where the antibodies or antibody fragments are radioactively labelled. It is also possible to label the antibody with a fluorescent compound. When the fluorescently labelled antibody is exposed to light of a certain wavelength, its presence can then be detected due to fluorescence. The antibody can also be detectably labelled by coupling it to a chemiluminescent compound. The presence of the chemiluminescent-tagged antibody is then determined by detecting the presence of luminescence that arises during the course of a chemical reaction.

Quantities of protein expressed in subject, control, and disease samples from biopsied tissues are compared with the standard values. Deviation between standard and subject values establishes the parameters for diagnosing or prognosing disease.

Once an individual has been diagnosed or prognosed with a disorder, effective treatments can be initiated, as described above. In the treatment of angiogenesis-related diseases which are characterised by uncontrolled or enhanced angiogenesis, the expanding vasculature needs to be inhibited. This would involve inhibiting the relevant angiogenic genes or proteins of the invention that promote angiogenesis. In addition, treatment may also need to stimulate expression or function of the relevant angiogenic genes or proteins of the invention whose normal role is to inhibit angiogenesis but whose activity is reduced or absent in the affected individual.

In the treatment of angiogenesis-related diseases which are characterised by inhibited or decreased angiogenesis, approaches which enhance or promote vascular expansion are desirable. This may be achieved using methods essentially as described above but will involve stimulating the expression or function of the relevant angiogenic gene or protein whose normal role is to promote angiogenesis but whose activity is reduced or absent in the affected individual. Alternatively, inhibiting genes or proteins that restrict angiogenesis may also be an approach to treatment.

### Microarray

In further embodiments, complete cDNAs, oligonucleotides or longer fragments derived from any of the polynucleotide sequences described herein may be used as probes in a microarray. The microarray can be used to monitor the expression level of large numbers of genes simultaneously and to identify genetic variants, mutations, and polymorphisms. This information may be used to determine gene function, to understand the genetic basis of angiogenesis-related disorders, to diagnose or prognose angiogenesis-related disorders, and to develop and monitor the activities of therapeutic agents. Microarrays may be prepared, used, and analysed using methods known in the art. (For example, see Schena et al., 1996; Heller et al., 1997).

### Transformed Hosts

The present invention also provides for the production of genetically modified (knock-out, knock-in and transgenic), non-human animal models comprising the nucleic acid molecules of the invention. These animals are useful for the study of the function of the relevant angiogenic gene, to study the process of angiogenesis, to study the mechanisms of angiogenic disease as related to these genes, for the screening of candidate pharmaceutical compounds for the treatment of angiogenesis-related disorders for the creation of explanted mammalian cell cultures which express the protein or mutant protein, and for the evaluation of potential therapeutic interventions.

Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, genetically modified mice and rats are highly desirable due to the relative ease in generating knock-in, knock-out or transgenics of these animals, their ease of maintenance and their shorter life spans. For certain studies, transgenic yeast or invertebrates may be suitable and preferred because they allow for rapid screening and provide for much easier handling. For longer term studies, non-human primates may be desired due to their similarity with humans.

To create an animal model based on any one of the angiogenic genes of the invention, several methods can be employed. These include, but are not limited to, generation of a specific mutation in a homologous animal gene, insertion of a wild type human gene and/or a humanized animal gene by homologous recombination, insertion of a mutant (single or multiple) human gene as genomic or minigene cDNA constructs using wild type, mutant or artificial promoter elements, or insertion of artificially modified fragments of the endogenous gene by homologous recombination. The modifications include insertion of mutant stop codons, the deletion of DNA sequences, or the inclusion of recombination elements (lox p sites) recognized by enzymes such as Cre recombinase.

To create transgenic mice in order to study gain of gene function in vivo, any relevant angiogenic gene can be inserted into a mouse germ line using standard techniques such as oocyte microinjection. Gain of gene function can mean the overexpression of a gene and its protein product, or the genetic complementation of a mutation of the gene under investigation. For oocyte injection, one or more copies of the wild type or mutant gene can be inserted into the pronucleus of a just-fertilized mouse oocyte. This oocyte is then reimplanted into a pseudo-pregnant foster mother. The liveborn mice can then be screened for integrants using analysis of tail DNA for the presence of the relevant human angiogenic gene sequence. The transgene can be either a complete genomic sequence injected as a YAC, BAC, PAC or other chromosome DNA fragment, a cDNA with either the natural promoter or a heterologous promoter, or a minigene containing all of the coding region and other elements found to be necessary for optimum expression.

To generate knock-out mice or knock-in mice, gene targeting through homologous recombination in mouse embryonic stem (ES) cells may be applied. Knock-out mice are generated to study loss of gene function in vivo while knock-in mice allow the study of gain of function or to study the effect of specific gene mutations. Knock-in mice are similar to transgenic mice however the integration site and copy number are defined in the former.

For knock-out mouse generation, gene targeting vectors can be designed such that they disrupt (knock-out) the protein coding sequence of the relevant angiogenic gene in the mouse genome. Knock-out animals of the invention will comprise a functional disruption of a relevant angiogenesis gene of the invention such that the gene does not express a biologically active product. It can be substantially deficient in at least one functional activity coded for by the gene. Expression of the polypeptide encoded by the gene can be substantially absent (i.e. essentially undetectable amounts are made) or may be deficient in activity such as where only a portion of the gene product is produced. In contrast, knock-in mice can be produced whereby a gene targeting vector containing the relevant angiogenic gene can integrate into a defined genetic locus in the mouse genome. For both applications, homologous recombination is catalysed by specific DNA repair enzymes that recognise homologous DNA sequences and exchange them via double crossover.

Gene targeting vectors are usually introduced into ES cells using electroporation. ES cell integrants are then isolated via an antibiotic resistance gene present on the targeting vector and are subsequently genotyped to identify those ES cell clones in which the gene under investigation has integrated into the locus of interest. The appropriate ES cells are then transmitted through the germline to produce a novel mouse strain.

In instances where gene ablation results in early embryonic lethality, conditional gene targeting may be employed. This allows genes to be deleted in a temporally and spatially controlled fashion. As above, appropriate ES cells are transmitted through the germline to produce a novel mouse strain, however the actual deletion of the gene is performed in the adult mouse in a tissue specific or time controlled manner. Conditional gene targeting is most commonly achieved by use of the cre/lox system. The enzyme cre is able to recognise the 34 base pair loxP sequence such that loxP flanked (or floxed) DNA is recognised and excised by cre. Tissue specific cre expression in transgenic mice enables the generation of tissue specific knock-out mice by mating gene targeted floxed mice with cre transgenic mice. Knock-out can be conducted in every tissue (Schwenk et al., 1995) using the ‘deleter’ mouse or using transgenic mice with an inducible cre gene (such as those with tetracycline inducible cre genes), or knock-out can be tissue specific for example through the use of the CD19-cre mouse (Rickert et al., 1997).

According to still another aspect of the invention there is provided the use of genetically modified non-human animals for the screening of candidate pharmaceutical compounds.

It will be clearly understood that, although a number of prior art publications are referred to herein, this reference does not constitute an admission that any of these documents forms part of the common general knowledge in the art, in Australia or in any other country. Throughout this specification and the claims, the words “comprise”, “comprises” and “comprising” are used in a non-exclusive sense, except where the context requires otherwise.

### EXAMPLES

### Example 1

### In Vitro Capillary Tube Formation

The in vitro model of angiogenesis is essentially as described in Gamble et al (1993). The assay was performed in collagen under the stimulation of phorbol myristate acetate (PMA) and the anti-integrin (α₂β₁) antibody, RMACII. Human umbilical vein endothelial cells (HUVECs) were used in all experiments between passages 2 to 4.

Cells were harvested from bulk cultures (t=0), replated onto the collagen gels with stimulation and then harvested from the collagen gels at 0.5, 3.0, 6.0 and 24 hours after commencement of the assay. These time points were chosen since major morphological changes occur at these stages. Briefly, by 0.5 hours, cells have attached to the collagen matrix and have commenced migration into the gel. By 3.0 hours, small intracellular vesicles are visible. By 6.0 hours, these vesicles are coalescing together to form membrane bound vacuoles and the cells in the form of short sprouts have invaded the gel. After this time, these vacuoles fuse with the plasma membrane, thus expanding the intercellular space to generate the lumen (Meyer et al., 1997). The formation of these larger vacuoles is an essential requirement of lumen formation (Gamble et al., 1999). By 24 hours, the overall anastomosing network of capillary tubes has formed and has commenced degeneration.

### Example 2

### RNA Isolation, cDNA Synthesis and Amplification

Cells harvested at the specified time points were used for the isolation of total RNA using the Trizol reagent (Gibco BRL) according to manufacturers conditions. SMART (Switching mechanism at 5′ end of RNA transcript) technology was used to convert small amounts of total RNA into enough cDNA to enable cDNA subtraction to be performed (see below). This was achieved using the SMART-PCR cDNA synthesis kit (Clontech-user manual PT3041-1) according to manufacturers recommendations. The SMART-PCR cDNA synthesis protocol generated a majority of full length cDNAs which were subsequently PCR amplified for cDNA subtraction.

### Example 3

### Suppression Subtractive Hybridization (SSH)

SSH was performed on SMART amplified cDNA in order to enrich for cDNAs that were either up-regulated or down-regulated between the cDNA populations defined by the selected time-points. This technique also allowed “normalisation” of the regulated cDNAs, thereby making low abundance cDNAs (i.e. poorly expressed, but important, genes) more easily detectable. To do this, the PCR-Select cDNA synthesis kit (Clontech-user manual PT3041-1) and PCR-Select cDNA subtraction kit (Clontech-user manual PT1117-1) were used based on manufacturers conditions. These procedures relied on subtractive hybridization and suppression PCR amplification. SSH was performed between the following populations: 0-0.5 hours; 0.5-3.0 hours; 3.0-6.0 hours; 6.0-24 hours.

### Example 4

### Differential Screening of cDNA Clones

Following SSH, the cDNA fragments were digested with EagI and cloned into the compatible unique NotI site in pBluescript KS⁺ using standard techniques (Sambrook et al., 1989). This generated forward and reverse subtracted libraries for each time period. Initially, the forward subtracted libraries were used in subsequent studies to identify those clones representing genes that were up-regulated in their expression during the in vitro model of angiogenesis. To do this, a microarray analysis procedure was adopted.

### Microarray Slide Preparation

A total of 10,000 clones from the 4 forward subtracted libraries (3,200 clones from 0-0.5 hr; 3,000 clones from 0.5-3 hr; 2,800 clones from 3-6 hr; 1,000 clones from 6-24 hr) were chosen to construct microarray slides. Inserts from these clones were amplified using standard PCR techniques with flanking T3 and T7 pBluescript KS⁺ vector primers. DNA from each clone was spotted in duplicate onto a single microarray slide. Appropriate positive and negative controls were also incorporated onto the plate.

### Probe Labelling

Human umbilical vein endothelial cells harvested at the specified time points (0, 0.5, 3, 6, and 24 hr) were used for the isolation of total RNA using the Trizol reagent (Gibco BRL) according to manufacturers conditions. From each time point, 0.5 ug of total RNA was used as a template for the amplification of antisense RNA (aRNA) using the Ambion MessageAmp™ aRNA Kit. Briefly, total RNA was reversed transcribed with a T7 oligo(dT) primer in order to synthesize cDNA containing a T7 promoter sequence extending from the poly(A) tails of messages generated by reverse transcription. The cDNA was converted to a double-stranded DNA template and used for in vitro transcription of aRNA, incorporating 5-(3-aminoallyl)-UTP so as to allow coupling of fluorescent CyDyes. A typical amplification reaction would yield approximately 10 ug of mRNA (>400× amplification, assuming the initial total RNA contained <5% mRNA).

### Microarray Hybridization

After coupling of CyDyes, the synthesized aRNA was used as a probe (3.0-3.5 ug) for hybridization to a microarray slide. The hybrizations performed were as follows:

1. 0 vs 0.5 h (6 slides, 3 Dye swaps)

2. 0 vs 3 h (4 slides, 2 Dye swaps)

3. 0 vs 6 h (4 slides, 2 Dye swaps)

4. 0 vs 24 h (4 slides, 2 Dye swaps)

Multiple slides were hybridized for each time point in order to verify the result from any one hybridization. Slides were hybridized in chambers for 16 hours, washed, and then scanned using the GenePix 2000 scanner. Those clones that were shown to be highly up-regulated were chosen for further analysis.

In summary, SSH was used in combination with microarray analyses to identify genes that are up-regulated and may be involved in biological processes underlying endothelial cell activation and blood vessel formation. This approach is novel in that it involves nucleotide hybridization steps that aim to reduce gene detection redundancy and enhance the chances of detecting genes that are of low overall representation in the endothelial cell transcriptome. The nucleotide-based sequential time-points aims to detect the timepoint at which the up-regulation of a particular gene takes place in a way that reduces redundancy of detection. For example, a gene that is up-regulated at 3 hrs, and its expression remains up-regulated in subsequent time-points, will only be detected in the 0.5-3 hr subtraction step. In contrast, if subtractions were done with the 0 hr timepoint for all subsequent timepoints then this example gene would be detected at all subtraction steps following the 3 hrs timepoint subtraction. This would introduce redundancy that could result in masking the possible detection of other genes of lower representation in the endothelial cell mRNA expression pool. The subsequent use of microarray analysis is based on the comparison subtraction hybridization in the SSH step involving each timepoint with the 0 hrs timepoint. This enables the expression profiling of each gene across all timepoints in relation to 0 hrs, irrespective of the timepoint at which it is up-regulated.

### Example 5

### Clone Selection

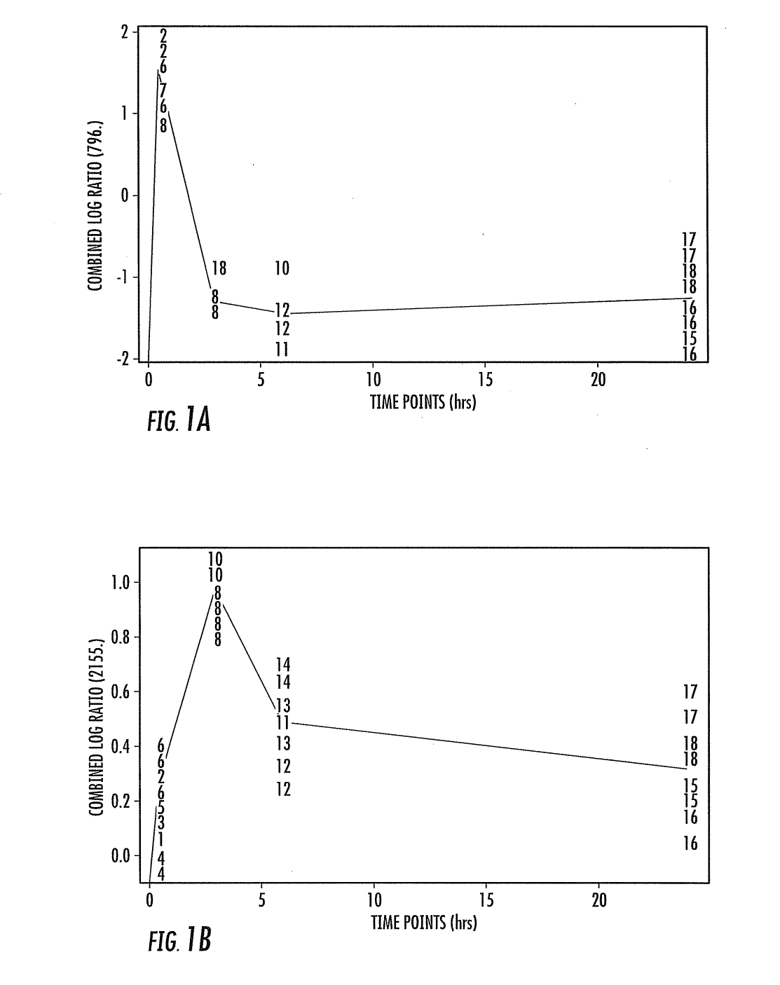

From analysis of the microarray hybridizations, a total of 1,963 clones were identified to be up-regulated in their expression at specified time points during the in vitro model of angiogenesis. FIG. 1 provides an example of the expression profiles observed during defined time points in the in vitro model for a selection of clones. Each of the 1,963 clones were sequenced and subsequent in silico database analysis was used to remove clones containing vector sequences only and clones for which poor sequence was obtained. Following this, redundancy screens were used to group clones according to individual genes that they represented. This left a total of 523 genes that were found to be up-regulated in their expression during the process of angiogenesis.

Tables 1, 2 and 3 provide information on the up-regulated clones that were sequenced. Table 1 includes those clones which represent previously uncharacterised or novel genes, while Table 2 includes clones that correspond to previously identified genes which have not before been associated with angiogenesis. Also identified were a number of genes that have previously been shown to be involved in the process of angiogenesis (Table 3). The identification of these clones provides a validation or proof of principle of the effectiveness of the angiogenic gene identification strategy employed and suggests that the clones listed in Tables 1 and 2 are additional angiogenic gene candidates.

### Example 6

### Analysis of the Angiogenic Genes

Further evidence for the involvement of the genes in Tables 1 and 2 in angiogenesis can be obtained through the functional analysis of each gene, for example by examining the effect that knock-down of their expression has on endothelial cell (EC) function and capillary tube formation.

A number of knock-down technologies and assays may be used. For example full-length coding sequences of the genes can be cloned into suitable expression vectors such as retroviruses or adenoviruses in both sense and anti-sense orientations and used for infection into ECs. Retrovirus infection gives long-term EC lines expressing the gene of interest whereas adenovirus infection gives transient gene expression. Infected cells can then be subjected to a number of EC assays including proliferation and capillary tube formation to confirm the role of each gene in angiogenesis.

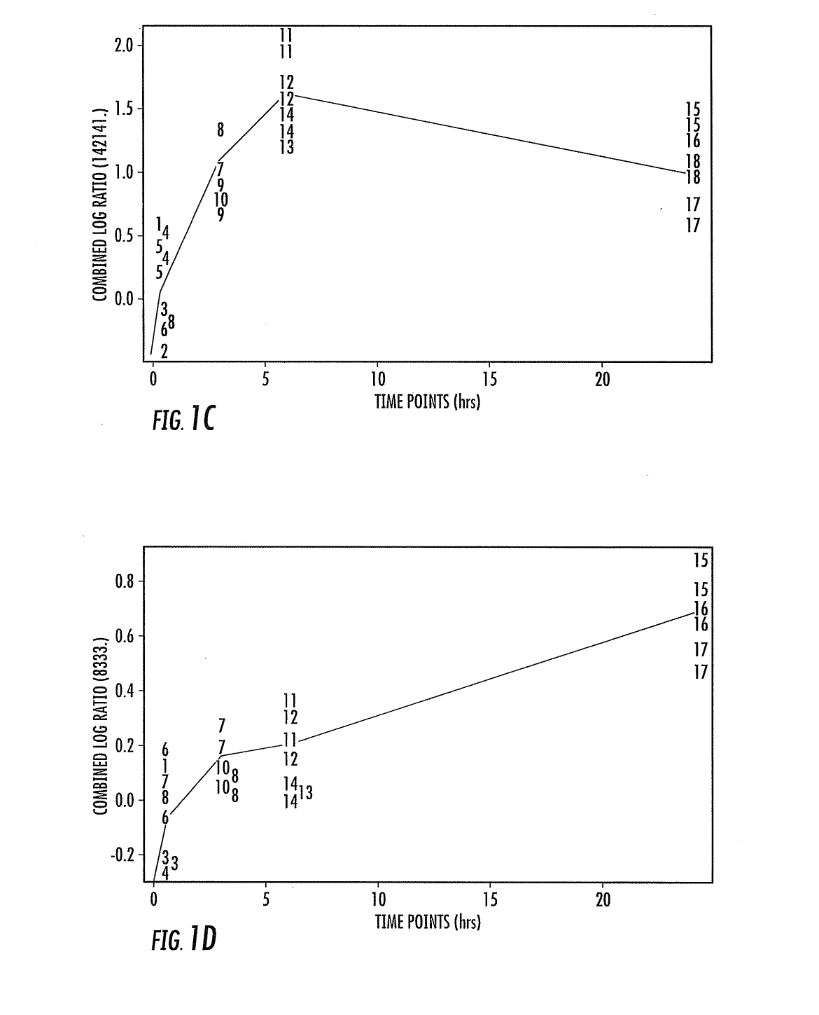

In this study RNA interference (RNAi) gene knock-down technology was used for the analysis of gene function (see detailed description below). In this technique, short gene-specific RNA oligonucleotides are delivered to ECs in culture mediated by retroviral infection. These oligonucleotides bind to the gene transcript under study and induce its degradation resulting in silencing or reduction of gene expression. The consequences of this alteration to gene expression can be subsequently studied using assays that examine the ability of ECs to proliferate, migrate and form capillaries in vitro. The RNAi procedure adopted in this study is described below in detail and documents the analysis of two of the identified up-regulated angiogenesis genes. One of these genes is BNO782 shown in Table 1, a novel gene whose expression peaks at the 6 hour time point of the in vitro angiogenesis model (FIG. 2A), while the other gene is BNO481 (KPNA4) as shown in Table 2, which is a previously identified gene that has not before been shown to have a role in angiogenesis. The expression of BNO481 also peaks at the 6 hour time point of the in vitro angiogenesis model (FIG. 2B).

### RNAi Oligonucleotide Design

Short interfering RNA (siRNA) oligonucleotides for RNAi-mediated knock-down of BNO782 and BNO481 were identified through application of in-house computer software. This software incorporates a series of parameters for selecting appropriate siRNA oligonucleotides. These parameters ensure that the siRNA sequence starts after an AA dinucleotide, the siRNA is in the open reading frame of the gene and 100 bp downstream the ATG start codon, the GC content of the siRNA is between 35% and 60%, and the siRNA does not have stretches of more than three T, A, C or G nucleotides. siRNA sequences that harbour low complexity regions were not used. In addition, BLAST analysis was used to select against probes that cross-hybridize with a number of genes (Blastn_refseq at “expect 500” and “word size 7” and alignment scores accepted at 19>score>15 where: alignment score=length_match−(gap+mismatch). siRNAs were synthesised in hair-pin format for cloning into retroviral vectors. For each gene, three siRNA oligonucleotides were selected with each one being examined individually for their effects on gene-knock-down and EC function.

### Retroviral Infection of HUVE Cells

Each siRNA oligonucleotide was cloned into a retroviral vector for the delivery of the oligonucleotide to human umbilical vein endothelial cells (HUVECs). The siRNA vector was constructed through a modification of pMSCVpuro (BD Biosciences). Briefly, the 3′LTR of pMSCVpuro was inactivated by removal of the XbaI/NheI fragment. A H1-RNA Polymerase III promoter cassette was then inserted into the MCS of the vector. Annealed siRNA primers were ligated into the modified vector (pMSCVpuro(H1)) digested with BglII and HindIII restriction enzymes.

For virus production prior to infection of HUVECs, 293T cells were plated at a density of 1×10⁶ cells per well of a 6 well plate 18-24 hours before transfection in RPMI media (Invitrogen) supplemented with 10% FCS (Invitrogen) and 1.0 M Hepes (Invitrogen) without antibiotics. Cells were co-transfected with 2 μg retroviral DNA and 1.5 μg pVPack-VSV-G (Stratagene), 1.5 μg pVPack-GP (Stratagene) using Lipofectamine 2000 reagent (Invitrogen). Transfected cells were incubated overnight in 5% CO₂ at 37° C. The following day, media containing the DNA/LF2000 complexes was removed and replaced with RPMI supplemented with 10% FCS, 1.0 M Hepes and 1% PSG (Invitrogen). Virus containing supernatants were collected 48-72 hours post transfection and filtered using a 0.45 μM filter. Virus was aliquoted and stored at −80° C.

For the retroviral infection of HUVECs (Clonetics), cells were plated 24 hours before infection in EGM-2 media (Clonetics) at a density of 1.3×10⁵ cells per well of a 6 well plate. The following day, 500 μl of virus supernatant was combined with 500 μl of EGM-2 complete media. Polybrene (Sigma) was added to a final concentration of 8.0 μg/ml. Media was aspirated from the cells and replaced with the viral mix. Cells were incubated with the viral mix in 5% CO₂ at 37° C. After 3 hours incubation, an additional 1.0 ml of EGM-2 media was added and cells were incubated for a further 24 hours. After this time HUVE cells were split 1:2 and replated into a 6 well plate. Cells were incubated for 24 hours following splitting to allow them to recover and adhere. To select for infected cells, medium was replaced with EGM-2 complete medium containing puromycin (Sigma) at a 0.4 μg/ml final concentration. Cells were incubated until uninfected cells treated with puromycin had died and infected resistant cells had grown to confluence. Media containing puromycin was replaced every 48 hours to replenish puromycin and remove cell debris. Once resistant cells were grown to confluence (approximately 4-5 days after starting selection), cells were washed in PBS, trypsinised and their properties analysed using the Matrigel capillary tube formation assay.

### Capillary Tube Formation Assay

96 well tissue culture plates were coated with 50 μl of cold Matrigel (BD Biosciences) at 4° C. in a two layer process. Matrigel was allowed to polymerize at 37° C. for a minimum of 30 minutes before being used. Trypsinised cells were collected in 500 μl of EGM-2 media then centrifuged at 400 rcf for 3 minutes to pellet cells. This allows for the removal of trypsin that may interfere with the assay. Cell pellets were resuspended in 500 μl EGM-2 media then counted using a heamocytometer. Cells were diluted to 2.5×10⁵ cells/ml in EGM-2 media. 100 μl of the diluted cell suspension was added to duplicate Matrigel coated wells. The final cell density was 25,000 cells/well. Plates were incubated for 22 hours in a humidified incubator at 37° C. with 5% CO₂. Images were obtained using an Olympus BX-51 microscope with a 4× objective and Optronics MagnaFire software. Remaining cells were pelleted at 400 rcf for 3 minutes, then media was removed and pellets stored at −80° C. for extraction of RNA for real-time RT-PCR analysis (see below). For all assays performed, a vector control was included. This consisted of HUVECs undergoing the infection and selection process with virus made for the vector containing no siRNA insert. This allows for comparison of capillary tube formation ability between a control (vector) and the individual siRNA under analysis.

### Real-Time RT-PCR Analysis

To determine the level of gene knock-down (mediated by the siRNAs) occurring in the HUVECs, real-time RT-PCR was employed. This involved isolation of RNA from infected cells using the RNeasy Mini or Midi kits (Qiagen) as per manufacturer's instructions (including the on-column DNase treatment). Total RNA was visualised on a 1.2% TBE agarose gel containing ethidium bromide to check for quality and purity. Total RNA concentration was determined by A₂₆₀ on a spectrophotometer.

For the synthesis of cDNA, total RNA (at least 1 ug and preferably at a concentration >1.0 ug/ul) was reverse transcribed using M-MLV (Promega) as per manufacturer's directions. Briefly, the RNA sample to be analysed was made up to 13 ul with water and 1.0 ul of oligo-dT primer (500 ng/ul) was added. After incubating at 70° C. for 5 minutes, the tubes were placed on ice for 5 minutes and 11 ul of a pre-made master mix containing 5.0 ul M-MLV RT 5× Reaction Buffer, 1.25 ul 10 mM dNTP mix, 1.0 ul of M-MLV RT (H″ point mutant) enzyme, and 3.75 ul water was added. This mix was incubated at 40° C. for one hour, and the reaction terminated by incubating at 70° C. for 15 minutes.

Real-Time PCRs were run on the RotorGene™ 2000 system (Corbett Research). Reactions used AmpliTaq Gold enzyme (Applied Biosystems) and followed the manufacturers instructions. Real-Time PCR reactions were typically performed in a volume of 25 ul and consisted of 1× AmpliTaq Gold Buffer, 200 nM dNTP mix, 2.0 mM MgCl₂ (may vary for primer combination used), 0.3 uM of each primer, 1×SYBR Green mix (Cambrex BioScience Rockland Inc), 1.2 ul of AmpliTaq Gold Enzyme, and 10 ul of a 1 in 5 dilution of the cDNA template.

Cycling conditions were typically performed at 94° C. for 12 minutes, followed by 35 cycles of 94° C. for 15 seconds, 60° C. for 15 seconds, and 72° C. for 20 seconds. The annealing temperature of the primers may vary depending on the properties of the primers used.

The PCR cycling was followed by the generation of a melt curve using the RotorGene™ 2000 software where the amount of annealed product was determined by holding at each degree between 50° C. and 99° C. and measuring the absorbance. All products were run on a 1.2% agarose gel containing ethidium bromide to check specificity in addition to observing the melt curve.

The level of knock-down of a particular gene was then measured by a comparison of its expression level in HUVECs infected with the relevant siRNA under investigation as opposed to HUVECs infected with the retroviral vector alone.

### In Vitro Regulation of HUVEC Function—BNO782 and BNO481

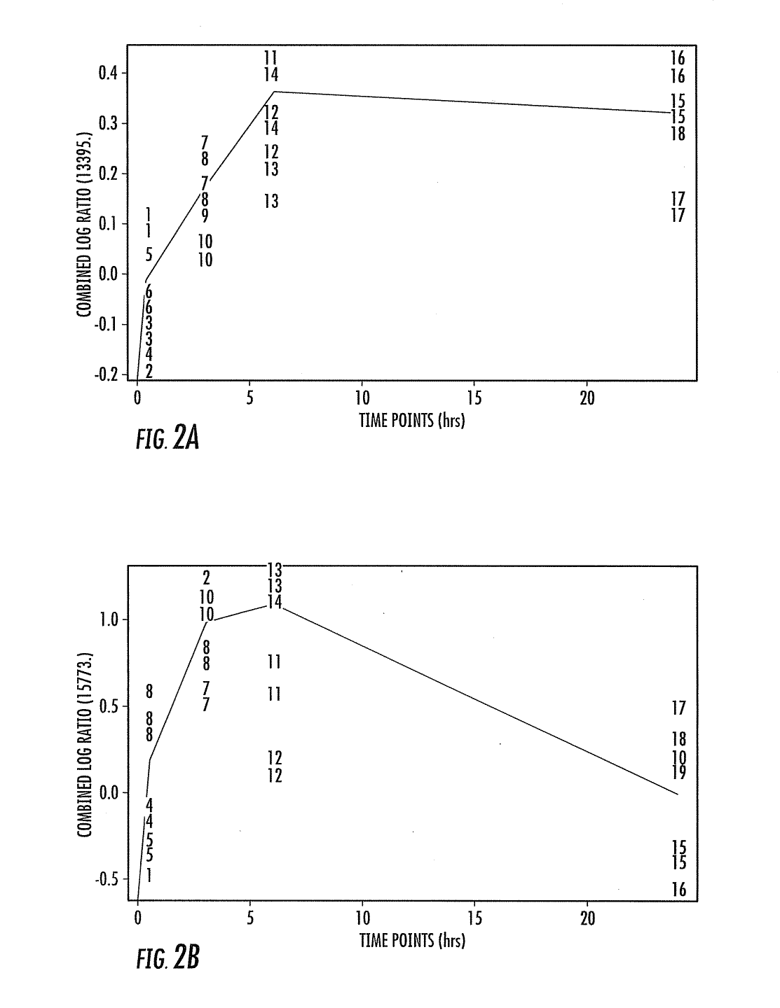

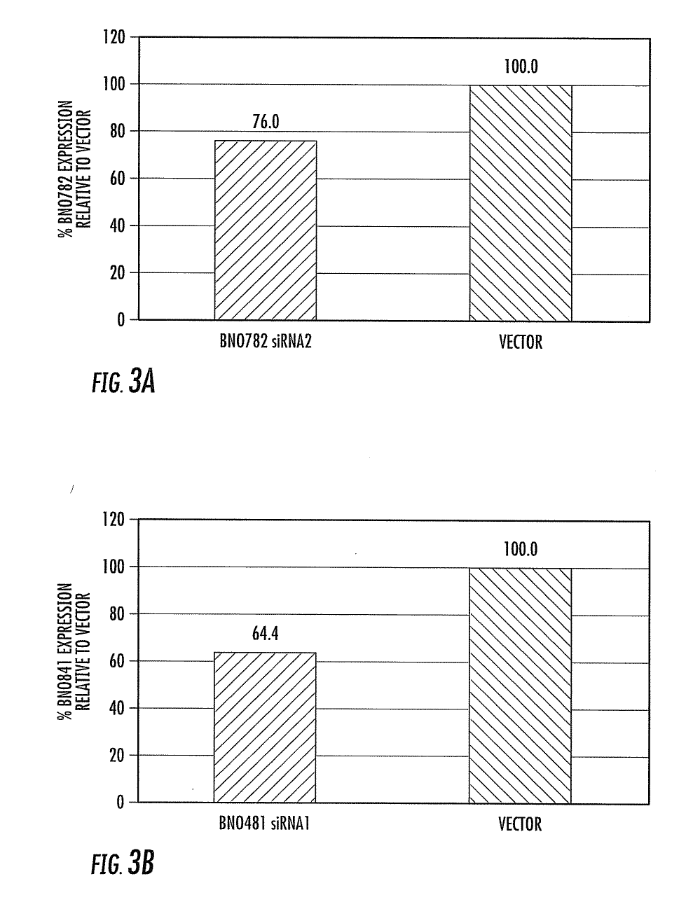

The siRNA oligonucleotides designed to knock-down BNO782 and BNO481 expression are represented by SEQ ID Numbers: 45-47 and SEQ ID Numbers: 48-50 respectively. Real-time RT-PCR analysis of HUVECs retrovirally infected with these siRNAs revealed that each siRNA was able to knock-down the expression of BNO782 or BNO481 to varying degrees. The level of BNO782 expression knock-down mediated by BNO782 siRNA2 (SEQ ID NO: 46) was 24% (FIG. 3A), while expression of BNO481 was reduced by 36% (FIG. 3B) using BNO481 siRNA1 (SEQ ID NO: 48). Both of these siRNAs were subsequently used separately in Matrigel assays to examine the effects that this level of knock-down for each gene had on the ability of HUVECs to participate in capillary tube formation. As can be seen in FIG. 4, reducing BNO782 or BNO481 mRNA levels inhibits HUVEC tube formation. Vector infected cells formed extensive networks of tube structures (FIGS. 4A and 4C) while cells infected with BNO782 siRNA2 or BNO481 siRNA1 exhibited tube structure networks of significantly reduced complexity with a high number of incomplete tube extensions (FIGS. 4B and 4D). This result confirms a role for both BNO782 and BNO481 in the process of angiogenesis.

### Protein Interaction Studies

The ability of any one of the angiogenic proteins of the invention, including BNO782 and BNO481, to bind known and unknown proteins can be examined. Procedures such as the yeast two-hybrid system are used to discover and identify any functional partners. The principle behind the yeast two-hybrid procedure is that many eukaryotic transcriptional activators, including those in yeast, consist of two discrete modular domains. The first is a DNA-binding domain that binds to a specific promoter sequence and the second is an activation domain that directs the RNA polymerase II complex to transcribe the gene downstream of the DNA binding site. Both domains are required for transcriptional activation as neither domain can activate transcription on its own. In the yeast two-hybrid procedure, the gene of interest or parts thereof (BAIT), is cloned in such a way that it is expressed as a fusion to a peptide that has a DNA binding domain. A second gene, or number of genes, such as those from a cDNA library (TARGET), is cloned so that it is expressed as a fusion to an activation domain. Interaction of the protein of interest with its binding partner brings the DNA-binding peptide together with the activation domain and initiates transcription of the reporter genes. The first reporter gene will select for yeast cells that contain interacting proteins (this reporter is usually a nutritional gene required for growth on selective media). The second reporter is used for confirmation and while being expressed in response to interacting proteins it is usually not required for growth.

The nature of the interacting genes and proteins can also be studied such that these partners can also be targets for drug discovery.

### Structural Studies

Recombinant angiogenic proteins of the invention can be produced in bacterial, yeast, insect and/or mammalian cells and used in crystallographical and NMR studies. Together with molecular modeling of the protein, structure-driven drug design can be facilitated.

### Example 7

### BNO802

In order to select for such genes an assay that involves the formation of blood capillary counterparts in an extracellular matrix medium derived from tumour cells was used. In this assay endothelial cells were incubated on an extracellular matrix derived from mouse sarcoma cells (Matrigel). Endothelial cells cultured on Matrigel matrices undergo sequential morphological changes culminating in the formation of tube structures that are thought to represent a good in vitro model of blood capillaries. This in vitro behaviour constitutes a phenotypic manifestation of a number of temporally and well-orchestrated cellular events that involve endothelial cell activation, migration and cellular remodelling. Endothelial cell proliferation is a key process underlying the early stages of blood capillary formation. Any compounds interfering with this process are likely to inhibit angiogenesis. The differentiation of endothelial cells into networks of tubes in vitro is preceded by a stop in proliferation. Laminin is one of the components of the extracellular matrix in the natural mileu of the endothelial cell as well as in the Matrigel matrix responsible for imparting anti-proliferative signals and enhancing differentiation into tube structures. Consequently, the tube formation assay does not encompass the proliferative events that take place during parts of the angiogenic process. Thus the proliferation assay was used to complement the Matrigel assay for the validation of potential angiogenesis targets.

Using this strategy, a gene is considered to be involved in angiogenesis, and potentially a good drug target, if the RNAi-mediated silencing of the gene resulted in the inhibition of endothelial cell capillary formation on Matrigel and/or is the inhibition of endothelial cell proliferation in culture.

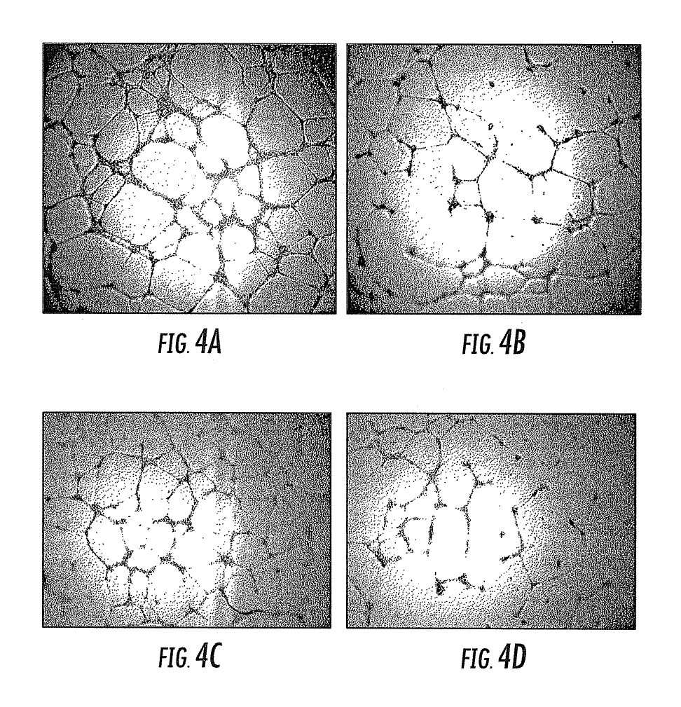

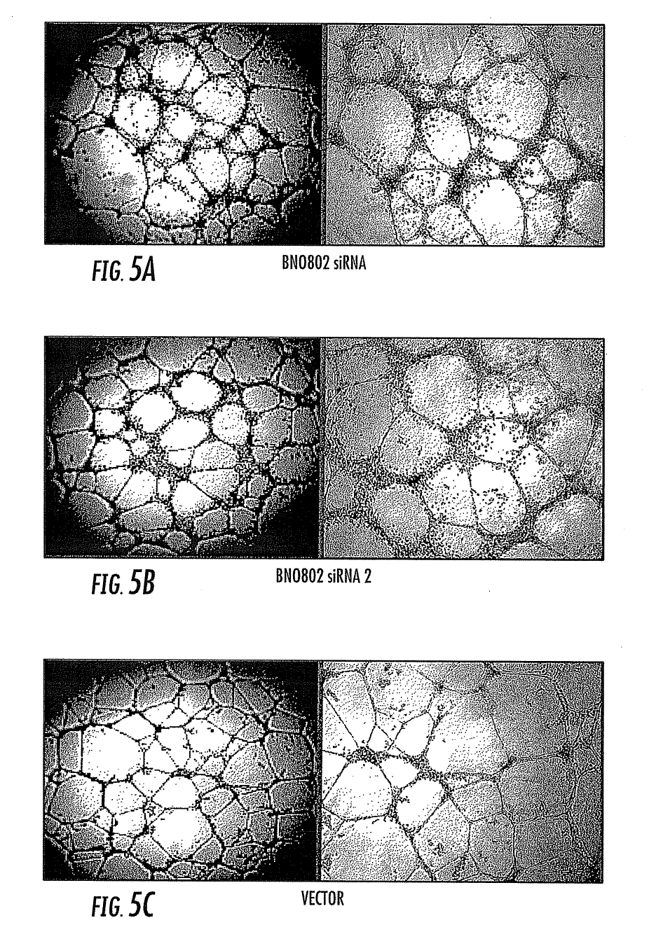

FIG. 5 gives an evaluation of the consequences of siRNA-mediated knockdown of BNO802 on the formation of capillary tubes by endothelial cells on Matrigel. The pictures demonstrate a significant reduction in capillary formation. The observation of FIG. 5 is quantified in the graph of FIG. 6, which gives an evaluation of the consequences of siRNA-mediated knockdown of BNO802 on the ability of endothelial cells to proliferate. Again, a significant knockdown is demonstrated in siRNA.

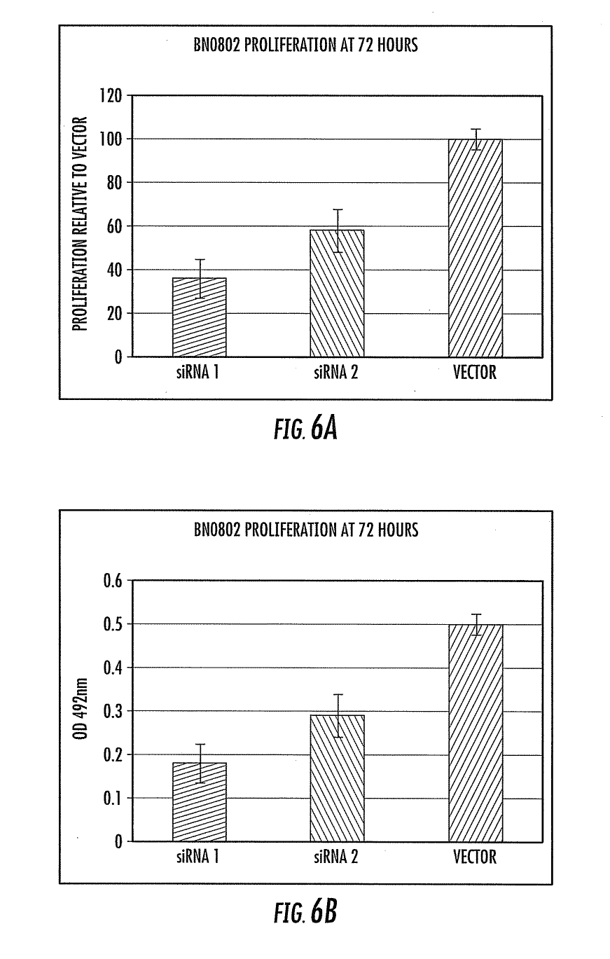

FIG. 7 is a realTime-RTPCR analysis evaluating the degree of BNO802 gene knockdown achieved with RNAi. Total RNA was extracted from cells and reverse transcribed into cDNA followed by RealTime PCR amplification using gene specific primers. Expression levels were normalised to the house-keeping gene POLR2K and expressed as a percent of the vector control (n=3). The analysis is as performed in example 5 and the level of knockdown of a particular gene is measured by comparison of its expression level in cells infected with the relevant siRNA as opposed to those infected with retroviral vector alone. FIG. 7 demonstrates a reduction in expression to a level of less than 20 percent.

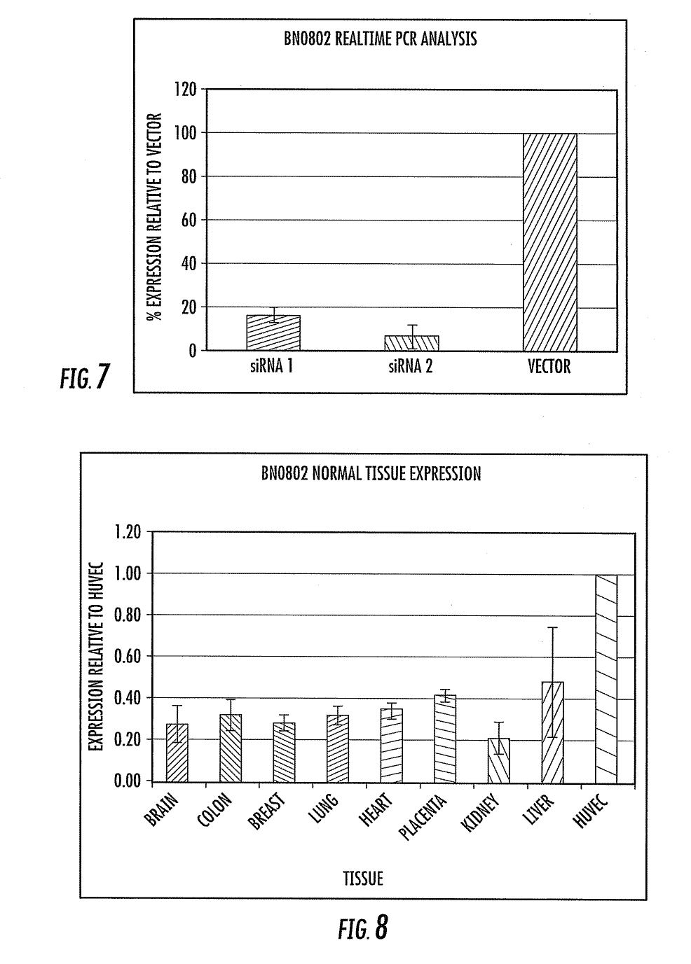

FIG. 8 provides an evaluation of BNO802 gene expression in normal human tissues using RealTime RTPCR analysis. Human RNA samples (Ambion) were reverse transcribed into cDNA followed by RealTime PCR using gene specific primers. Gene expression data was normalised to the expression of the house-keeping gene POLR2K. The level of gene expression in each tissue was expressed relative to the gene expression found in a homogeneous endothelial cell population (HUVEC) (n=4).

### REFERENCES

References cited herein are listed on the following pages, and are incorporated herein by this reference.
Altschul, S F. et al. (1997). Nucleic Acids Res. 25: 3389-3402.
Breaker, R R. and Joyce, G F. (1995). Chem. Biol. 2: 655-600.
Cole, S R et al. (1984). Mol. Cell Biol. 62: 109-120.
Cote, R J. et al. (1983). Proc. Natl. Acad. Sci. USA 80: 2026-2030.
Culver, K. (1996). Gene Therapy: A Primer for Physicians. Second Edition. (Mary Ann Liebert).
Folkman, J. and Haudenschild, C. (1980). Nature (Lond.) 288: 551-556.
Friedman, T. (1991). In Therapy for Genetic Diseases. (T Friedman (Ed) Oxford University Press. pp 105-121.
Gamble, J R. et al. (1993). J. Cell Biol. 121: 931-943.
Gamble, J R. et al. (1999). Endothelium 7: 23-34.
Gecz, J. et al. (1997). Genomics 44: 201-213.
Goldman, C K. et al. (1997). Nature Biotechnology 15: 462-466.
Green, L L. et al. (1994). Nature Genet. 7: 13-21.
Grishok, A. et al. (2001). Science 287: 2494-2497.
Haseloff, J. and Gerlach, W L. (1988). Nature 334: 585-591.
Heller, R A. et al. (1997). Proc. Natl. Acad. Sci. USA 94: 2150-2155.
Huse, W D. et al. (1989). Science 246: 1275-1281.
Kohler, G. and Milstein, C. (1975). Nature 256: 495-497.
Kozbor, D. et al. (1985). J. Immunol. Methods 81:31-42.
Lonberg, N. et al. (1994). Nature 368: 856-859.
Meyer, G T. et al. (1997). The Anatomical Record 249: 327-340.
Orlandi, R. et al. (1989). Proc. Natl. Acad. Sci. USA 86: 3833-3837.
Rickert, R C. et al. (1997). Nucleic Acids Res. 25: 1317-1318.
Sambrook, J. et al. (1989). Molecular cloning: a laboratory manual. Second Edition. (Cold Spring Harbour Laboratory Press, New York).
Scharf, D. et al. (1994). Results Probl. Cell Differ. 20: 125-162.
Schena, M. et al. (1996). Proc. Natl. Acad. Sci. USA 93: 10614-10619.
Schwenk, F. et al. (1995). Nucleic Acids Res. 23: 5080-5081.
Sharp, P A. and Zamore, P D. (2000). Science 287: 2431-2432.
Taylor, L D. et al. (1994). Int. Immunol. 6: 579-591.
Winter, G. et al. (1991). Nature 349: 293-299.

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of modulating angiogenesis comprising modulating the expression or activity of a BNO802 polypeptide in a cell, wherein the BNO802 polypeptide is encoded by a BNO802 nucleic acid molecule set forth in Table 1.

2. The method of claim 1, wherein the expression or activity of the BNO802 polypeptide is modulated by introducing into the cell an antisense to the BNO802 nucleic acid molecule.

3. The method of claim 1, wherein the expression or activity of the BNO802 polypeptide is modulated by introducing into the cell a nucleic acid which is an siRNA.

4. The method of claim 1, wherein the expression or activity of the BNO802 polypeptide is modulated by an antibody capable of binding the BNO802 polypeptide.

5. The method of claim 4, wherein the antibody is a fully human antibody.

6. The method of claim 4, wherein the antibody is selected from the group consisting of a monoclonal antibody, a humanised antibody, a chimaeric antibody or an antibody fragment including a Fab fragment, (Fab′)₂ fragment, Fv fragment, single chain antibodies and single domain antibodies.

7. A method for the treatment of an angiogenesis-related disorder, comprising modulating the expression or activity of a BNO802 polypeptide encoded by a BNO802 nucleic acid molecule set forth in Table 1.

8. The method of claim 7, wherein the expression or activity of the BNO802 polypeptide is modulated by introducing into the cell an antisense to the BNO802 nucleic acid molecule.

9. The method of claim 7, wherein the expression or activity of the BNO802 polypeptide is modulated by introducing into the cell a nucleic acid which is an siRNA.

10. The method of claim 7, wherein the expression or activity of the BNO802 polypeptide is modulated by an antibody capable of binding the BNO802 polypeptide.

11. The method of claim 10, wherein the antibody is a fully human antibody.

12. The method of claim 10, wherein the antibody is selected from the group consisting of a monoclonal antibody, a humanised antibody, a chimaeric antibody or an antibody fragment including a Fab fragment, (Fab′)₂ fragment, Fv fragment, single chain antibodies and single domain antibodies.

13. The method of claim 7, wherein the disorder is selected from the group consisting of cancer, rheumatoid arthritis, diabetic retinopathy, psoriasis, and cardiovascular diseases such as atherosclerosis, ischaemic limb disease or coronary artery disease.

14. A method of screening for a candidate pharmaceutical compound for the treatment of an angiogenesis-related disorder, comprising the steps of:
(1) providing a BNO802 polypeptide set forth in Table 1;
(2) adding a candidate pharmaceutical compound to said BNO802 polypeptide; and
(3) determining the binding of said candidate compound to said BNO802 polypeptide;
wherein a compound that binds to the polypeptide is a candidate for the treatment of an angiogenesis-related disorder.

15. A method of screening for a candidate pharmaceutical compound useful in the treatment of an angiogenesis-related disorder, comprising the steps of:
(1) providing a cell transformed with an expression vector comprising a BNO802 nucleic acid molecule set forth in Table 1;
(2) adding a candidate pharmaceutical compound to said cell; and
(3) determining the effect of said candidate pharmaceutical compound on the expression or activity of the polypeptide encoded by the BNO802 nucleic acid molecule that is part of the expression vector in said cell;
wherein a compound that alters the expression or activity of the polypeptide encoded by the BNO802 nucleic acid molecule that is part of the expression vector in said cell is a candidate for the treatment of an angiogenesis-related disorder.
